(19) United States
(12) Patent Application Publication
(10) Pub. No.: US 2026/0026988 A1
(43) Pub. Date: Jan. 29, 2026

(54) BADGE AND PATIENT SUPPORT APPARATUS COMMUNICATION SYSTEM

(71) Applicant: Stryker Corporation, Kalamazoo, MI (US)

(72) Inventors: Jerald A. Trepanier, Augusta, MI (US); Kirby M. Neihouser, Portage, MI (US); Krishna Sandeep Bhimavarapu, Kalamazoo, MI (US); Madhu Sandeep Thota, Portage, MI (US); Celso Henrique Farnese Pires Pereira, Portage, MI (US); Madhu Thomas, London, Ontario (CA); Christopher P. Alvarez, Kalamazoo, MI (US); Anish Paul, Portage, MI (US); Tyler Joseph Ethen, Portage, MI (US); Nicholas S. Brajak, Portage, MI (US)

(21) Appl. No.: 18/724,560

(22) PCT Filed: Jun. 28, 2023
(86) PCT No.: PCT/US2023/026440

§ 371 Date: Jun. 26, 2024

Related U.S. Application Data
(60) Provisional application No. 63/356,061, filed on Jun. 28, 2022.
(60) Provisional application No. 63/356,065, filed on Jun. 28, 2022.
(60) Provisional application No. 63/356,238, filed on Jun. 28, 2022.
(60) Provisional application No. 63/357,363, filed on Jun. 30, 2022.

(51) Int. Cl. A61G 7/05; A61B 5/00; A61B 5/11; G16H 40/67; H04B 1/7163; H04L 67/12; H04W 4/029; H04W 4/38

(52) U.S. Cl. CPC A61G 7/0524; A61B 5/1115; A61B 5/1117; A61B 5/7275; A61B 5/746; G16H 40/67; H04B 1/7163; H04L 67/12; H04W 4/029; H04W 4/38; A61G 2205/60

(57) ABSTRACT

A patient support apparatus includes a patient support surface, a plurality of ultra-wideband transceivers, and a controller. The controller is adapted to use the ultra-wideband transceivers to automatically determine a position of the badge relative to the patient support apparatus, to receive a badge identifier from the badge, to determine if the badge is positioned inside or outside of a volume of space; and, if the badge is positioned inside the volume of space, to perform one or more of the following: stop an alert that would otherwise be issued; determine a distance between the badge and a tag worn by the patient; send the badge identifier and status data to an offboard device that includes a dashboard screen; determine if a correct type of healthcare worker is present at the patient support apparatus; send the badge identifier to a server; and/or take other actions.

### BACKGROUND

This application claims priority to the following U.S. provisional patent applications: Ser. No. 63/356,065 filed Jun. 28, 2022, by inventors Jerald Trepanier et al. and entitled BADGE AND PATIENT SUPPORT APPARATUS COMMUNICATION SYSTEM; Ser. No. 63/356,061 filed Jun. 28, 2022, by inventors Krishna Sandeep Bhimavarapu et al. and entitled BADGE AND PATIENT SUPPORT APPARATUS COMMUNICATION SYSTEM; Ser. No. 63/356,238 filed Jun. 28, 2022, by inventors Madhu Sandeep Thota et al. and entitled BADGE AND PATIENT SUPPORT APPARATUS COMMUNICATION SYSTEM; and Ser. No. 63/357,363 filed Jun. 30, 2022, by inventors Madhu Sandeep Thota et al. and entitled BADGE AND PATIENT SUPPORT APPARATUS COMMUNICATION SYSTEM, the complete disclosures of all of which are incorporated herein by reference.

### BACKGROUND

The present disclosure relates to patient support apparatuses, such as beds, cots, stretchers, recliners, or the like. More specifically, the present disclosure relates to patient support apparatuses that communicate with one or more badges, tags, and/or other devices that are separate from the patient support apparatus itself.

### SUMMARY

According to the various aspects described herein, the present disclosure is directed to a patient support apparatus system that monitors the positions of badges worn by one or more different types of users, such as, but not limited to, caregivers, service technicians, cleaners, transportation assistants, etc. The patient support apparatus system may also, or alternatively, monitor the positions of equipment tags attached to one or more pieces of equipment, the positions of fixed locators mounted at different locations within a healthcare facility, and/or the positions of one or more UWB-equipped smart phones. The system may include one or more servers and one more patient support apparatuses adapted to communicate with the server(s), the badges, the equipment tags, the fixed locators, and/or the smart phones. The patient support apparatuses may use the positions of the badges, tags, fixed locators, and/or smart phones to perform one or more functions. These functions include, but are not limited to, automatically enabling and/or disabling alerts issued from patient support apparatuses; automatically checking to see that the correct healthcare worker, or set of healthcare workers, responds to important event, such as, but not limited to, cardiac arrests of the patient; automatically sharing patient support apparatus status data and healthcare worker location information with a device configured to display such information on a consolidated dashboard screen; automatically monitoring the distance between caregivers and their patients, including after the patients exit from their respective patient support apparatuses; automatically determining types of caregivers associated with badges; automatically determining which caregiver of a certain type is positioned closest to a patient support apparatus needing attention; automatically determining whether the patient support apparatus is being moved into or out of an area of the healthcare facility; automatically logging a time sequence of events, equipment, and personnel when a state of the patient support apparatus is changed; automatically preventing movement of one or more components of the patient support apparatus if a person or object is within a movement path of the one or more components; and/or automatically taking other actions. These and other aspects of the present disclosure will be apparent to a person of ordinary skill light of the following written description and accompanying drawings.

According to a first aspect of the present disclosure, a patient support apparatus is provided that includes a support surface adapted to support a patient, a plurality of ultra-wideband transceivers, a sensor adapted to detect an undesired state of the patient support apparatus, a network transceiver adapted to communicate with a healthcare facility computer network, and a controller. The controller is adapted to use radio frequency (RF) communication between the plurality of ultra-wideband and a badge to determine a position of the badge relative to the patient support apparatus. The controller is further adapted to receive a badge identifier from the badge; to determine if the badge is positioned inside or outside of a volume of space; to issue an alert if the badge is positioned outside of the volume of space while the undesired state exists; and to stop the alert if the badge is positioned inside the volume of space while the undesired state exists.

According to other aspects of the present disclosure, the controller is configured to stop the alert for as long as the badge remains inside the volume of space and the undesired state continues to exist.

In some aspects, the sensor is adapted to detect an exit of the patient from the patient support apparatus, and the undesired state is the patient exiting from the patient support apparatus.

The patient support apparatus, in some aspects, further includes a plurality of siderails movable between raised and lowered positions, and the sensor is adapted to detect a position of at least one of the siderails. The undesired state may involve at least one of the siderails being moved to its lowered position.

The patient support apparatus, in some aspects, further includes a sound emitting device, and the controller is further adapted to cause the sound emitting device to emit an alert sound when the controller issues the alert, and to cause the sound emitting device to not emit the alert sound when the controller stops the alert.

The controller, in some aspects, is adapted to issue the alert by sending an alert message to a server hosted on the healthcare facility network, and to stop the alert by sending an alert cancellation message to the server hosted on the healthcare facility network.

In some aspects, the patient support apparatus further includes comprising a headwall interface adapted to communicate with a communication outlet integrated into a wall of the healthcare facility. The communication outlet includes a plurality of pins and the controller is adapted to issue the alert by setting an electrical state of at least one pin to a first state, and to stop the alert by setting the electrical state of the at least one pin to a second state.

The controller, in some aspects, is further adapted to issue the alert by sending an alert message to the badge, and to stop the alert by sending a cancellation message to the badge.

In some aspects, the controller is further adapted to use RF communication between the plurality of ultra-wideband transceivers and a fixed locator to determine a position of the patient support apparatus relative to the fixed locator.

In some aspects, the controller is further adapted to receive a locator identifier from the fixed locator, to determine if the patient support apparatus is positioned within a threshold distance of the fixed locator, and to forward the locator identifier to the healthcare facility computer network via the network transceiver if the patient support apparatus is positioned within the threshold distance.

The patient support apparatus, in some aspects, further includes a microphone positioned onboard the patient support apparatus. The microphone is adapted to convert sounds of the patient's voice to audio signals, and the controller is further adapted to transmit the audio signals to the fixed locator if the fixed locator is positioned within the threshold distance, and to not transmit the audio signals to the fixed locator if the fixed locator is positioned outside of the threshold distance.

In some aspects, the controller is further adapted to send a first message to a server via the network transceiver if the badge is positioned inside the volume of space, and to send a second message to the server via the network transceiver if the badge is positioned outside of the volume of space, wherein both the first and second messages include the badge identifier

The controller, in some aspects, is adapted to send status data regarding the patient support apparatus to the server via the network transceiver.

The status data, in some aspects, includes one or more of the following: a status of a siderail on the patient support apparatus; a status of a brake on the patient support apparatus; a height of the support surface; a charge state of a battery onboard patient support apparatus; a connection strength of the network transceiver to the healthcare facility computer network; an armed or disarmed state of the sensor; an armed or disarmed state of an onboard monitoring system; or a state of a power cable adapted to couple the patient support apparatus to a source of electrical power, or a state of a communication channel between the patient support apparatus and a communication outlet integrated into a wall of the healthcare facility.

In some aspects, the patient support apparatus further includes a memory in which the locations of each of the plurality of ultra-wideband transceivers are stored, and the controller is further adapted to use the stored locations of the plurality of ultra-wideband transceivers to determine whether the badge is positioned inside or outside of the volume of space.

The controller, in some aspects, is further adapted to use radio frequency (RF) communication between the plurality of ultra-wideband transceivers and a device to determine a position of the device relative to the patient support apparatus; to determine if the device is positioned inside or outside of a second volume of space; to automatically disassociate the device from the patient support apparatus if the device is positioned outside of the second volume of space; and to automatically associate the device with the patient support apparatus if the device is positioned inside the second volume of space.

In some aspects, the controller is further adapted to perform at least one of the following when the device is associated with the patient support apparatus: (a) forward data received from the device to the healthcare facility computer network, or (b) display data received from the device on a display of the patient support apparatus. The controller may be further adapted to perform neither (a) nor (b) when the device is disassociated from the patient support apparatus.

In some aspects, the controller is further adapted to use radio frequency (RF) communication between the plurality of ultra-wideband transceivers and a tag coupled to the patient to determine a position of the tag relative to the patient support apparatus, and to automatically send a location message to the healthcare facility computer network. The location message indicates a location of the patient.

The location message, in some aspects, indicates if the patient is positioned in or out of the patient support apparatus.

In some aspects, the location messages indicates if the patient is positioned out of a room in which the patient support apparatus is located.

The controller, in some aspects, is further adapted to use radio frequency (RF) communication between the plurality of ultra-wideband transceivers and a tag coupled to the patient to determine a position of the tag relative to the patient support apparatus, and to automatically determine a distance between the tag and the badge.

In some aspects, the controller is further adapted to send a message to the healthcare facility computer network if the distance exceeds a threshold.

According to another aspect of the present disclosure, a patient support apparatus is provided that includes a support surface adapted to support a patient; a plurality of ultra-wideband transceivers; a sensor adapted to detect an undesired state of the patient support apparatus; a network transceiver adapted to communicate with a healthcare facility computer network; and a controller. The controller is adapted to use radio frequency (RF) communication between the plurality of ultra-wideband transceivers and a badge to determine a position of the badge relative to the patient support apparatus, to use radio frequency (RF) communication between the plurality of ultra-wideband transceivers and a tag coupled to the patient to determine a position of the tag relative to the patient support apparatus, and to automatically determine a distance between the tag and the badge.

In another aspect of the present disclosure, the controller is further adapted to send a message to the healthcare facility computer network if the distance between the patient tag and caregiver badge exceeds a threshold.

In some aspects, the controller is further adapted to vary the threshold based on a condition of the patient.

The condition, in some aspects, is a fall risk assessment that exceeds a predetermined value.

In some aspects, the patient support apparatus further includes a sensor adapted to detect an undesired state of the patient support apparatus; wherein the controller is further adapted to issue an alert if the badge is positioned outside of the volume of space while the undesired state exists and the distance is less than the threshold; and to stop the alert if the badge is positioned inside the volume of space while the undesired state exists and the distance is less than the threshold.

In some aspects, the controller is configured to stop the alert for as long as the distance is less than the threshold and the undesired state continues to exist.

The sensor, in some aspects, is adapted to detect an exit of the patient from the patient support apparatus, and the undesired state is the patient exiting from the patient support apparatus.

In some aspects, the patient support apparatus further includes a plurality of siderails movable between raised and lowered positions, and the sensor is adapted to detect a position of at least one of the siderails, and the undesired state is the at least one of the siderails being moved to its lowered position.

According to another aspect of the present disclosure, a patient support apparatus system is provided that includes patient support apparatus and an offboard device. The patient support apparatus includes a support surface adapted to support a patient; a plurality of ultra-wideband transceivers; a network transceiver adapted to communicate with the off-board device; and a controller. The controller is adapted to use radio frequency (RF) communication between the plurality of ultra-wideband transceivers and a badge worn by a user to determine a position of the badge relative to the patient support apparatus; to receive a badge identifier from the badge; to determine if the badge is positioned inside or outside of a volume of space; to transmit the badge identifier to the offboard device using the network transceiver if the badge is inside the volume of space; and to transmit status data regarding the patient support apparatus to the offboard device. The offboard device is adapted to display a dashboard screen that includes the following: an identity of a room in which the patient support apparatus is located, an indicator indicating if the badge is inside the volume of space, and the status data.

According to another aspects of the present disclosure, the offboard device is adapted to display a room symbol corresponding to the room on the dashboard screen, and to display the indicator and the status data inside of the room symbol.

The status data, in some aspects, includes at least one of the following: a status of a siderail on the patient support apparatus; a status of a brake on the patient support apparatus; a height of the support surface; a charge state of a battery onboard patient support apparatus; a connection strength of the network transceiver to the healthcare facility computer network; an armed or disarmed state of the sensor; an armed or disarmed state of an onboard monitoring system; or a state of a power cable adapted to couple the patient support apparatus to a source of electrical power; or a state of a communication channel between the patient support apparatus and a communication outlet integrated into a wall of the healthcare facility.

The offboard device, in some aspects, is one of a server, a smart phone, a tablet computer, a laptop computer, or a desktop computer.

The patient support apparatus, in some aspects, further includes a first control adapted to perform a first function when activated by the user, and the controller is further adapted to transmit a function identifier to the offboard device if the badge is positioned inside the volume of space when the first control is activated. The function identifier identifies the first function.

In some aspects, the first function is one or more of the following: changing a state of an exit detection system onboard the patient support apparatus; measuring a weight of the patient; performing a mattress therapy on the patient; changing a state of a mattress; changing a state of a brake onboard the patient support apparatus; changing a state of a siderail onboard the patient support apparatus; changing a height of the support surface; changing a reminder provided by the controller; zeroing a scale onboard the patient support apparatus; configuring a component onboard the patient support apparatus; changing a setting of the patient support apparatus; changing information stored in the patient support apparatus; or performing maintenance work on the patient support apparatus.

In some aspects, the offboard device is adapted to use the function identifier to display information about the first function on the dashboard screen.

According to yet another aspect of the present disclosure, a patient support apparatus system is provided that include a patient support apparatus and a server. The patient support apparatus includes a support surface adapted to support a patient; a plurality of ultra-wideband transceivers; a network transceiver adapted to communicate with a healthcare facility computer network; and a controller. The controller is adapted to use radio frequency (RF) communication between the plurality of ultra-wideband transceivers and a badge to determine a position of the badge relative to the patient support apparatus; to receive a badge identifier from the badge; to determine if the badge is positioned inside or outside of a volume of space; and, if the badge is positioned inside the volume of space, to transmit the badge identifier to the server. The server is adapted to perform the following: to receive notice of an event; to identify a particular type of healthcare workers who should be present at the patient support apparatus for the event; to use the badge identifier to determine if the particular type of healthcare worker is present at the patient support apparatus; and to issue an alert if the particular type of healthcare worker is absent from the patient support apparatus.

In some aspects of the present disclosure, the server is further adapted to issue the alert by sending a message to a selected healthcare worker of the particular type.

In some aspects, the server is configured to send the message to the selected healthcare worker by sending the message to a first badge associated with the selected healthcare worker.

The server, in some aspects, is configured to select the selected healthcare worker from amongst a plurality of healthcare workers of the particular type.

In some aspects, the server is configured to select the selected healthcare worker by selecting the closest healthcare worker of the particular type to the patient support apparatus.

In some aspects, the server is adapted to identify a second type of healthcare workers who should be present at the patient support apparatus for the event, to use the badge identifier to determine if the second type of healthcare worker is present at the patient support apparatus, and to issue a second alert if the second type of healthcare worker is absent from the patient support apparatus.

The server, in some aspects, is further adapted to issue the second alert by sending a second message to a second selected healthcare worker of the second type.

In some aspects, the server is configured to send the second message to the second selected healthcare worker by sending the second message to a second badge associated with the second selected healthcare worker.

The server, in some aspects, is adapted to determine a priority level of the event, and to use the priority level when selecting the selected healthcare worker.

In some aspects, if the priority level is above a first threshold, the server is configured to select the selected healthcare worker without regard to which healthcare worker is assigned to the patient in the patient support apparatus.

In some aspects, if the priority level is below the first threshold, the server is configured to select the selected healthcare worker from amongst healthcare workers who are assigned to the patient in the patient support apparatus.

In some aspects, if the priority level is below a second threshold less than the first threshold, the server is configured to select the selected healthcare worker without regard to which healthcare worker is assigned to the patient in the patient support apparatus.

The event, in some aspects, is one or more of the following: a cardiac arrest of the patient, a respiratory arrest of the patient, a patient fall, a patient exit from the patient support apparatus, a call from the patient, or an undesired state of the patient support apparatus.

The particular type of healthcare worker, in some aspects, is a doctor, a nurse, a physician's assistant, or another type of healthcare worker.

In some aspects, the server is further configured to identify a group of healthcare workers who should be present at the patient support apparatus for the event, to use the badge identifier to determine if the group of healthcare workers is actually present at the patient support apparatus, and to issue the alert if the group of healthcare workers is absent from the patient support apparatus.

In some aspects, the event is an event associated with a condition of the patient, and the patient support apparatus further includes a sensor adapted to detect an undesired state of the patient support apparatus, and the controller is further adapted to issue a second alert if the badge is positioned outside of the volume of space while the undesired state exists; and to stop the second alert if the badge is positioned inside the volume of space while the undesired state exists.

The controller, in some aspects, is configured to stop the second alert for as long as the badge remains inside the volume of space and the undesired state continues to exist.

The sensor, in some aspects, is adapted to detect an exit of the patient from the patient support apparatus, and the undesired state is the patient exiting from the patient support apparatus.

The patient support apparatus, in some aspects, further includes a plurality of siderails movable between raised and lowered positions, and wherein the sensor is adapted to detect a position of at least one of the siderails, and the undesired state is the at least one of the siderails being moved to its lowered position.

According to yet another aspect of the present disclosure, a patient support apparatus system is provided that includes a plurality of patient support apparatuses and a server. Each of the patient support apparatuses includes a support surface adapted to support a patient; a plurality of ultra-wideband transceivers; a network transceiver adapted to communicate with a healthcare facility computer network; and a controller. The controller is adapted to use radio frequency (RF) communication between the plurality of ultra-wideband transceivers and a badge to determine a position of the badge relative to the patient support apparatus. The controller is further adapted to receive a badge identifier from the badge and to transmit the badge identifier to the server. The server is adapted to receive badge identifiers of a plurality of badges from the plurality of patient support apparatuses, to receive an alert from an alerting one of the plurality of patient support apparatuses, and to determine relative positions of the plurality of badges to the alerting patient support apparatus.

According to another aspect of the present disclosure, the server is further adapted to determine a first set of badges corresponding to healthcare workers assigned to care for the patient in the alerting patient support apparatus.

In some aspects, the server is further adapted to determine which one of the plurality of badges in the first set of badges is positioned closest to the alerting patient support apparatus.

The server, in some aspects of the present disclosure, is further configured to automatically send an alert message to the badge in the first set of badges that is positioned closest to the alerting patient support apparatus.

In some aspects, the server is further adapted to determine a priority level of the alert.

The server, in some aspects, is further configured to determine a second set of badges corresponding to healthcare workers not assigned to care for the patient in the alerting patient support apparatus.

In some aspects, the server is further configured to determine if the priority level of the alert is above a first threshold, and if the priority level is above the first threshold, to determine which one of the plurality of badges out of both the first and second sets of badges is positioned closest to the alerting patient support apparatus.

In some aspects, each controller of the plurality of patient support apparatuses is further adapted to use RF communication between the plurality of ultra-wideband transceivers and a fixed locator to determine a position of the patient support apparatus relative to the fixed locator.

Each controller, in some aspects, is further adapted to receive a locator identifier from the fixed locator, to determine if the patient support apparatus is positioned within a threshold distance of the fixed locator, and to forward the locator identifier to the server via the network transceiver if the patient support apparatus is positioned within the threshold distance.

The server, in some aspects, is further configured to use the locator identifiers from the plurality of patient support apparatuses when determining which one of the plurality of badges in the first set of badges is positioned closest to the alerting patient support apparatus.

Each controller, in some aspects, is further adapted to use radio frequency (RF) communication between the plurality of ultra-wideband transceivers and a device to determine a position of the device relative to the patient support apparatus; to determine if the device is positioned inside or outside of a second volume of space; to automatically disassociate the device from the patient support apparatus if the device is positioned outside of the second volume of space; and to automatically associate the device with the patient support apparatus if the device is positioned inside the second volume of space.

In some aspects, each controller is further adapted to perform at least one of the following when the device is associated with the patient support apparatus: (a) forward data received from the device to the server, or (b) display data received from the device on a display of the patient support apparatus.

Each controller, in some aspects, is further adapted to perform neither (a) nor (b) when the device is disassociated from the patient support apparatus.

Each patient support apparatus, in some aspects, further includes a sensor adapted to detect an undesired state of the patient support apparatus, and the controller of each patient support apparatus is further adapted to send the alert to the server if the badge is positioned outside of the volume of space while the undesired state exists, and to not send the alert to the server if the badge is positioned inside the volume of space while the undesired state exists.

According to yet another aspect of the present disclosure, a patient support apparatus system is provided that includes a patient support apparatus, a server, and a fixed locator. The fixed locator is positioned adjacent an entrance to an area of a healthcare facility. The patient support apparatus includes a support surface adapted to support a patient, a plurality of ultra-wideband transceivers, and a controller. The controller is adapted to use radio frequency (RF) communication between the plurality of ultra-wideband transceivers and the fixed locator to determine a direction of movement of the patient support apparatus as the patient support apparatus moves past the fixed locator. The controller is further adapted to receive a location identifier from the fixed locator and to transmit both the location identifier and the direction of movement to the server. The server is adapted to use the location identifier and the direction of movement to determine whether the patient support apparatus is exiting from, or entering into, the area of the healthcare facility.

According to yet another aspect of the present disclosure, a patient support apparatus is provided. The patient support apparatus includes a support surface adapted to support a patient, a plurality of ultra-wideband transceivers, and a controller. The controller is adapted to use radio frequency (RF) communication between the plurality of ultra-wideband transceivers and a badge to determine a position of the badge relative to the patient support apparatus. The controller is further adapted to receive a badge identifier from the badge, to determine if a state of a component of the patient support apparatus changes, and to store in memory a log containing the badge identifier of any badge that was within a threshold distance of the patient support apparatus when the state of the component changed.

According to yet another aspect of the present disclosure, a patient support apparatus system is provided that includes a patient support apparatus and a server. A patient support apparatus system comprising a patient support apparatus and a server. The patient support apparatus comprises a support surface adapted to support a patient, a plurality of ultra-wideband transceivers, and a controller. The controller is adapted to use radio frequency (RF) communication between the plurality of ultra-wideband transceivers and a badge to determine a position of the badge relative to the patient support apparatus. The controller is further adapted to receive a badge identifier from the badge, to determine if a state of a component of the patient support apparatus changes, and to transmit to the server the badge identifier of any badge that was within a threshold distance of the patient support apparatus when the state of the component changed. The server is adapted to store in memory a log containing the badge identifier of any badge that was within a threshold distance of the patient support apparatus when the state of the component changed.

According to yet another aspect of the present disclosure, a patient support apparatus is provided that includes a support surface adapted to support a patient, a plurality of ultra-wideband transceivers, an actuator, and a controller. The actuator is adapted to move a component of the patient support apparatus through a movement path. The controller is adapted to use radio frequency (RF) communication between the plurality of ultra-wideband transceivers and a badge to determine a three-dimensional position of the badge relative to the patient support apparatus. The controller is further adapted to determine if the three-dimensional position of the badge is within a threshold distance of the movement path and to prevent movement of the component if the three-dimensional position of the badge is within the threshold distance of the movement path.

According to still other aspects of the present disclosure, the server is further adapted to record a time when the position of the patient support apparatus relative to the fixed locator is determined.

In some aspects, the server is further adapted to transmit data to a computer device in communication with the server, wherein the data indicates whether the patient support apparatus has exited from, or entered into, the area of the healthcare facility.

The area of the healthcare facility, in some aspects, is one of a wing, a department, a hallway, an elevator, or a floor.

The area of the healthcare facility, in some aspects, includes a plurality of entrances and each of the plurality of entrances includes a fixed locator positioned adjacent thereto.

The server, in some aspects, is configured to determine whether the patient support apparatus is moving into, or moving out of, the area of the healthcare facility whenever the patient support apparatus moves through any of the plurality of entrances.

In some aspects, the area of the healthcare facility is bounded by a set of entrances such that entry into the area is not possible without passing through an entrance in the set of entrances. In such cases, each entrance in the set of entrances includes a fixed locator, and the server is further configured to determine whether the patient support apparatus is moving into, or moving out of, the area of the healthcare facility whenever the patient support apparatus moves through any of the entrances in the set of entrances.

In some aspects, the patient support apparatus system includes a second fixed locator positioned adjacent a second entrance into a second area of the healthcare facility, and the controller is adapted to determine use radio frequency (RF) communication between the plurality of ultra-wideband transceivers and the second fixed locator to determine a second direction of movement of the patient support apparatus as the patient support apparatus moves past the second fixed locator. The controller is further adapted to receive a second location identifier from the second fixed locator and to transmit both the second location identifier and the second direction of movement to the server. The server is adapted to use the second location identifier and the second direction of movement to determine whether the patient support apparatus is exiting from, or entering into, the second area of the healthcare facility.

In some aspects, the second area of the healthcare facility includes a plurality of second entrances, and each of the plurality of second entrances includes a fixed locator positioned adjacent thereto.

The server, in some aspects, is further configured to determine whether the patient support apparatus is moving into, or moving out of, the second area of the healthcare facility whenever the patient support apparatus moves through any of the plurality of second entrances.

The second area of the healthcare facility, in some aspects, is bounded by a second set of entrances such that entry into the second area is not possible without passing through an entrance in the second set of entrances. In such cases, each entrance in the second set of entrances includes a fixed locator, and the server is further configured to determine whether the patient support apparatus is moving into, or moving out of, the second area of the healthcare facility whenever the patient support apparatus moves through any of the entrances in the second set of entrances.

In some aspects, the controller is further adapted to use radio frequency (RF) communication between the plurality of ultra-wideband transceivers and a room locator positioned within a room of the healthcare facility to determine a position of the patient support apparatus relative to the room locator. The controller is further adapted to receive a room identifier from the room locator and to transmit the room identifier to the server. The server is adapted to use the room identifier to determine a room in which the patient support apparatus is currently positioned.

In some aspects, the controller is adapted to not use communication with the room locator to determine whether the patient support apparatus is exiting from, or entering into, the room.

The area of the healthcare facility, in some aspects, does not refer to a room.

The server, in some aspects, includes data indicating whether the fixed locator is positioned adjacent an elevator or not.

The server, in some aspects, is adapted to determine what floor within the healthcare facility the patient support apparatus is currently located based on data received from a plurality of fixed locators positioned adjacent an elevator in the healthcare facility.

The controller, in some aspects, is further adapted to determine a position of the patient support apparatus relative to the fixed locator, and at least one of the controller or the server is further adapted to use the relative position of the patient support apparatus to the fixed locator to determine if the patient support apparatus is positioned on the same floor as the fixed locator or not.

In some aspects, the patient support apparatus system also includes a plurality of fixed locators, and the server includes data identifying a first set and a second set of the plurality of fixed locators. The first set of the plurality of fixed locators is positioned at entrances to areas of the healthcare facility and the second set of the plurality of fixed locators is positioned within rooms.

The server, in some aspects, is adapted to use data from a fixed locator within the first set of the plurality of fixed locators to determine in which non-room area of the healthcare facility the patient support apparatus is currently located, and to use data from a fixed locator within the second set of the plurality of fixed locators to determine in which room the patient support apparatus is currently located.

In some aspects, the controller is further adapted to use radio frequency (RF) communication between the plurality of ultra-wideband transceivers and a badge to determine a position of the badge relative to the patient support apparatus, to receive a badge identifier from the badge, to determine if a state of a component of the patient support apparatus changes, and to perform at least one of the following: (a) store in memory a log containing the badge identifier of any badge that was within a threshold distance of the patient support apparatus when the state of the component changed; or (b) transmit to the server the badge identifier of any badge that was within a threshold distance of the patient support apparatus when the state of the component changed. The server is adapted to store in memory a log containing the badge identifier of any badge that was within a threshold distance of the patient support apparatus when the state of the component changed.

In some aspects, the controller is further adapted to use RF communication between the plurality of ultra-wideband transceivers and an equipment tag to determine a position of the equipment tag relative to the patient support apparatus, to receive an equipment tag identifier from the equipment tag, and to perform at least one of the following: (a) store in the log the equipment tag identifier of any equipment tag that was within a second threshold distance of the patient support apparatus when the state of the component changed; or (b) transmit to a server the equipment tag identifier of any equipment tag that was within the second threshold distance of the patient support apparatus when the state of the component changed. The server is adapted to store in the log the equipment tag identifier of any equipment tag that was within the second threshold distance of the patient support apparatus when the state of the component changed.

In some aspects, the second threshold distance is different from the threshold distance, while in other aspects, the second threshold distance may be the same as the threshold distance.

In some aspects, the controller is adapted to determine the position of the badge relative to the patient support apparatus in three dimensions, and perform at least one of the following: (a) to store in the log the three-dimensional position of the badge if the badge was within the threshold distance of the patient support apparatus when the state of the component changed; or (b) transmit to the server the three-dimensional position of any badge that was within the threshold distance of the patient support apparatus when the state of the component changed.

In some aspects, the controller is adapted to determine the position of the equipment tag relative to the patient support apparatus in three dimensions, and to perform at least one of the following: (a) store in the log the three-dimensional position of the equipment tag if the equipment tag was within the second threshold distance of the patient support apparatus when the state of the component changed; or (b) transmit to the server the three-dimensional position of any equipment tag that was within the second threshold distance of the patient support apparatus when the state of the component changed.

In some aspects, the component of the patient support apparatus is one of the following: a brake, an actuator, a siderail, a plug, a cable, the support surface, an exit detection system, a monitoring system, a mattress, any of the plurality of ultra-wideband transceivers, a litter frame, a scale system, a control panel, or a propulsion system.

In some aspects, the controller is further adapted to use the badge identifier to determine an identity of a person associated with the badge.

The controller, in some aspects, is further adapted to use the equipment tag identifier to determine an identity of a piece of equipment associated with the equipment tag.

The controller, in some aspects, is further adapted to select a specific value of the threshold distance based on which specific component of the patient support apparatus changes.

The patient support apparatus, in some aspects, further includes an actuator adapted to move a component of the patient support apparatus through a movement path, and the controller is adapted to use radio frequency (RF) communication between the plurality of ultra-wideband transceivers and a badge to determine a three-dimensional position of the badge relative to the patient support apparatus. The controller is further adapted to determine if the three-dimensional position of the badge is within a stored distance of the movement path and to prevent movement of the component if the three-dimensional position of the badge is within the stored distance of the movement path.

The controller, in some aspects, is adapted to retrieve data indicating three-dimensional positions of the plurality of ultra-wideband transceivers relative to at least one three-dimensional point of the movement path.

The component, in some aspects, is a litter frame and the actuator is a lift adapted to change a height of at least a portion of the support surface.

The component, in some aspects, is a portion of the support surface and the actuator is adapted to change an 202 of the portion of the support surface.

In some aspects, the controller is adapted to prevent movement of the component in a first direction but not a second direction if the three-dimensional position of the badge is within the stored distance of the movement path.

The first direction, in some aspects, is a direction that moves the component closer to the badge and the second direction is a direction that moves the component farther from the badge.

The controller, in some aspects, is further adapted to use radio frequency (RF) communication between the plurality of ultra-wideband transceivers and a smart phone equipped with a UWB transceiver to determine a three-dimensional position of the smart phone relative to the patient support apparatus. The controller is further adapted to determine if the three-dimensional position of the smart phone is within a second stored distance of the movement path and to prevent movement of the component if the three-dimensional position of the smart phone is within the second stored distance of the movement path.

In some aspects, the controller is adapted to determine the three-dimensional position of the smart phone relative to the patient support apparatus without pairing with the smart phone.

The controller, in some aspects, is adapted to prevent movement of the component in a first direction but not a second direction if the three-dimensional position of the smart phone is within the stored distance of the movement path. The first direction is a direction that moves the component closer to the smart phone and the second direction is a direction that moves the component farther from the smart phone.

In some aspects, the controller is further adapted to use radio frequency (RF) communication between the plurality of ultra-wideband transceivers and an equipment tag to determine a three-dimensional position of the equipment tag relative to the patient support apparatus. The controller is further adapted to determine if the three-dimensional position of the equipment tag is within a second stored distance of the movement path and to prevent movement of the component if the three-dimensional position of the equipment tag is within the second stored distance of the movement path.

In some aspects, the controller is adapted to prevent movement of the component in a first direction but not a second direction if the three-dimensional position of the equipment tag is within the second stored distance of the movement path. The first direction is a direction that moves the component closer to the equipment tag and the second direction is a direction that moves the equipment tag farther from the equipment tag.

In some aspects, the controller is further adapted to use RF communication between the plurality of ultra-wideband transceivers and a smart phone equipped with a UWB transceiver to determine a position of the smart phone relative to the patient support apparatus, to receive a smart phone identifier from the smart phone, and to store in the log the smart phone identifier of any smart phone that was within a third threshold distance of the patient support apparatus when the state of the component changed.

The controller, in some aspects, is adapted to determine the position of the smart phone relative to the patient support apparatus in three dimensions, and to store in the log the three-dimensional position of the smart phone if the smart phone was within the third threshold distance of the patient support apparatus when the state of the component changed.

The controller, in some aspects, is adapted to transmit the log to a server, wherein the server is further adapted to transmit the log to a computer device in communication with the server.

In some aspects, the controller is further adapted to store in the log a time at which the state of the component changed.

In some aspects, the server is adapted to determine what floor within the healthcare facility the patient support apparatus is currently located based on data received from a plurality of fixed locators positioned adjacent an elevator in the healthcare facility.

The controller, in some aspects, is further adapted to determine a position of the patient support apparatus relative to the fixed locator, and at least one of the controller or the server is further adapted to use the relative position of the patient support apparatus to the fixed locator to determine if the patient support apparatus is positioned on the same floor as the fixed locator or not.

In some aspects, the controller is further adapted to use radio frequency (RF) communication between the plurality of ultra-wideband transceivers and all badges that are within communication range of the ultra-wideband transceivers to determine a position of all such in-range badges relative to the patient support apparatus, to receive badge identifiers from all of the in-range badges, and to transmit to the server the badge identifiers of all of the in-range badges when the state of the component changed. The server is further adapted to store in the log the badge identifiers from all of the in-range badges when the state of the component changed.

In some aspects, the controller is further adapted to use radio frequency (RF) communication between the plurality of ultra-wideband transceivers and all equipment tags that are within communication range of the ultra-wideband transceivers to determine a position of all such in-range equipment tags relative to the patient support apparatus, to receive equipment tag identifiers from all of the in-range equipment tags, and to transmit to the server the equipment tag identifiers of all of the in-range equipment tags when the state of the component changed. The server is further adapted to store in the log the equipment tag identifiers from all of the in-range equipment tags when the state of the component changed.

Before the various aspects of the disclosure are explained in detail, it is to be understood that the claims are not to be limited to the details of operation or to the details of construction and the arrangement of the components set forth in the following description or illustrated in the drawings. The aspects described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus according to a first aspect of the present disclosure;

FIG. 2 is a plan view of an illustrative caregiver control panel of the patient support apparatus of FIG. 1;

FIG. 3 is a plan view of an illustrative patient control panel of the patient support apparatus of FIG. 1;

FIG. 4 is a perspective view of a patient support apparatus system of the present disclosure showing the patient support apparatus, a device, a display device, a caregiver with a badge, and a linked locator unit that is used for automatically detecting the location of a patient support apparatus;

FIG. 5 is a block diagram of the patient support apparatus system of FIG. 4;

FIG. 6 is a plan view of an illustrative healthcare facility area, a patient support apparatus, a plurality of caregiver badges, a device, a plurality of space volumes, and a plurality of wall units;

FIG. 7 is a block diagram of an unlinked locator unit, the healthcare facility network, and a caregiver badge;

FIG. 8 is a diagram of the patient support apparatus system of FIG. 4 showing more details of some of the data contained in a plurality of servers that may be resident in the healthcare facility;

FIG. 9 is an example of a dashboard screen that may be displayed on a remote electronic device that shows the status of multiple patient support apparatuses, medical devices, and activities performed by caregivers, and other information;

FIG. 10 is an example of a log of events that may be detected by the patient support apparatus;

FIG. 11 is a diagram of an illustrative hallway in which movement of the patient support apparatus into, or out of, a particular area is detected;

FIG. 12 is an illustrative floor plan of a healthcare facility in which movement of patient support apparatuses into, or out of, one or more areas may be detected; and

FIG. 13 is a perspective view of a pair of patient support apparatuses illustrating examples of movement paths.

### DETAILED DESCRIPTION OF THE EMBODIMENTS

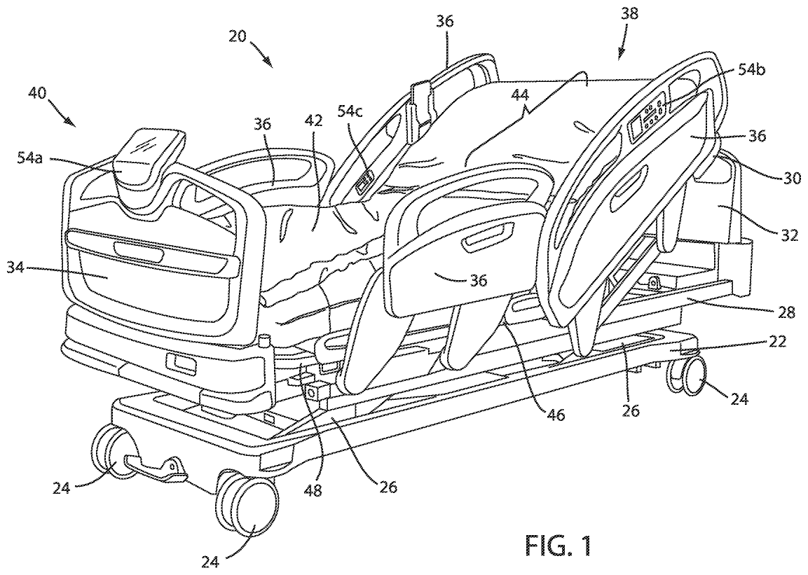

An illustrative patient support apparatus 20 according to an embodiment of the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a recliner, or any other structure capable of supporting a patient in a healthcare environment.

In general, patient support apparatus 20 includes a base 22 having a plurality of wheels 24, a pair of lifts 26 supported on the base 22, a litter frame 28 supported on the lifts 26, and a support deck 30 supported on the litter frame 28. Patient support apparatus 20 further includes a headboard 32, a footboard 34 and a plurality of siderails 36. Siderails 36 are all shown in a raised position in FIG. 1 but are each individually movable to a lower position in which ingress into, and egress out of, patient support apparatus 20 is not obstructed by the lowered siderails 36.

Lifts 26 are adapted to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted, to place the litter frame 28 in a flat or horizontal orientation, a Trendelenburg orientation, or a reverse Trendelenburg orientation. That is, litter frame 28 includes a head end 38 and a foot end 40, each of whose height can be independently adjusted by the nearest lift 26. Patient support apparatus 20 is designed so that when an occupant lies thereon, his or her head will be positioned adjacent head end 38 and his or her feet will be positioned adjacent foot end 40.

Litter frame 28 provides a structure for supporting support deck 30, the headboard 32, footboard 34, and siderails 36. Support deck 30 provides a support surface for a mattress 42, or other soft cushion, so that a person may lie and/or sit thereon. In some embodiments, the mattress 42 includes one or more inflatable bladders that are controllable via a blower, or other source of pressurized air. In at least one embodiment, the inflation of the bladders of the mattress 42 is controllable via electronics built into patient support apparatus 20. In one such embodiments, mattress 42 may take on any of the functions and/or structures of any of the mattresses disclosed in commonly assigned U.S. Pat. No. 9,468,307 issued Oct. 18, 2016, to inventors Patrick Lafleche et al., the complete disclosure of which is incorporated herein by reference. Still other types of mattresses may be used.

Support deck 30 is made of a plurality of sections, some of which are pivotable about generally horizontal pivot axes. In the embodiment shown in FIG. 1, support deck 30 includes at least a head section 44, a thigh section 46, and a foot section 48, all of which are positioned underneath mattress 42 and which generally form flat surfaces for supporting mattress 42. Head section 44, which is also sometimes referred to as a Fowler section, is pivotable about a generally horizontal pivot axis between a generally horizontal orientation (not shown in FIG. 1) and a plurality of raised positions (one of which is shown in FIG. 1). Thigh section 46 and foot section 48 may also be pivotable about generally horizontal pivot axes.

In some embodiments, patient support apparatus 20 may be modified from what is shown to include one or more components adapted to allow the user to extend the width and/or length of patient support deck 30, thereby allowing patient support apparatus 20 to accommodate patients of varying sizes. When so modified, the width of deck 30 may be adjusted sideways and/or lengthwise in increments or otherwise.

As used herein, the term “longitudinal” refers to a direction parallel to an axis between the head end 38 and the foot end 40. The terms “transverse” or “lateral” refer to a direction perpendicular to the longitudinal direction and parallel to a surface on which the patient support apparatus 20 rests.

It will be understood by those skilled in the art that patient support apparatus 20 can be designed with other types of mechanical constructions that are different from what is shown in the attached drawings, such as, but not limited to, the construction described in commonly assigned, U.S. Pat. No. 10,130,536 to Roussy et al., entitled PATIENT SUPPORT USABLE WITH BARIATRIC PATIENTS, the complete disclosure of which is incorporated herein by reference. In another embodiment, the mechanical construction of patient support apparatus 20 may include the same, or nearly the same, structures as the Model 3002 S3 bed manufactured and sold by Stryker Corporation of Kalamazoo, Michigan. This construction is described in greater detail in the Stryker Maintenance Manual for the MedSurg Bed, Model 3002 S3, published in 2010 by Stryker Corporation of Kalamazoo, Michigan, the complete disclosure of which is incorporated herein by reference. In still another embodiment, the mechanical construction of patient support apparatus 20 may include the same, or nearly the same, structure as the Model 3009 Procuity MedSurg bed manufactured and sold by Stryker Corporation of Kalamazoo, Michigan. This construction is described in greater detail in the Stryker Maintenance Manual for the 3009 Procuity MedSurg bed (publication 3009-009-002, Rev. A.0), published in 2020 by Stryker Corporation of Kalamazoo, Michigan.

It will be understood by those skilled in the art that patient support apparatus 20 can be designed with still other types of mechanical constructions, such as, but not limited to, those described in commonly assigned, U.S. Pat. No. 7,690,59 issued Apr. 6, 2010, to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The overall mechanical construction of patient support apparatus 20 may also take on still other forms different from what is disclosed in the aforementioned references provided the patient support apparatus includes one or more of the functions, features, and/or structures discussed in greater detail below.

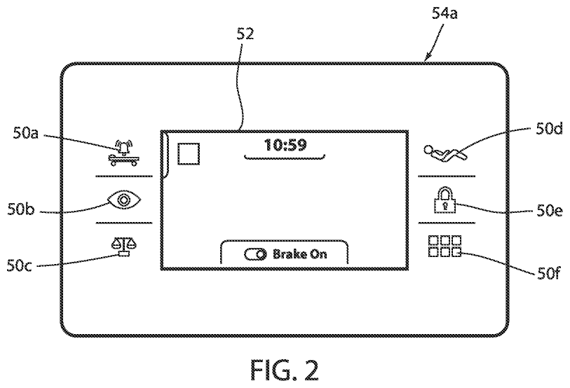

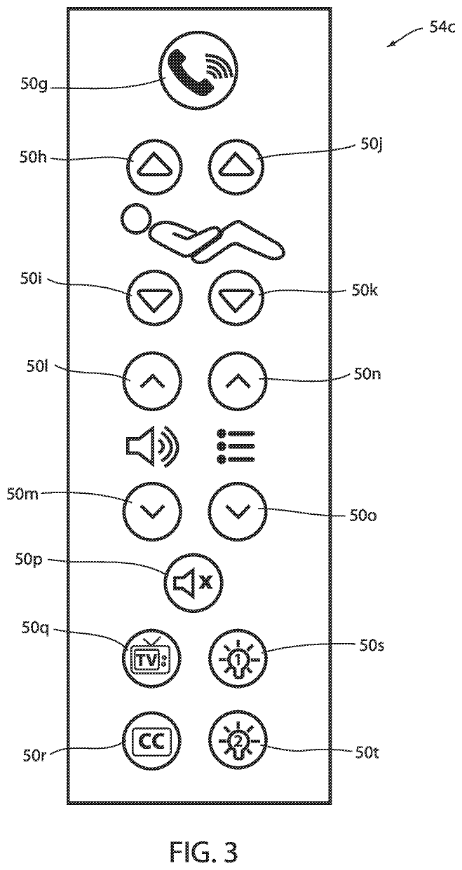

Patient support apparatus 20 further includes a plurality of control panels 54 that enable a user of patient support apparatus 20, such as a patient and/or an associated caregiver, to control one or more aspects of patient support apparatus 20. In the embodiment shown in FIG. 1, patient support apparatus 20 includes a footboard control panel 54a, a pair of outer siderail control panels 54b (only one of which is visible), and a pair of inner siderail control panels 54c (only one of which is visible). Footboard control panel 54a and outer siderail control panels 54b are intended to be used by caregivers, or other authorized personnel, while inner siderail control panels 54c are intended to be used by the patient associated with patient support apparatus 20. Each of the control panels 54 includes a plurality of controls 50 (see, e.g. FIGS. 2-3), although each control panel 54 does not necessarily include the same controls and/or functionality.

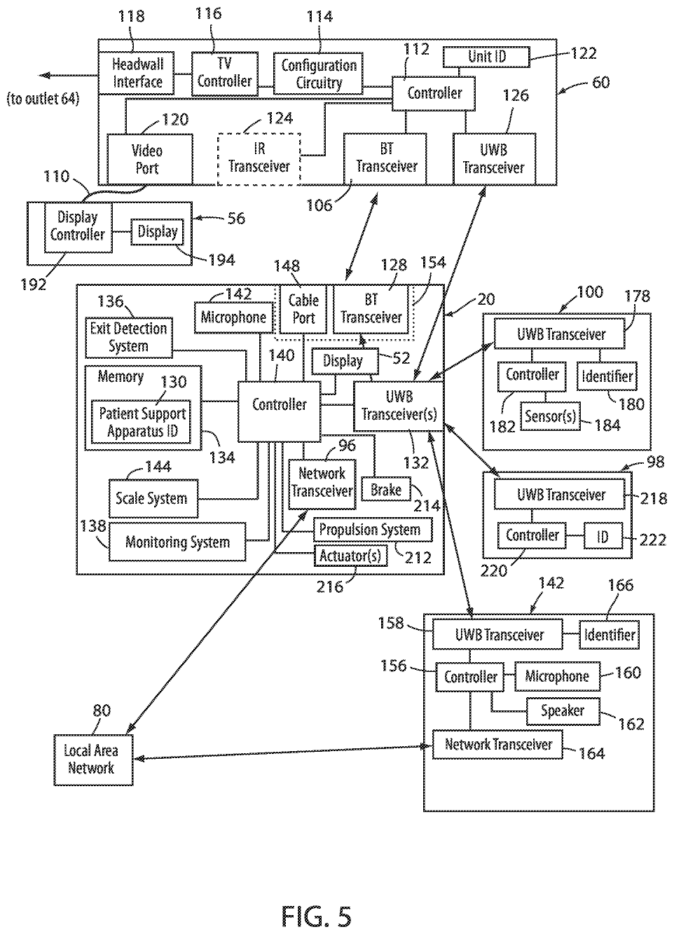

Among other functions, controls 50 of control panel 54a allow a user to control one or more of the following: change a height of support deck 30; raise or lower head section 44; activate and deactivate a brake for wheels 24; arm and disarm an exit detection system 136 and/or an onboard monitoring system 138 (FIG. 5); change various settings on patient support apparatus 20; view the current location of the patient support apparatus 20 as determined by the location detection system discussed herein; view what devices, such as, but not limited to, medical devices, exercise devices, nurse call devices, caregiver badges, etc. that the patient support apparatus 20 has associated itself with; and perform other actions. One or both of the inner siderail control panels 54c also include at least one control that enables a patient to call a remotely located nurse (or other caregiver). In addition to the nurse call control, one or both of the inner siderail control panels 54c also include one or more controls for controlling one or more features of one or more room devices positioned within the same room as the patient support apparatus 20. As will be described in more detail below, such room devices include, but are not necessarily limited to, a television, a reading light, and a room light. With respect to the television, the features that may be controllable by one or more controls 50 on control panel 54c include, but are not limited to, the volume, the channel, the closed-captioning, and/or the power state of the television. With respect to the room and/or night lights, the features that may be controlled by one or more controls 50 on control panel 54c include the on/off state and/or the brightness level of these lights.

Control panel 54a includes a display 52 (FIG. 2) configured to display a plurality of different screens thereon. Surrounding display 52 are a plurality of navigation controls 50a-f that, when activated, cause the display 52 to display different screens on display 52. More specifically, when a user presses navigation control 50a, control panel 54a displays an exit detection control screen on display 52 that includes one or more icons that, when touched, control an onboard exit detection system 136 (FIG. 5). The exit detection system 136 is as adapted to issue an alert when a patient exits from patient support apparatus 20. Exit detection system 136 may include any of the same features and functions as, and/or may be constructed in any of the same manners as, the exit detection system disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, the complete disclosure of which is incorporated herein by reference. Other types of exit detection systems may be included within patient support apparatus 20.

When a user presses navigation control 50b (FIG. 2), control panel 54 displays a monitoring control screen that includes a plurality of control icons that, when touched, control the onboard monitoring system 138 (FIG. 5) built into patient support apparatus 20. The onboard monitoring system 138 alerts the caregiver through a unified indicator, such as a light or a plurality of lights controlled in a unified manner, when any one or more of a plurality of settings on patient support apparatus 20 are in an undesired state, and uses that same unified indicator to indicate when all of the plurality of settings are in their respective desired states. Stated alternatively, monitoring system 138, when armed, monitors a plurality of conditions of patient support apparatus 20 (such as, but not limited to, any one or more of the following: brake status, siderail position, litter frame height, exit detection system 136, A/C cord status, nurse call cable status, etc.) and issues an alert if any one of those conditions are in an undesired state. Further details of one type of monitoring system that may be built into patient support apparatus 20 are disclosed in commonly assigned U.S. patent application Ser. No. 62/864,638 filed Jun. 21, 2019, by inventors Kurosh Nahavandi et al. and entitled PATIENT SUPPORT APPARATUS WITH CAREGIVER REMINDERS, as well as commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosures of both of which are incorporated herein by reference. Other types of monitoring systems may be included within patient support apparatus 20.

When a user presses navigation control 50c, control panel 54a displays a scale control screen that includes a plurality of control icons that, when touched, control a scale system 144 (FIG. 5) of patient support apparatus 20. Such a scale system 144 may include any of the same features and functions as, and/or may be constructed in any of the same manners as, the scale systems disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, and U.S. patent application Ser. No. 62/885,954 filed Aug. 13, 2019, by inventors Kurosh Nahavandi et al. and entitled PATIENT SUPPORT APPARATUS WITH EQUIPMENT WEIGHT LOG, the complete disclosures of both of which are incorporated herein by reference. The scale system may utilize the same force sensors and/or other components that are utilized by the exit detection system 136, or it may utilize one or more different sensors and/or other components. Other scale systems besides those mentioned above in the '254 and '954 applications may alternatively be included within patient support apparatus 20.

When a user presses navigation control 50d, control panel 54 displays a motion control screen that includes a plurality of control icons that, when touched, control the movement of various components of patient support apparatus 20, such as, but not limited to, the height of litter frame 28 and the pivoting of head section 44. In some embodiments, the motion control screen displayed on display 52 in response to pressing control 50d may be the same as, or similar to, the position control screen 216 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of motion control screens may be included on patient support apparatus 20.

When a user presses navigation control 50e, control panel 54a displays a motion lock control screen that includes a plurality of control icons that, when touched, control one or more motion lockout functions of patient support apparatus 20. Such motion lockout functions typically include the ability for a caregiver to use control panel 54a to lock out one or more of the motion controls 50 of the patient control panels 54c such that the patient is not able to use those controls 50 on control panels 54c to control the movement of one or more components of patient support apparatus 20. The motion lockout screen may include any of the features and functions as, and/or may be constructed in any of the same manners as, the motion lockout features, functions, and constructions disclosed in commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosure of which is incorporated herein by reference. Other types of motion lockouts may be included within patient support apparatus 20.

When a user presses on navigation control 50f, control panel 54a displays a menu screen that includes a plurality of menu icons that, when touched, bring up one or more additional screens for controlling and/or viewing one or more other aspects of patient support apparatus 20. Such other aspects include, but are not limited to, displaying information about one or more devices that are currently associated with patient support apparatus 20, diagnostic and/or service information for patient support apparatus 20, mattress control and/or status information, configuration settings, location information, and other settings and/or information. One example of such a menu screen is shown in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of menus and/or settings may be included within patient support apparatus 20. In at least one embodiment, utilization of navigation control 50f allows a user to navigate to a screen that enables a user to see which devices, if any, are currently associated with patient support apparatus 20. As will be discussed in greater detail below, patient support apparatus 20 includes an onboard locating system that is adapted to automatically determine the relative position of one or more devices with respect to patient support apparatus 20 and, in some instances, automatically associate and/or disassociate those devices with and/or from patient support apparatus 20 (and/or the patient assigned to patient support apparatus 20) depending upon the proximity of the device to patient support apparatus 20. Further details of this locating system are provided below.

For all of the navigation controls 50a-f (FIG. 2), screens other than the ones specifically mentioned above may be displayed on display 52 in other embodiments of patient support apparatus 20 in response to a user pressing these controls. Thus, it will be understood that the specific screens mentioned above are merely representative of the types of screens that are displayable on display 52 in response to a user pressing on one or more of navigation controls 50a-f. It will also be understood that, although navigation controls 50a-f have all been illustrated in the accompanying drawings as dedicated controls that are positioned adjacent display 52, any one or more of these controls 50a-f could alternatively be touchscreen controls that are displayed at one or more locations on display 52. Still further, although controls 50a-f have been shown herein as buttons, it will be understood that any of controls 50a-f could also, or alternatively, be switches, dials, or other types of non-button controls. Additionally, patient support apparatus 20 may be modified to include additional, fewer, and/or different navigation controls from the navigation controls 50a-f shown in FIG. 2.

FIG. 3 illustrates one example of a patient control panel 54c that may be incorporated into patient support apparatus 20 and positioned at a location on patient support apparatus 20 that is convenient for a patient to access while supported on support deck 30, such as on an interior side of one of the siderails 36. Control panel 54c includes a plurality of controls 50g-t that are intended to be operated by a patient. A nurse call control 50g, when pressed by the patient, sends a signal to a nurse call system requesting that a remotely positioned nurse talk to the patient. A Fowler-up control 50h, when pressed by the patient, causes a motorized actuator onboard patient support apparatus 20 to raise Fowler section 44 upwardly. A Fowler-down control 50i, when pressed by the patient, causes the motorized actuator to lower Fowler section 44 downwardly. A gatch-up control 50j, when pressed by the patient, causes another motorized actuator to raise a knee section of support deck 30, while a gatch-down control 50k causes the motorized actuator to lower the knee section of support deck 30. The knee section may refer to the joint that couples thigh section 46 to foot section 48.

A volume-up control 50l, when pressed by the patient, causes patient support apparatus 20 to send a signal to an in-room television instructing it to increase its volume, while a volume down control 50m, when pressed, causes patient support apparatus 20 to send a signal to the television instructing it to decrease its volume. A channel-up control 50n, when pressed by the patient, causes patient support apparatus 20 to send a signal to the television instructing it to increase the channel number, while a channel-down control 500, when pressed, causes patient support apparatus 20 to send a signal to the television instructing it to decrease the channel number.

A mute control 50p, when pressed, causes patient support apparatus 20 to send a signal to the television instructing it to either mute itself or unmute itself, depending upon whether the television is currently muted or unmuted. In other words, mute control 50p is a toggle control that alternatingly sends mute and unmute commands to the television when it is pressed.

Power control 50q is a toggle control that, when pressed, sends a signal to the television to either turn on or turn off, depending upon the television's current power status. Closed-captioning control 50r is another toggle control that, when pressed, sends a signal to the television to either turn on its closed-captioning feature or to turn off its closed captioning feature, depending upon whether the closed-captioning feature is currently on or off.

Control 50s is a toggle control that, when pressed, sends a signal to a first light to either turn on or turn off, depending upon the current state of that first light. Control 50t is another toggle control that, when pressed, sends a signal to a second light to either turn on or turn off, depending upon the current state of that second light. In some embodiments, the first light is a reading light and the second light is a room light, both of which are positioned off-board the patient support apparatus 20.

It will be understood that not only the number of controls 50 on control panel 54c, but also the functions of the controls 50 on control panel 54c, the layout of the controls 50 on control panel 54c, and/or other aspects of control panel 54c may be modified from what is shown in FIG. 3. In some embodiments, control panel 54c is implemented on a pendant controller that includes a cable that is plugged into a port on patient support apparatus 20. In other embodiments, one or more of the controls 50 of control panel 54c may be omitted, augmented, and/or split amongst other controls panels and/or locations. Still other manners of implementing control panel 54c are also possible.

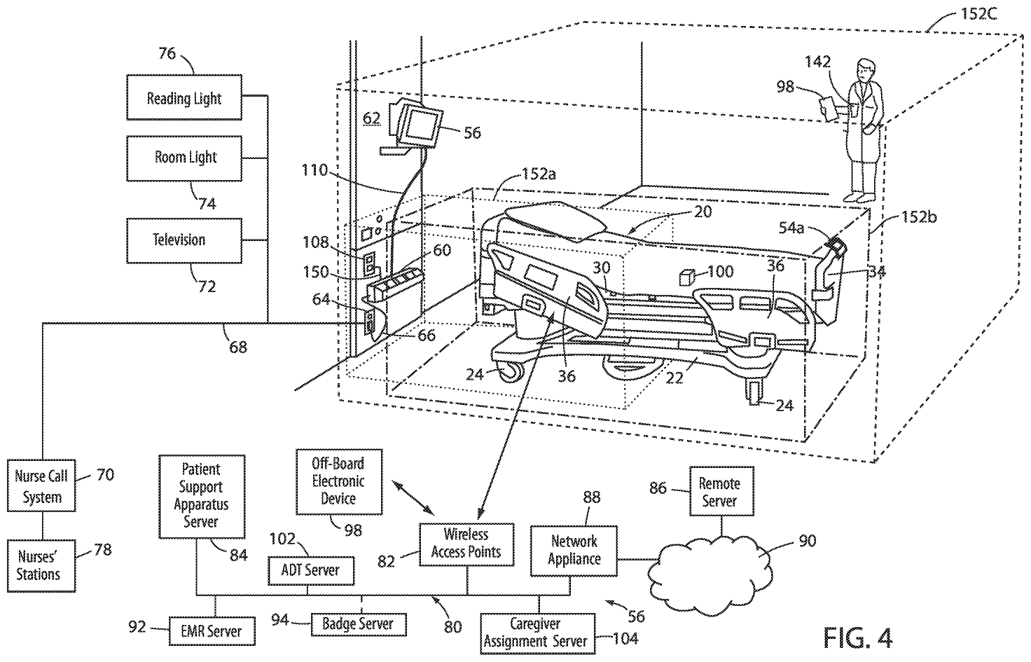

FIG. 4 illustrates patient support apparatus 20 positioned within a room 58 of a healthcare facility. FIG. 4 also illustrates additional items that may be present in a healthcare facility and which patient support apparatus 20 is configured to communicate with, including, but not limited to, a locator unit 60, a conventional local area network 80 of the healthcare facility, a caregiver badge 142, and one or more other devices 100 used during the care of a patient. Locator units 60 are positioned at known and fixed locations within the healthcare facility in which patient support apparatus 20 is positioned. Locator units 60 function as fixed locators. That is, locator units 60 communicate with patient support apparatuses 20 and share information with them that allows the location of the patient support apparatuses 20 to be determined.

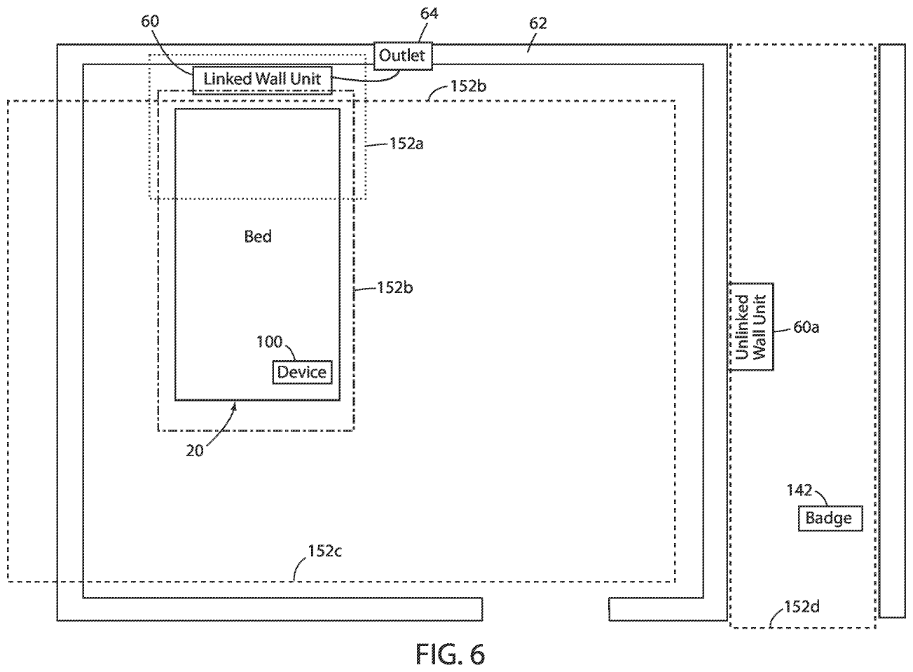

In some embodiments, patient support apparatus 20 is configured to be able to communicate with at least two different types of locator units 60: linked locator units 60 and unlinked locator units 60a. One example of a linked locator unit 60 is shown in FIG. 4. One example of an unlinked locator unit 60a is shown in FIG. 6. Other examples of unlinked locator units 60a are shown (and referred to as unlinked locator units 60b) in commonly assigned U.S. patent application Ser. No. 63/306,279 filed Feb. 3, 2022, by inventors Madhu Sandeep Thota et al. and entitled COMMUNICATION SYSTEM FOR PATIENT SUPPORT APPARATUSES, the complete disclosure of which is incorporated herein by reference. Patient support apparatus 20 is configured to communicate with the unlinked locator units 60b described in the aforementioned '279 application and to perform any one or more of the functions described therein that utilize such unlinked locator units and/or information provided by such unlinked locator units. Unless explicitly stated otherwise, all references herein to “locator units 60” without the term “linked” or “unlinked” in the reference will refer to both linked and unlinked locator units 60.

Linked locator units 60 are communicatively linked to a conventional communication outlet 64 and are adapted to provide location information to patient support apparatus 20. Linked locator units 60 are also adapted to serve as a communication conduit for routing wireless communications between patient support apparatus 20 and one or more devices and/or systems that are communicatively coupled to communication outlet 64 (e.g. room devices 72, 74, 76, and/or nurse call system 70, FIG. 4). In general, linked locator units 60 are typically positioned in patient rooms of the healthcare facility where one or more communication outlets 64 are typically present.

As shown in FIG. 4, linked locator unit 60 is adapted to be mounted to a wall 62, such as a headwall of a patient room 58 within the healthcare facility. The headwall of a conventional healthcare facility room 58 typically includes a conventional communications outlet 64 physically integrated therein. Communications outlet 64 is adapted to receive a nurse call cable 66 that physically connects at its other end either to patient support apparatus 20 (not shown) or to linked locator unit 60 (shown in FIG. 4). In many healthcare facilities, communication outlet 64 includes a 37-pin connector, although other types of connectors are often found in certain healthcare facilities. As will be discussed in greater detail below, linked locator unit 60 and nurse call cable 66 allow patient support apparatus 20 to communicate with a nurse call system, and one or more room devices positioned within room 58.

Communication outlet 64 is electrically coupled to one or more cables, wires, or other conductors 68 that electrically couple the communication outlet 64 to a nurse call system 70 and one or more conventional room devices, such as a television 72, a room light 74, and/or a reading light 76. Conductors 68 are typically located behind wall 62 and not visible. In some healthcare facilities, conductors 68 may first couple to a room interface circuit board that includes one or more conductors 68 for electrically coupling the room interface circuit board to room device 72, 74, 76 and/or nurse call system 70. Still other communicative arrangements for coupling communication outlet 64 to nurse call system 70 and/or one or more room devices 72, 74, 76 are possible.

Nurse call cable 66 (FIG. 4) enables linked locator unit 60 to communicate with nurse call system 70 and/or room devices 72, 74, 76. Because patient support apparatus 20 is able to wirelessly communicate with linked locator unit 60, patient support apparatus 20 is thereby able to communicate with nurse call system 70 and room devices 72, 74, 76. A patient supported on patient support apparatus 20 who activates a nurse call control (e.g. 50g; see FIG. 3) on patient support apparatus 20 causes a signal to be wirelessly sent from patient support apparatus 20 to linked locator unit 60, which in turn conveys the signal via nurse call cable 66 to the nurse call system 70, which forwards the signal to one or more remotely located nurses (e.g. nurses at one or more nurse's stations 78). If the patient activates one or more room device controls (e.g. controls 501-t; see FIG. 3), one or more wireless signals are conveyed to linked locator unit 60, which in turn sends appropriate signals via nurse call cable 66 to communication outlet 64 and the room device 72, 74, 76 that change one or more features of these devices (e.g. the volume, channel, on/off state, etc.).

As is also shown in FIG. 4, patient support apparatus 20 is further configured to communicate with a local area network 80 of the healthcare facility. In the embodiment shown in FIG. 4, patient support apparatus 20 includes a wireless network transceiver 96 (FIG. 5) that communicates wirelessly with local area network 80. Network transceiver 96 is, in at least some embodiments, a WiFi transceiver (e.g. IEEE 802.11) that wirelessly communicates with one or more conventional wireless access points 82 of local area network 80. In other embodiments, network transceiver 96 may be a wireless transceiver that uses conventional 5G technology to communicate with network 80, one or more servers hosted thereon, and/or other devices. In some embodiments, network transceiver 96 may include any of the structures and/or functionality of the communication modules 56 disclosed in commonly assigned U.S. Pat. No. 10,500,401 issued to Michael Hayes and entitled NETWORK COMMUNICATION FOR PATIENT SUPPORT APPARATUSES, the complete disclosure of which is incorporated herein by reference. Still other types of wireless network transceivers may be utilized.

In some embodiments, network transceiver 96 is a wired transceiver that is adapted to allow patient support apparatus 20 to communicate with network 80 via a wired connection, such as an Ethernet cable that plugs into an Ethernet port (e.g. an RJ-45 style port, an 8P8C port, etc.) built into patient support apparatus 20. In still other embodiments, patient support apparatus 20 includes both a wired transceiver 96 for communicating with network 80 via a wired connection and a wireless transceiver 96 for wirelessly communicating with network 80.

Patient support apparatus 20 is configured to communicate with one or more servers on local area network 80 of the healthcare facility. One such server is a patient support apparatus server 84. Patient support apparatus server 84 is adapted, in at least one embodiment, to receive data from the patient support apparatuses 20 positioned within the healthcare facility and distribute this data to caregivers, other servers, and/or other software applications. As will be discussed in greater detail below, server 84 may also be configured to receive data from one or more devices 100 or and/or badges 142 that are positioned within one or more volumes of space defined around patient support apparatus 20 and/or within a volume of space defined around locator units 60. Such information may be forwarded from the device(s) to patient support apparatus 20, and from patient support apparatus 20 to server 84 via network transceiver 96 and one or more access points 82. In some embodiments where data from medical devices is collected, the data from one or more of the medical devices may be forwarded to an Electronic Medical Records (EMR) server 92, and/or to one or more other servers on network 80 (and/or one or more electronic devices 98), such as a caregiver assistance server and/or a caregiver assistance software application, as will also be discussed in greater detail below.

In some embodiments, patient support apparatus server 84 is configured to communicate at least some of the patient support apparatus data and/or device data received from patient support apparatuses 20 to a remote server 86 that is positioned geographically remotely from the healthcare facility. Such communication may take place via a conventional network appliance 88, such as, but not limited to, a router and/or a gateway, that is coupled to the Internet 90. The remote server 86, in turn, is also coupled to the Internet 90, and patient support apparatus server 84 is provided with the URL and/or other information necessary to communicate with remote server 86 via the Internet connection between network 80 and server 86.

In some alternative embodiments, patient support apparatus 20 may be configured to communicate directly with one or more cloud-based servers, such as remote server 86, without utilizing patient support apparatus server 84. That is, in some embodiments, patient support apparatuses 20 may be configured to communicate directly with a remote server without relying upon any locally hosted servers (e.g. servers hosted on network 80). Patient support apparatus 20 is provided with the URL and/or other information necessary to communicate with remote server 86 via the Internet connection between network 80 and remote server 86. In one embodiment, patient support apparatus 20 utilizes Microsoft's Azure could computing service to directly connect to one or more remote servers 86 without utilizing server 84. In some such embodiments, network appliance 88 is a router configured to support such direct connections. Still other types of direct-to-cloud connections may be utilized with one or more of patient support apparatuses 20. When patient support apparatus 20 is configured to directly communicate with remote server 86, patient support apparatus server 84 may be omitted and any one or more of the functions of patient support apparatus server 84 described herein may be performed by remote server 86.

Patient support apparatus server 84 is also configured to determine the location of each patient support apparatus 20 (and/or its associated devices), or receive the location of each patient support apparatus 20 (and/or its associated devices) from the patient support apparatuses 20 themselves. In some embodiments, patient support apparatus server 84 determines the room number and/or bay area of each patient support apparatus 20 and its associated devices that are positioned within a room 58, as well as the location of patient support apparatuses 20 and their associated devices that are positioned outside of a room 58, such as those that may be positioned in a hallway, a maintenance area, or some other area. In general, patient support apparatus server 84 may be configured to determine the position of any patient support apparatus 20 that is positioned within communication range of one or more locator units 60, as well as the location of any associated devices that are positioned within one or more volumes of space defined around the patient support apparatus 20, as will be discussed in greater detail below.

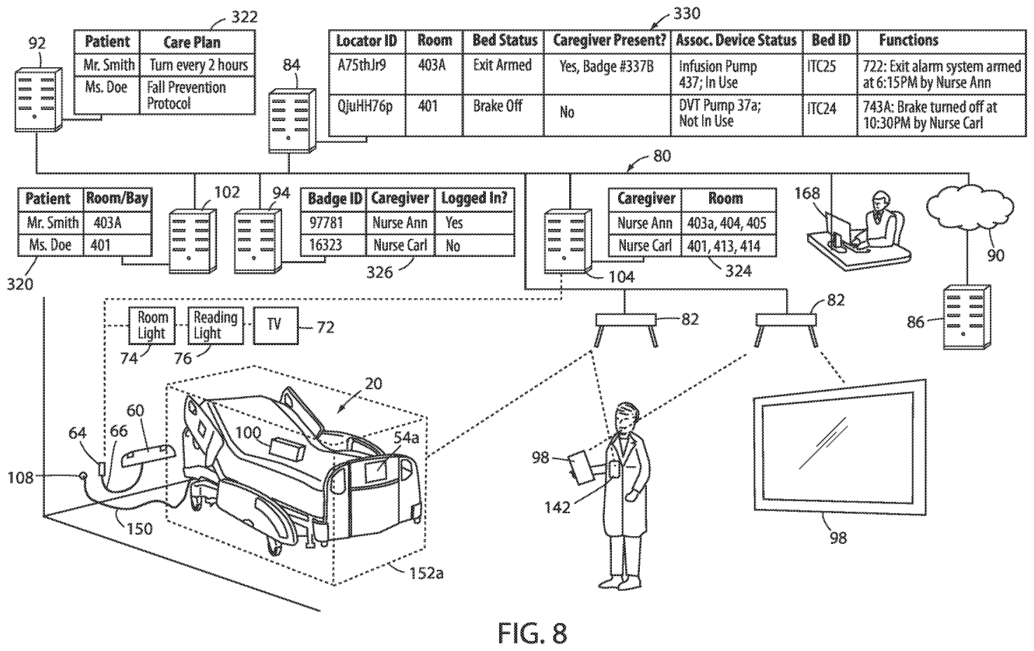

Patient support apparatus server 84 (FIGS. 4 & 8) is adapted to communicate with a plurality of other servers, such as a conventional EMR server 92, a conventional badge server 94, a conventional Admission, Discharge, and Transfer (ADT) server 102, and/or a conventional caregiver assignment server 104. EMR server 92 stores individual patient records. Such patient records identify a patient by name and include medical information associated with that patient. Such medical information may include all of the medical information generated from the patient's current stay in the healthcare facility as well as medical information from previous visits. EMR server 92 stores data such as that shown in table 322 (FIG. 8). Table 322 shows an abbreviated example of several types of medical information entries that are commonly found within a patient's medical records: a fall risk entry indicating whether the patient is a fall risk, a bed sore risk entry indicating whether the patient is at risk for developing bed sores, and a care plan for a particular patient. EMR server 92 includes far more additional information in the medical records of each patient than what is shown in table 322. It will be understood that the term “EMR server,” as used herein, also includes Electronic Health Records servers, or EHR servers for short, and that the present disclosure does not distinguish between electronic medical records and electronic health records.

Caregiver assignment server 104 (FIGS. 4 and 8) includes a caregiver assignment table 324 that matches caregivers to specific rooms and/or bays within the healthcare facility. Caregiver assignment server 104 stores information regarding shift changes, personnel, and the general assignments of caregivers who are employed by the healthcare facility. Although table 324 only shows two caregivers who are each assigned to three rooms, it will be understood that more than two caregivers will typically be present in a healthcare facility and they may have lessor or greater numbers of room assignments. In some caregiver assignment servers 104, caregivers are assigned to specific patients, rather than to specific rooms, in which case table 324 may correlate caregivers to individual patients rather than rooms. Still further, some conventional nurse call systems may be configured to carry out the functions of caregiver assignment server 104, in which case caregiver assignment server 104 may be replaced by and/or supplemented with a nurse call server. FIG. 8 shows an example of this where caregiver assignment server 104 is coupled by a dotted line to communication outlet 64. In this particular example, caregiver assignment server 104 also functions as a nurse call server that oversees the communications between patients and their caregivers.

ADT server 102 stores patient information, including the identity of patients and the corresponding rooms 58 and/or bays within rooms to which the patients are assigned. That is, ADT server 102 includes a patient-room assignment table 320 (FIG. 8). The patient-room assignment table 320 correlates rooms, as well as bays within multi-patient rooms, to the names of individual patients within the healthcare facility. The patient's names are entered into the ADT server 102 by one or more healthcare workers whenever a patient checks into the healthcare facility and the patient is assigned to a particular room within the healthcare facility. If and/or when a patient is transferred to a different room and/or discharged from the healthcare facility, the staff of the healthcare facility update ADT server 102. ADT server 102 therefore maintains an up-to-date table 320 that correlates patient names with their assigned rooms and/or bays. In some conventional electronic medical record systems, the functions of the ADT server 102 may be incorporated into the EMR system, and EMR server 92 may therefore, in some embodiments, carry out the functions of ADT server 102.

Badge server 94 (FIGS. 4 & 8) is configured to manage communications between, and keep track of the location of, badges 142 that are typically worn by healthcare workers, such as caregivers, service technicians, cleaning personnel, transportation assistants, etc. Badge server 94 typically maintains a table 326 (FIG. 8) that correlates badge IDs with individual healthcare workers. Each badge 142 includes a unique ID 166 (FIG. 5) that distinguishes that badge 142 from other badges 142. When a healthcare provider arrives at a healthcare facility, he or she typically grabs a badge 142 from a common collection of badges 142 and wears it for the duration of his or her work shift (and returns it to the general collection after his/her shift). In conventional badge servers 94, in order for server 94 to know that a particular badge 142 is assigned to a particular healthcare worker, the worker has to manually associate the particular badge 142 they chose to wear that day with their name (or some other worker ID). This manual association may involve scanning the badge on a badge reader in communication with server 94, typing information (such as the badge ID and/or the worker's ID) into a computer coupled to server 94, entering information into badge 142 (if it is a badge that allows data entry), performing other actions, and/or a combination of these steps.

Badge server 94 may also be configured to monitor the location of badges 142 within a healthcare facility. Typically this location monitoring is performed through the monitoring of the wireless access points 82 throughout the healthcare facility. That is, badges 142 are often equipped to use WiFi, or other wireless communication protocols, that allow them to communicate with wireless access points 82. By monitoring which access points the badges 142 are currently connected to, using a map of the location of the access point 82 within the facility, and, in some cases, using the signal strengths between the badges 142 and one or more of the access points 82, the general position of the badges 142 within the healthcare facility can be determined by badge server 94. Often this location determination is low resolution. That is, for example, this location-determining technique may only indicate which room the caregiver's badge 142 is currently located in, or which hallway, but might not provide accurate information as to where the badge 142 is located within a particular room or hallway. As will be discussed in greater detail below, patient support apparatuses 20 are equipped with a separate and independent location-determination technology that can be used to monitor the location of badges 142 (and/or other devices) with greater accuracy, such as locations that are accurate to within five to ten centimeters (although other accuracy levels may be utilized in accordance with this disclosure).

Badges 142 may be badges of the type sold or marketed by Stryker Corporation of Kalamazoo, Michigan, under the names Vocera Badge, Vocera Smartbadge, and/or Vocera Minibadge. Other types of badges may also, or alternatively, be used. Such badges 142 include the ability to transmit voice communications of healthcare workers to other badges 142 and/or other locations within a healthcare facility. Some of the badges may also include text messaging abilities, alarm notifications, and other functions. When integrated into the system described herein, such badges 142 may be modified to include one or more ultra-wideband transceivers and/or tags that communicate with ultra-wideband transceivers onboard patient support apparatus 20, as will be discussed in greater detail herein. That is, patient support apparatus 20 may be configured to repetitively determine the location of any of the badges 142 that are positioned within range of its ultra-wideband transceivers and determine whether the badge 142 is positioned inside or outside of one or more volumes of space, as will also be discussed in greater detail below. In some embodiments, badges 142 that do not include UWB transceivers may also be incorporated into the communication system described herein

In some embodiments, patient support apparatus server 84 (FIGS. 4 & 8) communicates with EMR server 92 in order to transmit patient data that is to be recorded in a patient's health record (e.g. vital sign readings from one or more vital sign sensors; weight readings taken from the scales built into patient support apparatuses 20; therapies provided to patients using a powered mattress 42 onboard patient support apparatuses 20; data from other devices that are determined to be associated with the patient assigned to patient support apparatus 20, etc.). In addition, server 84 communicates with EMR server 92, in some embodiments, in order to receive data from one or more of the devices that are being used with a particular patient.

It will be understood that the architecture and content of local area network 80 will vary from healthcare facility to healthcare facility, and that the examples shown in FIGS. 4 & 8 are merely two examples of the type of network a healthcare facility may be employ. Typically, one or more additional servers will be hosted on network 80 and one or more of them may be adapted to communicate with patient support apparatus server 84. Local area network 80 will also typically allow one or more electronic devices 98 to access the local area network 80 and the servers hosted thereon via wireless access points 82. Such electronic devices 98 include, but are not limited to, smart phones, tablet computers, portable laptops, desktop computers, smart televisions, and other types of electronic devices that include a WiFi capability and that are provided with the proper credentials (e.g. SSID, password, etc.) to access network 80 (and, in at least some situations, patient support apparatus server 84). As will be discussed in further detail herein, patient support apparatus server 84 is configured, in some embodiments, to share data with one or more electronic devices 98 that relates to patient support apparatus 20, that relates to one or more devices 100 that become associated with patient support apparatus 20 (or the patient assigned thereto), that relates to one or more badges 142 that become associated with patient support apparatus 20, and/or that relates to one or more medical records of the patient stored in EMR server 92.

Linked locator units 60 are adapted to wirelessly receive signals from patient support apparatus 20 and deliver the signals to communications outlet 64 in a manner that matches the way the signals would otherwise be delivered to communications outlet 64 if a conventional nurse call cable 66 were connected directly between patient support apparatus 20 (via a cable port 148; FIG. 5) and communications outlet 64. Linked locator units 60 are also adapted to transmit signals received from communications outlet 64 to patient support apparatus 20 via a BT transceiver 106 and/or a UWB transceiver 126 (FIG. 5). Thus, patient support apparatus 20 and linked locator unit 60 cooperate to send signals to, and receive signals from, communications outlet 64 in a manner that is transparent to communications outlet 64 such that outlet 64 cannot detect whether it is in communication with patient support apparatus 20 via a wired connection or it is in communication with patient support apparatus 20 via a wireless connection between patient support apparatus 20 and linked locator unit 60 (the latter of which is in wired communication with outlet 64). In this manner, a healthcare facility can utilize the wireless communication abilities of one or more patient support apparatuses 20 without having to make any changes to their existing communication outlets 64.

As noted, in addition to sending signals received from patient support apparatus 20 to communications outlet 64, linked locator units 60 are also adapted to forward signals received from communications outlet 64 to patient support apparatus 20. Linked locator units 60 are therefore adapted to provide bidirectional communication between patient support apparatus 20 and communications outlet 64. This bidirectional communication includes, but is not limited to, communicating command signals from any of controls 50 and/or from any of electronic devices 98 to corresponding room devices 72, 74, and/or 76 and communicating audio signals between a person supported on patient support apparatus 20 and a caregiver positioned remotely from patient support apparatus 20. The audio signals received by linked locator unit 60 from a microphone on patient support apparatus 20 are forwarded to communications outlet 64 (for forwarding to nurse call system 70), and the audio signals of a remotely positioned nurse that are received at communications outlet 64 (from nurse call system 70) are forwarded to a speaker onboard patient support apparatus 20.

Nurse call cable 66, in some embodiments, includes a conventional 37 pin connector on each end, one of which is adapted to be inserted into outlet 64 and the other one of which is adapted to be inserted into a linked locator unit 60 (or cable port 148 of patient support apparatus 20 if wired communication is desired). Such 37 pin connections are one of the most common types of connectors found on existing walls of medical facilities for making connections to the nurse call system 70 and room devices 72, 74, and 76. Linked locator unit 60 and nurse call cable 66 are therefore configured to mate with one of the most common type of communication outlets 64 used in medical facilities. Such 37 pin connectors, however, are not the only type of connectors, and it will be understood that linked locator units 60 can utilize different types of connectors that are adapted to electrically couple to different types of nurse call cables 66 and/or different types of communication outlets 64. One example of such an alternative communications outlet 64 and cable 66 is disclosed in commonly assigned U.S. patent application Ser. No. 14/819,844 filed Aug. 6, 2015 by inventors Krishna Bhimavarapu et al. and entitled PATIENT SUPPORT APPARATUSES WITH WIRELESS HEADWALL COMMUNICATION, the complete disclosure of which is incorporated herein by reference. Still other types of communication outlets 64 and corresponding connectors may be utilized.

Locator unit 60 (FIG. 4) also includes an electrical cord 150 having a plug positioned at a far end that is adapted to be inserted into a conventional electrical outlet 108. Electrical cord 150 enables locator unit 60 to receive power from the mains electrical supply via outlet 108. It will be appreciated that, in some embodiments, locator unit 60 is battery operated and cord 150 may be omitted. In still other embodiments, locator unit 60 may be both battery operated and include cord 150 so that in the event of a power failure, battery power supplies power to locator unit 60, and/or in the event of a battery failure, electrical power is received through outlet 108.

In some embodiments, locator units 60 include a video port 120 that is adapted to receive a display cable 110 (FIG. 4). The display cable 110 is adapted to couple to locator unit 60 at one end and a display device 56 at its opposite send. Locator unit 60 may be configured to use cable 110 to send data to display device 56 that is to be displayed thereon. Such data may include data from one or more devices 100 that are associated with the patient on patient support apparatus 20 (or with patient support apparatus 20 itself), data from one or more badges 142, data from one or more sensors onboard patient support apparatus 20, location data regarding the location of patient support apparatus 20, and/or other data. Cable 110 may be a High-Definition Multimedia Interface (HDMI) cable, a Video Graphics Array (VGA) cable, a DisplayPort (DP) cable, a plurality of Radio Corporation of America (RCA) cables, a Digital Visual Interface (DVI) cable, and/or another type of cable. Locator unit 60 is configured to include a complementary type of connector that mates with a connector on an end of cable 110. Further details regarding the communication between patient support apparatus 20 and display device 56 are provided below and, in some embodiments, patient support apparatus 20 may be configured to communicate directly with certain display devices without using locator unit 60 as a communication intermediary.

In addition to the other functions described herein, locator units 60 are configured to communicate location data to patient support apparatus 20 that enables patient support apparatus 20 and/or patient support apparatus server 84 to determine the location of patient support apparatus 20 within the healthcare facility. In general, such location determination is carried out by patient support apparatus 20 analyzing wireless signals communicated between itself and locator unit 60 in order to determine its position relative to locator unit 60. If patient support apparatus 20, or a predefined reference point on patient support apparatus 20 (e.g. its head end, its center, etc.) is positioned within a threshold distance of locator unit 60, patient support apparatus 20 associates itself with the locator unit 60. When associated, patient support apparatus 20 may communicate data to locator unit 60, receive data from locator unit 60, and also deem its location within the healthcare facility to be the same as location of locator unit 60. When patient support apparatus 20 is outside of the threshold distance, it does not associate itself with locator unit 60, and therefore does not exchange data with locator unit 60 or consider its location to be the same as that of locator unit 60's location.

In some embodiments, patient support apparatus 20 is configured to associate itself with a particular locator unit 60 if controller 140 determines that the locator unit 60 is within a volume of space 152a (FIGS. 4 & 6), or locator unit 60 determines that patient support apparatus 20 (or a reference point thereon) is positioned within volume of space 152a. In some embodiments, the volume of space 152a is defined with respect to each locator unit 60 and does not move. In other embodiments, the volume of space 152a is defined with respect to patient support apparatus 20 and moves as patient support apparatus 20 moves. In some embodiments, patient support apparatus 20 associates itself with a nearby locator unit 60 if both the locator unit 60 and the patient support apparatus 20 (or a reference point thereon) are concurrently within the predefined volume of space 152a. Regardless of whether volume of space 152a is defined with respect to a locator unit 60, or with respect to a patient support apparatus 20, by at least one or both of these devices (locator unit 60 and patient support apparatus 20) being positioned within the predefined volume of space 152a, the locator unit 60 and patient support apparatus 20 will be positioned within a threshold distance of each other. An example of patient support apparatus 20 occupying a volume of space 152a is shown in FIG. 4, where head end 38 of patient support apparatus 20 is positioned inside of volume of space 152a, and patient support apparatus 20 has therefore associated itself with that particular locator unit 60.

After associating itself with a particular locator unit 60, patient support apparatus 20 is configured to be able to have its absolute position within the healthcare facility determined by receiving a unique locator identifier (ID) 122 (FIG. 5) from the locator unit 60. The location of each locator unit 60 in the healthcare facility is surveyed during the installation of locator units 60, and the unique IDs 122 of each locator unit 60 are also recorded during the installation of locator units 60. This surveying information and corresponding ID information may be stored in patient support apparatus server 84 and/or onboard the patient support apparatuses 20, thereby enabling a patient support apparatus 20 and/or patient support apparatus server 84 to determine the location of a patient support apparatus 20 once it is associated with a particular locator unit 60.

In those embodiments where patient support apparatus server 84 is configured to determine the location of patient support apparatus 20, patient support apparatus 20 sends its relative position information with respect to the associated locator unit 60, and/or the ID 122 of the associated locator unit 60 (and its own unique patient support apparatus ID 130 (FIG. 5) to server 84. Server 84 includes a table of all of the locations of the locator units 60 (which, as noted, is generated via a surveying operation during the installation of locator units 60), and it uses that table to correlate the patient support apparatus IDs 130 and the locator unit IDs 122 it receives to specific locations within the healthcare facility. Thus, if a particular patient support apparatus 20 (with a particular ID 130) sends to server 84 an associated locator unit ID 122 that corresponds to room 430, server 84 determines that that particular patient support apparatus 20 is currently located in room 430. Generally speaking, and as will be discussed in greater detail below, the location of a patient support apparatus 20 is deemed to correspond to whichever locator unit 60 it is currently associated with, and if it is not currently associated with any locator unit 60, its location may be considered to be indeterminate.

In some embodiments of patient support apparatuses 20 and locator units 60, the relative location of a patient support apparatus 20 to a locator unit 60 is determined solely using ultra-wideband communication between the patient support apparatus 20 and the locator unit 60. Alternatively, in some embodiments, patient support apparatus 20 solely uses short range infrared communications with locator unit 60 to determine its relative location, wherein such short range infrared communications are only possible when the patient support apparatus 20 is positioned within a close proximity to the locator unit 60 (e.g. in the range of about 1-3 unobstructed meters). In these latter embodiments, patient support apparatus 20 may report that its location coincides with that of the nearby locator unit 60 when it is able to successfully communicate with the nearby locator unit 60 using these short range infrared communications. Still further, in some embodiments, patient support apparatus 20 and locator unit 60 may communicate with each other using both infrared and ultra-wideband communications. Further details regarding the use of short range infrared communications for location determination are described in commonly assigned U.S. Pat. No. 9,999,375 issued Jun. 19, 2018, to inventors Michael Hayes et al. and entitled LOCATION DETECTION SYSTEMS AND METHODS, the complete disclosure of which is incorporated herein by reference.

In some embodiments, locator units 60 and/or patient support apparatuses 20 may be constructed to include any or all of the functionality of the wireless headwall units and/or patient support apparatuses disclosed in commonly assigned U.S. patent application Ser. No. 14/819,844 filed Aug. 6, 2015, by inventors Krishna Bhimavarapu et al. and entitled PATIENT SUPPORT APPARATUSES WITH WIRELESS HEADWALL COMMUNICATION; in commonly assigned U.S. patent application Ser. No. 63/26,937 filed May 19, 2020, by inventors Alexander Bodurka et al. and entitled PATIENT SUPPORT APPARATUSES WITH HEADWALL COMMUNICATION; and/or in commonly assigned U.S. patent application Ser. No. 63/245,245 filed Sep. 17, 2021, by inventors Kirby Neihouser et al. and entitled SYSTEM FOR LOCATING PATIENT SUPPORT APPARATUSES, the complete disclosures of all of which are incorporated herein by reference.

Still further, in some embodiments, locator units 60 and/or patient support apparatuses 20 may be constructed to include any of the features and/or functions of the headwall units 144a and/or patient support apparatuses disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference.

FIG. 5 depicts a block diagram of patient support apparatus 20, a linked locator unit 60, a device 100, a badge 142, a display device 56, and network 80. As will be discussed in greater detail below, patient support apparatus 20 is configured to automatically determine the location of one or more locator units 60, badges 142, and/or other devices 100 that either have built-in UWB transceivers or a UWB tag attached to them. In addition, patient support apparatus 20 is configured to automatically carry out communications with these devices (60, 100, or 142) if they are positioned within a defined proximity to patient support apparatus 20. In some embodiments, if a particular device is positioned within the defined proximity, patient support apparatus 20 automatically associates the device with the patient assigned to patient support apparatus 20 (and/or with patient support apparatus 20 itself), and causes data from that device (or devices) to be displayed on one or more of display devices 56, and/or forwards data from that device (or devices) to patient support apparatus server 84 and/or EMR server 92 in communication with network 80. When the device is positioned outside the defined proximity, patient support apparatus 20 may automatically disassociate itself from the device and, in some situations, terminate communications with the device and/or inform patient support apparatus server 84 of the disassociation.

Linked locator unit 60 (FIG. 5) includes an ultra-wideband transceiver 126, a Bluetooth transceiver 106, a locator unit controller 112, configuration circuitry 114, a television controller 116, a headwall interface 118, a video port 120, a unit ID 122, and, in some embodiments, an infrared transceiver 124. Bluetooth transceiver 106 is adapted to communicate with a Bluetooth transceiver 128 onboard patient support apparatus 20 using RF waves in accordance with conventional Bluetooth standards (e.g. IEEE 802.14.1 and/or any of the standards maintained by the Bluetooth Special Interest Group (SIG) of Kirkland, Washington, USA). In some embodiments, transceivers 106 and 128 utilize Bluetooth Low Energy communications.

Ultra-wideband transceiver 126 is adapted to communicate with one or more ultra-wideband transceivers 132 positioned onboard patient support apparatus 20. Transceiver 126 is adapted to determine a distance between itself and patient support apparatus 20. Alternatively, or additionally, transceiver 126 may be adapted to allow one or more of the UWB transceivers 132 onboard patient support apparatus 20 to determine their distance(s) from transceiver 126. In some embodiments, transceivers 126 and 132 use time of flight (TOF) computations to determine these distances. In other embodiments, transceivers 126 and 132 may utilize other techniques (e.g. time difference of arrival, two-way ranging, angle of arrival, channel state information, etc.) for determining their distances from each other, either in addition to, or in lieu of, TOF computations. In some embodiments, transceivers 126, 132 may also determine an angle between themselves using angular information derived from antenna arrays positions onboard transceivers 126, 132, or by using other techniques. The position and orientation of each transceiver 132 onboard patient support apparatus 20 is known and stored in an onboard memory 134 and used to determine the position and orientation of patient support apparatus 20 with respect to the locator unit(s) 60 with which it is communicating. Such position and orientation information may be determined using conventional trilateration and/or triangulation techniques, or other techniques.

In some embodiments, transceivers 126, 132 are implemented as any of the Trimension™ ultra-wideband modules available from NXP Semiconductors of Austin, Texas. These modules include, but are not limited to, the Trimension™ UWB modules ASMOP1BO0N1, ASMOP1CO0R1, and/or the ASMOP1CO0A1, that utilize any of the following chips: the NXP SR150, SR100T, SR040, NCJ29D5, and/or the OL23DO chips. Modules manufactured and/or marketed by other companies may also be used, including, but not limited to, the Decawave DWM1000, DWM10001C, DWM3000 modules (available from Decawave of Dublin, Ireland); the Nordic TSG5162 SiP module (available from Tsingoal Technology of Beijing, China); and/or the UWB hub, wand, and/or sensors available from Zebra technologies of Lincolnshire, Illinois. Still other types of UWB modules may be used to implement transceivers 126 and 132.

Locator unit controller 112 is adapted to control the operation of transceivers 126, 106, configuration circuitry 114, TV controller 116, headwall interface 118, video port 120, and, if included, IR transceiver 124 (FIG. 5). When infrared transceiver 124 is included, it may be included to provide backwards compatibility to patient support apparatuses 20 that are not equipped with a UWB transceiver 132. That is, some healthcare facilities may include one or more patient support apparatuses that are not equipped with a UWB transceiver 132, but that do include an IR transceiver that is adapted to communicate with IR transceiver 124. When locator unit 60 includes IR transceiver 124, it is able to communicate its unit ID 122 to such patient support apparatuses via IR transceiver 124, which is a short range transceiver that is configured to only communicate with an adjacent patient support apparatus when the patient support apparatus is nearby (e.g. without about five feet or so). Such an adjacent patient support apparatus 20 then communicates the received locator unit ID 122 along with its own unique ID 130 (FIG. 5) to server 84 which, as noted previously, is able to correlate the locator unit ID 122 to a particular location with the healthcare facility. In this manner, server 84 is able to use locator units 60 determine the location of versions of patient support apparatuses 20 that don't have a UWB transceiver 132, but that do have an IR transceiver.

Headwall interface 118 is adapted to change the electrical state of one or more pins that are in electrical communication with communication outlet 64 (via cable 66). Headwall interface 118 changes these electrical states in response to instructions from controller 112. For example, if the exit detection system 136 of patient support apparatus 20 detects a patient exit, a controller 140 of patient support apparatus 20 sends an exit alert signal to linked locator unit 60 and controller 112 responds by instructing headwall interface 118 to change the electrical state of at least one pin that is used to signal an exit alert (or a generic priority alert) to the nurse call system 70 via communications outlet 64. Additionally, if a device 100, such as a portable exit detection sensor, is associated with patient support apparatus 20 and it detects a patient exit, the exit detection sensor may transmit an exit detection alert signal to patient support apparatus 20, which in turn forwards the exit alert signal to linked locator unit 60, and controller 112 responds by instructing headwall interface 118 to change the electrical state of the same pin or pins that it does in response to receiving an exit detection alert from exit detection system 136.

In some embodiments, headwall interface 118 may be constructed in the same manner as, and/or may include any one or of the functions as, the cable interface 88 described in commonly assigned U.S. patent application Ser. No. 63/193,778 filed May 27, 2021, by inventors Krishna Bhimavarapu et al. and entitled PATIENT SUPPORT APPARATUS AND HEADWALL UNIT SYNCING, the complete disclosure of which is incorporated herein by reference. Alternatively, or additionally, headwall interface 118 may be constructed in the same manner as, and/or may include any one or more of the same functions as, the headwall interface 120 disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference. Linked locator unit 60 may also be configured to perform any of the functions of the headwall units 94 disclosed in the above-mentioned '778 patent application.

Configuration circuitry 114 and TV controller 116 may be configured to perform any of the same functions as, and/or be constructed in any of the same manners as, the configuration circuitry 132 and the TV control circuit 134, respectively, of commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which has already been incorporated herein by reference. Additionally, or alternatively, linked locator unit 60 may be configured to perform any of the functions of the headwall units 144 disclosed in the aforementioned '508 patent application.

In the example shown in FIG. 5, patient support apparatus 20 includes a controller 140, a memory 134, exit detection system 136, a scale system 144, monitoring system 138, a microphone 146, Bluetooth transceiver 128, one or more UWB transceivers 132, display 52 (which may be part of control panel 54a, and/or another control panel 54), network transceiver 96, a nurse call interface 154, a propulsion system 212, a brake 214, and one or more actuators 216. Patient support apparatus 20 may also include a plurality of additional components that are not shown in FIG. 5. In other embodiments, patient support apparatus 20 may include different a different combination of some or all of the components shown in FIG. 5 and/or other components not shown therein. In some embodiments, patient support apparatus 20 may omit one or more of the components shown in FIG. 5.

Each UWB transceiver 132 is positioned at a known location on patient support apparatus 20. This known location information is stored in memory 134 and/or elsewhere, and may be defined with respect to any suitable frame of reference that is common to patient support apparatus 20. The known location information may include the spatial relationship between UWB transceivers 132 and/or any other components of patient support apparatus 20. For example, in some embodiments, the known location information includes the spatial relationship not only between UWB transceivers 132, but also the spatial relationships between UWB transceivers 132 and one or more of the following: the head end 38 of patient support apparatus 20, the foot end 40 of patient support apparatus 20, the sides of patient support apparatus 20, a reference point defined on patient support apparatus 20, the floor, and/or other components and/or landmarks of patient support apparatus 20. In some embodiments, this location information is used to determine the orientation of patient support apparatus 20 with respect to one or more walls 62, locator units 60, another patient support apparatus 20, and/or another object or structure within the healthcare facility.

In some embodiments, patient support apparatus 20 includes four UWB transceivers 132, each of which are positioned generally adjacent one of the four corners of patient support apparatus 20. In some such embodiments, the four UWB transceiver 132 are attached to, or positioned near, the four corners of litter frame 28. In other embodiments, the four UWB transceivers 132 are attached to, or positioned near, the four corners of base 22. In some embodiments, each of the four UWB transceivers 132 are attached to the corners of support deck 30. Still other locations of the UWB transceivers 132, as well as different numbers of the UWB transceiver 132, may be incorporated into patient support apparatus 20. In those embodiments of patient support apparatus 20 where one or more of the UWB transceivers 132 are coupled to components of patient support apparatus 20 that are movable (e.g. litter frame 28, which can have its height and orientation changed; or support deck 30 that can have its sections, such as head section 44, pivoted), sensors are included within patient support apparatus 20 that communicate the current position of the movable component to controller 140 so that controller 140 is able to determine the current positions of the UWB transceivers 132 and use those positions when determining the current location of a device, such as a badge 142 and/or another device 100.

Controller 140, as well as controller 112, may take on a variety of different forms. In the illustrated embodiment, each of these controllers is implemented as a conventional microcontroller. However, these controllers may be modified to use a variety of other types of circuits—either alone or in combination with one or more microcontrollers—such as, but not limited to, any one or more microprocessors, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. The instructions followed by controllers 112 and 140 when carrying out the functions described herein, as well as the data necessary for carrying out these functions, are stored in a corresponding memory that is accessible to that particular controller (e.g. memory 134 for controller 140, and a memory (not shown) for controller 112). In some embodiments, controller 140 may include and/or work with a microcontroller that is integrated into, or associated with, UWB transceiver(s) 132, and controller 112 may include and/or work with a microcontroller that is integrated into, or associated with, UWB transceiver 126.

Controller 140 (FIG. 5) utilizes UWB transceivers 132 to determine the relative position of patient support apparatus 20 with respect to one or more nearby locator units 60, one or more devices 100, and/or one or more badges 142. If patient support apparatus 20 is positioned within range of a locator unit 60, its UWB transceivers 132 communicate with the UWB transceiver 126 positioned on that locator unit 60, and the transceivers 132 and 126 exchange signals that enable them to determine the distances between themselves. This distance determination is done for each UWB transceiver 132 positioned onboard patient support apparatus 20 (or for as many as is necessary in order to determine an accurate position of locator unit 60 relative to patient support apparatus 20).

In some embodiments, UWB transceivers 126, 132 may also be configured to determine an angular relationships between themselves. The distance (and angle information) in at least some embodiments is calculated by UWB transceiver 132 and controller 140 of patient support apparatus 20. In other embodiments, UWB transceiver 126 and controller 112 may calculate the distance (and angle information) and forward the results of this calculation to patient support apparatus 20 (either via UWB transceiver 126 or BT transceiver 106). In either situation, patient support apparatus controller 140 is informed of the distances (and, in some embodiments, as noted, the angle information) between transceivers 132 and 126. These distances and orientations are then used to calculate a relative position of patient support apparatus 20 to the locator unit 60 in a common frame of reference that may be defined in a fixed relationship to the patient support apparatus 20 or in a fixed relationship to the locator unit 60.

Although FIGS. 4 and 5 only illustrate a single locator unit 60, it will be understood that a typical healthcare facility will include multiple locator units 60 positioned at different locations throughout the facility, including ones positioned within patient rooms and others positioned outside of patient rooms. Typically, at least one linked locator unit 60 will be positioned in each patient room of the healthcare facility, and if the patient room is intended to be occupied by more than one patient (e.g. it includes multiple bays), then additional linked locator units 60 may be included so that each patient support apparatus 20 will have a linked locator unit 60 positioned adjacent to each bay area in the room. Additional locator units 60, such as unlinked locator units 60a, may also be positioned at other locations through the healthcare facility.

The location of patient support apparatus 20 relative to locator units 60 is repetitively determined by an exchange of signals between UWB transceivers 126 and 132. This exchange is initiated by an interrogation signal that may be sent by the UWB transceivers 126 of the locator unit 60, and/or it may be sent by the UWB transceivers 132 of the patient support apparatuses 20. The trigger for sending these interrogation signals (from either source) may simply be the passage of a predefined interval of time, in at least some embodiments. That is, in some embodiments, patient support apparatus 20 and/or locator units 60 may be configured to periodically send out an interrogation signal that will be responded to by any UWB transceivers 126 or 132 that are positioned with range of that signal. In those embodiments where patient support apparatuses 20 are configured to send out such an interrogation signal, the time intervals between the interrogation signals may be varied depending upon the location, the number of devices 100 and/or badges 142 (if any) that are positioned within range of patient support apparatus 20, and/or the status of the patient support apparatus 20. For example, in some embodiments, controller 140 may be configured to send out the interrogation signals with longer timer intervals between them when the patient support apparatus is stationary (and, in some cases, when no devices 100 or badges 142 are currently positioned in communication range), and to send out the interrogation signals with shorter time intervals between them when the patient support apparatus 20 is in motion and/or when at least one device 100 or badge 142 is currently positioned within communication range of transceivers 132. In any of the aforementioned embodiments, motion of the patient support apparatus 20 may be detected in any suitable manner, such as by including one or more motion sensors on the patient support apparatus 20 (e.g. one or more accelerometers), and/or by monitoring the values of the repetitive distance measurements and looking for changes indicative of movement.

The measured distances (and/or angular information between locator units 60 and patient support apparatuses 20) that are generated from the communications between UWB transceivers 126, 132 may utilize Angle of Arrival (AoA) information, Time of Flight (TOF) information, Channel State Information, Time Difference of Arrival (TDoA) information, Two-Way Ranging (TWR) ranging information, and/or other information. In some embodiments, each transceiver 126, 132 includes an array of antennas that are used to generate distance and/or angular information with respect to the transceivers 126, 132 in which it is in communication. Still further, in some embodiments, UWB transceivers 126, 132 include one or more of their own microcontrollers, and the location of UWB transceivers 126, 132 may be determined by these internal microcontrollers without utilizing controller 140 and/or 112. In other embodiments, controllers 112 and/or 140 may work in conjunction with the microcontrollers of transceivers 126, 132 to determine their relative locations to each other.

Patient support apparatus 20 of FIG. 5 also includes a propulsion system 212. Propulsion system 212 is adapted to drive one or more wheels 24 of patient support apparatus 20 so that the caregiver doesn't have to exert manual force, or very little manual force, to move patient support apparatus 20 from one location to another. In some embodiments, patient support apparatus 20 may include an additional wheel that is larger than the wheels 24 shown in FIG. 1, and the propulsion system 212 may be adapted to drive that additional wheel. Further details of one example of the type of propulsion system that may be incorporated into patient support apparatus 20 are described in commonly assigned U.S. Pat. No. 10,603,234 issued Mar. 31, 2020, to inventors Thomas Puvogel et al. and entitled PATIENT SUPPORT APPARATUSES WITH DRIVE SYSTEMS, the complete disclosure of which is incorporated herein by reference. Other types of propulsion systems, of course, may be incorporated into patient support apparatus 20.

Patient support apparatus 20 also includes one or more actuators 216 that are adapted to move one or more components of patient support apparatus 20. Actuators 216 include lifts 26, an actuator used to pivot head section 44, and an actuator used to lift and lower a gatch onboard patient support apparatus 20. The gatch generally refers to the area of support deck 30 that corresponds to the knee area of the patient, and may be generally defined near the junction of the thigh section 46 with the foot section 48. In some embodiments, patient support apparatus 20 may include additional, or fewer, actuators 216. Regardless of the precise number of actuators 216, the actuators 216 may be hydraulic actuators, electric actuators, or any other suitable device for moving one or more components of patient support apparatus 20.

Nurse call interface 154 of patient support apparatus 20 (FIG. 5) includes Bluetooth transceiver 128 and a cable port 148, in some embodiments. Nurse call interface 154 provides an interface for patient support apparatus 20 to communicate with outlet 64 of nurse call system 70. That is, nurse call interface 154 provides the means for patient support apparatus 20 to bidirectionally communicate with communication outlet 64. As has been discussed, in some situations, patient support apparatus 20 uses Bluetooth transceiver 128 to communicate with Bluetooth transceiver 106 of linked locator unit 60, and linked locator unit 60 forwards communications back and forth between outlet 64 and patient support apparatus 20. In other words, in some situations, linked locator unit 60 functions as a communications intermediary between nurse call interface 154 and outlet 64. Alternatively, a nurse call cable 66 may be coupled directly between patient support apparatus 20 and wall outlet 64, thereby avoiding the need to use linked locator unit 60 as a communication intermediary. In such situations, one end of a nurse call cable 66 is plugged into cable port 148 of patient support apparatus 20 and the other end of the cable 66 is plugged directly into outlet 64. Nurse call interface 154 thereby provides patient support apparatus 20 with the ability to communicate either wirelessly or via wired means with the outlet 64.

Patient support apparatus 20 also includes, in at least some embodiments, a microphone 146 (FIG. 5) that is used to detect the voice of the patient when the patient wants to speak to a remotely positioned nurse. The patient's voice is converted to audio signals by microphone 146 and controller 140 is adapted to forward these audio signals to an adjacent communications outlet 64 positioned in wall 62 (FIG. 4). When a cable 66 is coupled between cable port 148 of patient support apparatus 20 and outlet 64, controller 140 forwards these audio signals to outlet 64 via the cable 66. When no such cable 66 extends between patient support apparatus 20 and outlet 64, controller 140 wirelessly forwards these audio signals to the linked locator unit 60 that it is currently associated with (using transceiver 128, or in some embodiments, one of transceivers 132) and controller 112 of linked locator unit 60 forwards these audio signals to outlet 64. As was noted, outlet 64 is in electrical communication with a conventional nurse call system 70 that is adapted to route the audio signals to the correct nurse's station 78, and/or other location. In some embodiments, microphone 146 acts as both a microphone and a speaker. In other embodiments, a separate speaker may be included in order to communicate the voice signals received from the remotely positioned nurse. In some embodiments, the audio communication between patient support apparatus 20 and communications outlet 64 is carried out in any of the manners, and/or includes any of the structures, disclosed in commonly assigned U.S. patent application Ser. No. 16/847,753 filed Apr. 14, 2020, by inventors Alexander Bodurka et al. and entitled PATIENT SUPPORT APPARATUSES WITH NURSE CALL AUDIO MANAGEMENT, the complete disclosure of which is incorporated herein by reference.

After the installation of locator units 60 in a particular healthcare facility, the location of each locator unit 60 within that facility is recorded. In some embodiments, the coordinates of the locations of locator units 60 are recorded in a common frame of reference (or converted to a common frame of reference after recordation). Such coordinates may be three dimensional (i.e. include a vertical and two horizontal components), or they may be two dimensional (no height component). In other embodiments, a more generalized location of one or more locator units 60 is determined, rather than the precise coordinates of the locator units 60. The generalized location of the locator units 60 may include an indication of the room, bay, area, hallway, portion of a hallway, wing, maintenance area, etc. that the locator unit 60 is positioned in. In still other embodiments, the locations of one or more locator units 60 are determined both generally and more precisely.

Regardless of how the location of each locator unit 60 is initially determined after they are installed in a healthcare facility (e.g. whether their coordinates are determined or a more generalized location is determined), the locations of all of the locator units 60, as well as their unique IDs 122, are stored in a memory accessible to server 84. Server 84 then uses this location data and ID data to determine the location of a patient support apparatus 20 (as well as the location of associated devices 100 and badges 142). Alternatively, or additionally, the location data and IDs 122 are forwarded to patient support apparatuses 20 for storage in their onboard memories 134 and for use in determining their own locations. In some embodiments, the location of each locator unit 60 (whether specific and/or general) may also, or alternatively, be stored in a memory within that particular locator unit 60 and shared with the devices it communicates with (e.g. patient support apparatuses 20). In some other embodiments, the location of each locator unit 60 may be stored in multiple locations.

In some embodiments, patient support apparatus 20 and/or one or more unlinked locator unit 60a are configured to automatically determine the direction of movement of patient support apparatus 20 as patient support apparatus 20 passes by the unlinked locator unit 60a. In such embodiments, which will be described in greater detail below, such unlinked locator units 60a may be positioned near entrances (e.g. hallways, doorways, etc.) into one or more particular areas of a healthcare facility, such as a department, wing, floor, unit, or the like. In this manner, patient support apparatus server 84 is able to determine a generalized location of a patient support apparatus 20 in certain areas of the healthcare facility that may be lacking locator units 60, or that may have a low density of locator units 60. For example, if it is desirable for a healthcare facility to be able to know that a particular patient support apparatus 20 is located in the emergency department, but it is not necessary to know specifically where in the emergency department, unlinked locators units 60a may be positioned at all of the entrances (which are also exits when the patient support apparatus 20 is traveling in the opposite direction) to the emergency department so that, whenever a particular patient support apparatus 20 moves past one of these entrances (either into or out of the emergency department), it can be determined whether the patient support apparatus is leaving or entering the emergency department.

It will be appreciated that patient support apparatuses 20 are configured to communicate with locator units 60 regardless of the orientation of the patient support apparatus 20. That is, the UWB transceivers 126 and 132 are radio frequency transceivers that do not rely on line of sight communication, unlike the IR transceiver 124 (if present). Thus, the patient support apparatuses 20 do not have to be pointed in any particular direction with respect to the locator units in order for transceivers 126 and 132 to communicate. This differs from some prior art systems that use IR communication between the patient support apparatuses 20 and the locator units and that require the IR transceiver onboard the patient support apparatus to be aimed toward the locator unit in order for communication to be established. It will also be understood that locator units 60 can be positioned on walls, columns, ceilings, or any other fixed structures within the healthcare facility.

Patient support apparatus 20 is also configured to use UWB transceivers 132 to determine the position of other devices relative to patient support apparatus 20, such as one or more badges 142, one or more other devices 100, and/or one or more electronic devices 98 (FIG. 5). As will be discussed in greater detail below, controller 140 uses UWB transceivers 132 to repetitively determine the relative position of these devices by communicating with one or more respective UWB transceivers that are either built into those devices or attached to a tag that is affixed to those devices. Such UWB transceivers operate in the same manner as UWB transceivers 132 and/or UWB transceiver 126 of locator units 60. And, as will also be discussed in greater detail below, controller 140 of patient support apparatus 20 uses the relative position information to determine how it will interact with these devices, including whether to associate with these devices or not. When controller 140 associates patient support apparatus 20 with one or more of these devices, as will be discussed in greater detail below, controller 140 and/or server 84 may take one or more of the following actions: display data from these devices on display 52 and/or display device 56; send data from one or more of these devices to patient support apparatus server 84, EMR server 92, and/or badge sever 94 via network transceiver 96; retrieve data about one or more of these devices from patient support apparatus server 84, EMR server 92, and/or badge sever 94 via network transceiver 96; send one or more signals from these devices to communication outlet 64 (via a cable 66 or through linked locator unit 60); forward one or more signals from outlet 64 to one or more of these devices; retrieve data from EMR server 92 that was generated by these devices; retrieve data from these devices via another route that is independent from EMR server 92; and/or take other actions. In some embodiments, as will be discussed in greater detail below, controller 140 may simply record the time, location, and/or ID of any UWB devices (98, 100, 142) that it is able to communicate with at a given time, thereby enabling a log of events to be created.

In the particular embodiment shown in FIG. 5, badge 142 includes a controller 156, a UWB transceiver 158, a microphone 160, a speaker 162, a network transceiver 164, and a unique ID 166. UWB transceiver 158 is adapted to communicate with the UWB transceivers 132 positioned onboard patient support apparatus 20 so that the position of badge 142 relative to patient support apparatus 20 can be repetitively determined. UWB transceiver 158 may be the same as all of the other UWB transceivers discussed herein (e.g. UWB transceivers 132, 126, etc.). UWB transceiver 158 is further adapted to transmit the unique ID 166 of badge 142 to patient support apparatus 20 so that patient support apparatus 20 knows which specific badge 142 it is communicating with. It will be understood that the components of badge 142 shown in FIG. 5 are merely an illustrative example of a badge 142, and that different badges may be utilized with the communication system of the present disclosure that have fewer, greater, or different components than those shown in FIG. 5. Those badges 142 that include a UWB transceiver 158 and identifier 166—which may be integrated into the badge 142 or part of a tag that is attached to the badge—will be able to fully operate in accordance with the principles discussed herein. However, badges that do not include a UWB transceiver 158 can still be integrated with the communications of the patient support apparatuses 20 and/or patient support apparatus server 84.

Controller 156 of badge 142 is adapted to oversee the operation of badge 142, process the communications of UWB transceiver 158 with other UWB transceivers (e.g. transceivers 132), respond to the activation of controls on badge 142 (if any), and oversee the operation of microphone 160 and speaker 162. Badge 142 is designed to be worn by a healthcare facility worker, or attached to a structure (e.g. necklace, bracelet, clip, etc.) that is adapted to be worn by a healthcare facility worker. Badge 142 of FIG. 5 is adapted to allow a caregiver to speak into microphone 160 and have his or her voice transmitted to another badge 142, and/or a computing device in communication with badge server 94. Similarly, badge 142 of FIG. 5 is adapted to receive audio signals from other badges 142, or computing devices, and to route them to speaker 162 so that the person wearing badge 142 can hear those audio signals. In other words, badge 142 is adapted to allow its wearer to communicate with remotely positioned personnel who are either wearing another badge 142 or who are able to access badge server 94 and communicate audio signals to/from server 94 using a conventional computing device (e.g. a desktop computer with a headset having a microphone and speakers).

Badge 142 communicates audio signals by routing them through badge server 94 using network transceiver 164. Network transceiver 164, like network transceiver 96 of patient support apparatus 20, may be a WiFi transceiver (e.g. IEEE 802.11) that wirelessly communicates with one or more conventional wireless access points 82 of local area network 80. In other embodiments, network transceiver 164 may be a wireless transceiver that uses conventional 5G technology to communicate with network 80 and badge server 94. In still other embodiments, network transceiver 164 may be a cellular transceiver that is adapted to use one or more protocols of conventional cell phones to communicate with badge server 94.

Regardless of the specific technology of network transceiver 164, when a user of a badge 142 calls another caregiver, controller 156 sends the user's audio signals to badge server 94 and badge server 94 routes those audio signals to the appropriate recipient (which, as noted, may be another badge 142 or a computing device in communication with badge server 94). Similarly, the recipient's audio signals are routed to badge server 94 and badge server 94 then forwards those to badge 142. In this manner, two-way voice communication is established between a first badge 142 and another badge 142 or computing device.

Controller 140 of patient support apparatus 20 (FIG. 5) repetitively determines the position of badges 142, locator units 60, devices 100, and/or electronic devices 98 after they move within communication range of UWB transceivers 132. Controller 140 uses UWB transceivers 132, which communicate with the UWB transceivers 158 of each badge 142 (or the UWB transceivers of locator units 60, devices 98 and/or 100) to repetitively determine the relative position of the badge 142 (or unit 60 or device 98 or 100) with respect to patient support apparatus 20. Controller 140 compares this relative position to a space volume 152c (FIGS. 4 & 6) and, if the badge 142 (or other device 60, 98, or 100) is outside of space volume 152c, it automatically disassociates badge 142 (or other device 60, 98, or 100) from patient support apparatus 20. On the other hand, as long as the position of badge 142 (or other device 60, 98, or 100) remains inside of space volume 152c, controller 140 maintains the association of badge 142 (or device 60, 98, or 100) with patient support apparatus 20. As will be discussed in greater detail below, once a badge 142 (or device 60, 98, or 100) is associated by controller 140 with a patient support apparatus 20, controller 140 may take one or more automatic actions, several of which will now be discussed.

In some embodiments, after controller 140 associates a badge 142 with patient support apparatus 20, it is configured to automatically determine a class of user to which the badge 142 belongs, and/or to send the badge ID 166 to patient support apparatus server 84 for patient support apparatus server 84 to determine the corresponding user class. That is, the unique ID 166 of each badge 142 may contain information that indicates which class of healthcare worker the badge 142 belongs to. Alternatively, it may be necessary to determine the class of worker to which a badge 142 belongs by utilizing the identity of the caregiver who is currently using that badge 142. In such cases, patient support apparatus server 84 is configured to receive information from one or more other servers on network 80 that store data indicating which workers within that particular healthcare facility belong to which classes of (e.g. a worker-ID-to-worker-class database). Such servers may include, but are not limited to, caregiver assignment server 104 and/or another server. In addition to determining the class of a particular healthcare worker, patient support apparatus server 84 is configured to utilize data that correlates badge IDs 166 to specific healthcare workers (e.g. a worker-ID-to-badge-ID database). Such data will typically be stored in badge server 94. By combining the data that correlates healthcare worker identities with the badge identifiers 166, along with data from badge server 84 that correlates badge identifiers 166 with healthcare worker identities, patient support apparatus server 84 is able to determine which class of healthcare worker a given badge 142 corresponds to.

The different classes of workers who wear badges 142 may vary from healthcare facility to healthcare facility. In one embodiment, the different classes of users may correspond to nurses, nursing assistants, doctors, service technicians, cleaning personnel, and transportation assistants (the latter of which are responsible for transporting patients to different locations within the healthcare facility). Server 84 may also be customizable to allow authorized personnel of a healthcare facility to define their own classes, and in some instances, such personnel may further refine one or more of the aforementioned classes. For example, some healthcare facilities may assign classes to certain types of doctors and/or nurses, such as emergency room doctors and/or nurses, pediatric doctors and/or nurses, cardiac doctors and/or nurses, etc. Other classification systems can, of course be used. Also, as noted above, the classification of healthcare workers may be defined on another server within the healthcare facility and read by patient support apparatus server 84. Alternatively, the classes of healthcare workers may be entered directly into patient support apparatus server 84 by authorized personnel, such as by utilizing a conventional computing device (e.g. computer 168 (FIG. 8)) that has access to network 80 and patient support apparatus server 84.

Regardless of the specific classifications that are implemented in a healthcare facility, patient support apparatus server 84 and/or controller 140 are configured to determine which class each badge 142 belongs to using the badge ID 166 and the other information mentioned above (a worker-ID-to-badge-ID database and a worker-ID-to-worker-class database). After making this determination, in some embodiments, controller 140 and/or patient support apparatus server 84 are configured to make one or more changes to patient support apparatus 20 in response to that determination, and/or to automatically take one or more other actions.

For example, in some embodiments, controller 140 and/or patient support apparatus server 84 are configured to automatically, and repetitively, determine the distance between a badge 142 positioned within space volume 152c and an ultra-wideband tag worn by the patient. The ultra-wideband tag may be incorporated into a wristband worn by the patient, an item of clothing, a necklace, or worn by the patient in some other manner. The ultra-wideband tag operates in the same manner as the devices 100 described herein and may be considered to be one type of device 100 with which controller 140 is able to communicate in accordance with the principles discussed herein.

Controller 140 and/or patient support apparatus server 84 are configured to repetitively determine the distance between badge 142 and the patient-worn tag by repetitively determining the three-dimensional position of each of these devices with respect to patient support apparatus 20. After the position of both the badge 142 and tag is known, controller 140 and/or patient support apparatus server 84 use conventional geometric techniques to determine the distance between the two. In some embodiments, controller 140 and/or patient support apparatus server 84 determine a two-dimensional distance between the two devices in a plane that is generally parallel to the floor on which patient support apparatus 20 is positioned. That is, in some embodiments, controller 140 and/or patient support apparatus server 84 determine how far away the badge 142 and tag are horizontally from each other, and don't calculate a height difference between the two. In other embodiments, controller 140 and/or patient support apparatus server 84 may calculate a three-dimensional distance between badge 142 and the patient's tag.

In those embodiments where controller 140 is configured to calculate the distance between a tag worn by the patient and a badge 142 that is positioned within space volume 152c, controller 140 may be configured to send that distance calculation to patient support apparatus server 84 via network transceiver 96. In those embodiments where patient support apparatus server 84 is configured to calculate this distance, controller 140 may be configured to send the repetitively-determined positions of the badge 142 relative to patient support apparatus 20 and the repetitively-determined positions of the patient-worn tag to patient support apparatus server 84. Patient support apparatus server 84 then uses these positions to repetitively calculate the distance between the tag and the badge 142.

In some embodiments, patient support apparatus server 84 and/or controller 140 are configured to issue a distance alert if the distance exceeds a threshold. In some such embodiments, controller 140 and/or patient support apparatus server 84 are configured to issue the distance alert if the distance exceeds a threshold and one or more additional criteria are met. For example, in some embodiments, the distance alert is not issued any time the patient is currently on patient support apparatus 20, which controller may determine through the scale system 144 and/or the position of the patient-worn tag. In some embodiments, the distance alert is not issued if the patient is out of patient support apparatus 20 and the patient is not considered a fall risk, regardless of what the distance is between the patient-worn tag and the caregiver badge 142. Such fall risk information may be retrieved by patient support apparatus server 84 and/or controller 140 automatically from EMR server 92.

In other embodiments, the decision by controller 140 and/or patient support apparatus server 84 to issue a distance alert may also be dependent upon one or more of the following factors (in addition to the distance exceeding a threshold): the class of healthcare worker to which the badge 142 belongs (e.g. a distance alert may be sent if the worker is service technician, rather than a nurse, nurse's assistant, or doctor), the location of the caregiver-worn tag, the location of the badge 142, the health condition of the patient (e.g. the malady for which the patient is visiting the healthcare facility), the time of day, the fall risk of the patient, a mobility score of the patient, and/or other factors.

The purpose of the calculation of the distance between the caregiver and the patient (i.e. the distance between the badge 142 and the patient-worn tag) is to ensure that the caregiver is accompanying the patient when the patient is moving around their room 58 and/or when the patient is getting into, or out of, patient support apparatus 20. Thus, the distance threshold may be set at a distance that ensures that the caregiver remains in close enough proximity to the patient to assist the patient as he or she moves. This may be particularly useful in situations where the patient is at risk of falling, and the caregiver is to accompany the patient as he/she moves in order to prevent the patient from falling. In such situations, the distance threshold may be on the order of two to five feet, although other thresholds may, of course, be utilized. It will also be understood that the monitoring of the distance between the patient and caregiver may be useful for other purposes besides fall prevention, and that the size of the threshold may vary considerably, depending upon the specific purpose of the distance monitoring function.

In some embodiments, patient support apparatus server 84 and/or controller 140 are configured to record the distances between the patient and the caregiver whenever the caregiver visits the patient, and/or whenever the caregiver visits the patient and the patient exits, or is otherwise out of, patient support apparatus 20. The recording of these distances may then be later reviewed by a healthcare facility administrator to ensure that the healthcare protocols of that particular healthcare facility are being followed by the caregivers when they visit with their patient.

In some embodiments, in addition to, or in lieu of, determining the distance between the patient and the caregiver, controller 140 is configured to repetitively monitor the position of the patient (via a UWB tag worn by the patient) and periodically report the patient's current position to patient support apparatus server 84. Controller 140 may thus be configured to periodically send location messages to patient support apparatus server 84 indicating the current position of the patient. The location information contained in the location messages may be a three-dimensional position of the patient's tag relative to the patient support apparatus 20, and/or it may be a more generalized indication of the location of the patient's position, such as an indication of the patient being in or our of patient support apparatus 20, the patient being outside of space volume 152c (and/or the room 58), the patient being in an adjacent restroom, the patient being seated in another patient support apparatus 20 within the same room, such as a recliner, or other type of chair), or still other type of location information.

Another action that controller 140 and/or patient support apparatus server 84 may be configured to make in response to determining the location, and/or class of worker, of a badge 142 is to automatically pause any alert being issued by patient support apparatus 20 when the badge 142 moves within a predefined space around patient support apparatus 20, such as, for example, volume of space 152c. Thus, if patient support apparatus 20 is issuing an alert and controller 140 detects that a badge 142 of a healthcare worker has entered volume of space 152c, controller 140 may be configured to automatically pause that alert if the badge 142 is being worn by a healthcare worker of a particular class, such as a caregiver (as opposed to a service technician, cleaning person, etc.). Controller 140 may be configured to continue to pause the alert for as long as a healthcare worker of the requisite class (as determined from their badge 142) remains within a desired position (e.g. space volume 152c).

The position of the healthcare worker relative to patient support apparatus 20 that is necessary to cause controller 140 to pause the alert may vary for different types of alerts. For example, if patient support apparatus 20 is issuing an exit alert because exit detection system 136 (FIG. 5) has detected that the patient is in the process of exiting patient support apparatus 20, controller 140 may pause the exit detection alert when the healthcare worker is positioned somewhere around the perimeter of patient support apparatus 20 (where the healthcare worker would be able to assist the patient into, or out of, patient support apparatus 20). Alternatively, or additionally, in the specific case of an exit detection alert, controller 140 may be configured to pause the alert so long as the healthcare worker's badge 142 remained within the threshold distance discussed above with respect to a UWB tag worn by the patient. In this example, the exit alert would be paused for as long as the healthcare worker remained in close proximity to the patient, which would ensure that the healthcare worker was close enough to the patient to help prevent the patient from falling while the patient was out of patient support apparatus 20.

Another type of alert that controller 140 may issue, and automatically pause when a particular type of healthcare worker enters a defined vicinity of patient support apparatus 20, is an alert issued by monitoring system 138 (FIG. 5). As was noted, monitoring system 138, when armed, monitors a plurality of conditions of patient support apparatus 20 and issues an alert if any one or more of the conditions are in an undesired state. Such conditions may include, but are not limited to, a state of a siderail 36 on patient support apparatus 20; a state of a brake on the patient support apparatus 20; a height of the litter frame 28; a charge state of a battery onboard patient support apparatus 20; a connection strength of the network transceiver 96 to the healthcare facility computer network 80; an armed or disarmed state of exit detection system 136; a state of power cable 150 (e.g. plugged into power outlet 108 or not); a state of the wireless connection between patient support apparatus 20 and a locator unit 60; a state of cable 66 between a locator unit 60 and communication outlet 64 (e.g. plugged in or not); a state of cable 66 between patient support apparatus 20 and communication outlet 64 (e.g. plugged in or not); an un-zeroed state of scale system 144; a Head of Bed (HoB) angle of head section 44 with respect to horizontal; and/or other states.

Controller 140 may be configured to react to the nearby presence of a caregiver badge 142 with respect to an alert from monitoring system 138 in a manner different from how it reacts to the presence of a nearby caregiver badge 142 with respect to an alert from exit detection system 136. Or, in some embodiments, it may react in the same manner. In those embodiments where it may react in a different manner, controller 140 may be configured to use a larger volume of space as the trigger for pausing the alarm. In other words, controller 140 may be configured to pause the monitoring system 138 alert when the healthcare worker's badge 142 comes within a threshold distance of patient support apparatus 20 that is larger than the threshold distance used by controller 140 when pausing an alert from exit detection system 136. Thus, for example, a caregiver may only need to enter the room 58 in order to cause controller 140 to pause an alert from monitoring system 138, while a caregiver may need to not only come into the room, but also be positioned around the perimeter of patient support apparatus 20, before controller 140 pauses an alert from exit detection system. However, as noted, in some embodiments, controller 140 may be configured to react to the presence of a healthcare worker's badge 142 in the same manner for alerts generated by exit detection system 136 and by monitoring system 138.

In addition to pausing an alert when a caregiver's badge 142 is positioned within a predetermined space volume (e.g. 152b, 152c, and/or another volume), controller 140 is configured to unpause the alert when the caregiver's badge moves outside of the predetermined space volume without the underlying cause for the alert having been rectified. Therefore, for example, if monitoring system 138 issues an alert because a brake on patient support apparatus 20 is deactivated, controller 140 may automatically pause the alert when the caregiver enters the room 58, but then re-activate the alert if the caregiver leaves the room and the brake is still deactivated.

The manner in which controller 140 automatically pauses an alert in response to the nearby presence of a healthcare worker's badge 142 may take on different forms. If the alert is purely local (i.e. it involves only lights and/or sounds emitted from patient support apparatus 20, controller 140 stops the activation of the alerting lights and/or stops the emission of the alerting sound(s) from any sound-emitting devices onboard patient support apparatus 20. The sound emitting devices may include buzzers, speakers, and/or other types of sound emitting devices. If the caregiver leaves the vicinity of patient support apparatus 20 without rectifying the cause of the underlying alert, controller 140 reactivates the alerting lights and/or the sound-emitting device(s).

In some embodiments, controller 140 is configured to issue an alert by changing an electrical state of one or more pins in communication outlet 64. In such embodiments, controller 140 is configured to automatically pause such an alert by changing the electrical state of the one or more pins back to their non-alerting state, and to unpause the alert by returning the electrical state of the one or more pins back to their alerting state. This is accomplished either through changing the state of one or more relays onboard patient support apparatus 20 (if it is directly coupled by cable 66 to outlet 64), or by sending a message to an associated linked locator unit 60 (if the patient support apparatus 20 is using a linked locator unit 60 as a communications intermediary between itself and communication outlet 64). In the former case, the relays onboard patient support apparatus 20 are in electrical communication with the pins of the cable 66, and the changing of the relay states therefore causing a corresponding change in the electrical state of one or more pins in communications outlet 64. In the latter case, controller 112 of the linked locator unit 60 instructs headwall interface 118 to make changes to the electrical state of at least one of the pins that are coupled to communications outlet 64 by way of cable 66.

In some embodiments, controller 140 is configured to issue an alert by sending a message to patient support apparatus server 84, to badge server 94, and/or to another server. In such embodiments, controller 140 is configured to automatically pause such an alert by sending a second message to patient support apparatus server 84, to badge server 94, and/or to another server. The second message instructs the recipient server that the alert has been cancelled. The server then reacts in the manner is has been to configured to react to the cancellation of the alert. If the caregiver leaves the vicinity of patient support apparatus 20 without rectifying the cause of the underlying alert, controller 140 sends a third message to patient support apparatus server 84, to badge server 94, and/or to another server. The third message instructs the server that the alert has been re-issued.

In some embodiments, controller 140 is configured to issue an alert by sending a message to one or more badges 142. These alerts may be sent through badge server 94 and/or they may be sent directly to one or more badges 142 via Bluetooth transceiver 128 and/or via one or more of the UWB transceivers 132. In such embodiments, controller 140 is configured to automatically pause such an alert by sending a second message to the badges 142. The second message instructs the recipient badges 142 that the alert has been cancelled. The badge 142 then reacts in the manner is has been to configured to react to the cancellation of the alert. If the caregiver leaves the vicinity of patient support apparatus 20 without rectifying the cause of the underlying alert, controller 140 sends a third message to those same badges 142 that notifies the badges 142 that the alert has been re-issued.

Another action that controller 140 and/or patient support apparatus server 84 may be configured to automatically carry out is to confirm, and/or ensure, that appropriate personnel respond to certain specified events, such as, but not limited to patient emergencies. Such emergency events may include the patient going into cardiac or respiratory arrest, suffering seizures, falling down, the triggering of specific codes in the healthcare facility (e.g. code blue, code red, etc.), and/or other events. In some embodiments, patient support apparatus server 84 is customizable to allow authorized personnel of a healthcare facility to enter and/or specify the set of events that controller 140 and/or patient support apparatus server 84 will automatically respond to. Alternatively, or additionally, patient support apparatus server 84 may be configured to access another server or database to retrieve the healthcare facility's definition of specified events.

Patient support apparatus server 84 and/or controller 140 may be configured to detect the existence of a specified event in different manners. In one embodiment, patient support apparatus 20 includes a CPR release handle that, when pulled, automatically moves support deck 30 to a flat position (and may also change litter frame 28 to a specific height), and controller 140 may be configured to treat the pulling of the CPR release handle as an emergency event. Patient support apparatus server 84 and/or controller 140 may also be configured to access one or more other servers on network 80 to determine when a specified event exists. In some situations, the existence of a specified event is detected by a conventional nurse call server (which may also house the data of caregiver assignment server 104, as noted above). Healthcare facilities often include buttons or switches in the rooms 58 of the healthcare facilities that caregivers can activate when an emergency event occurs, and those switches or buttons are often connected to the existing nurse call system. In such situations, patient support apparatus server 84 is configured to retrieve data from the nurse call server to determine when an emergency event is occurring.

When patient support apparatus server 84 receives information indicating that a specified event is occurring, it consults a database (which may be internal, or stored elsewhere on network 80) that indicates what types of personnel the healthcare facility wants to have respond to that type of event. This database may be stored internally within patient support apparatus server 84 itself, or it may be stored in another server on network 80. In some embodiments, patient support apparatus server 84 is customizable to allow authorized personnel of a healthcare facility to enter and/or define the set of personnel who should respond to different types of events. Alternatively, or additionally, patient support apparatus server 84 may be configured to access another server or database to retrieve a list of personnel who should respond to the different types of events.

The list of personnel who should respond to a particular event may include a list of healthcare workers that are identified according to their worker classes, as discussed above. That is, the list of responding workers may indicate that, for example, a cardiac arrest of a patient requires the attendance of at least one emergency doctor or cardiac doctor, two nurses, and a nurse's assistant. This is, of course, merely one example of the many types of personnel who may be required by a particular healthcare facility to respond to a cardiac arrest. The list of responding workers may also, of course, vary for the different types of events.

Patient support apparatus server 84 (FIGS. 4 & 8), in some embodiments, is configured to consult the list of required personnel for an event and to check to see that the required personnel actually do attend to the event. In addition, in some embodiments, to the extent one or more required personnel do not attend the event, patient support apparatus server 84 is configured to send messages to the badges 142 of the missing personnel, wherein the messages summon the missing personnel to the event.

Patient support apparatus server 84 is configured to determine if any required personnel are missing based on the badges 142 of the responding personnel, as detected by the UWB transceivers 132 onboard the patient support apparatus 20 associated with the event. In other words, controller 140 detects which badges 142 are positioned within the vicinity of patient support apparatus 20 (e.g. one of space volumes 152b, c, and/or another space volume) during the event and determines what class of workers those badges 142 belong to (or sends the badge IDs 166 to patient support apparatus server 84 for it to determine the worker classes). Once the classes of the badges 142 positioned within the vicinity of patient support apparatus 20 are determined (and thus the classes of the responding personnel are known), either controller 140 or patient support apparatus server 84 consults the list of required personnel and checks to see if the healthcare workers who have actually responded meet the list of workers who are required to respond. If there are required workers who have not responded to the event, controller 140 and/or patient support apparatus server 84 may take one or more actions.

One action that controller 140 and/or patient support apparatus server 84 may take is to automatically send one or more messages to the badges 142 of workers who are of the same class as the missing personnel. For example, if only a single nurse responds to a cardiac arrest, and the healthcare facility protocol requires two nurses to respond, controller 140 and/or patient support apparatus server 84 may send a message to the badge 142 of another nurse, or multiple nurses badges 142, summoning them to the cardiac arrest. When choosing which badges 142 to send the summoning message to, patient support apparatus server 84 and/or controller 140 may be configured to automatically select the badge(s) 142 of the required type of healthcare worker(s) who is/are closest to the patient support apparatus 20 at which the event is occurring. The manner in which patient support apparatus server 84 and/or controller 140 determine which badge 142 is closest to a particular patient support apparatus 20 is discussed in greater detail below.

In some embodiments, in addition to, or in lieu of, summoning missing personnel to an event, controller 140 and/or patient support apparatus server 84 are configured to record the absence of those missing personnel in a database. The database can then be subsequently accessed by authorized individuals in order to study and/or monitor how effective the healthcare facility is at meeting its own internal guidelines regarding the handling of specified events.

In some embodiments, when patient support apparatus server 84 and/or controller 140 send messages to the badges 142 of healthcare workers-either in response to an undesired state of patient support apparatus 20 and/or in response to one or more required personnel being missing from a specified event-server 84 and/or controller 140 may be configured to choose which badges 142 to send these messages to based on several factors. One factor, as noted above, is proximity to the patient support apparatus 20. That is, in some situations, server 84 and/or controller 140 may be configured to automatically send a message to the badge 142 closest to the patient support apparatus 20 that is in the undesired state, or the patient support apparatus 20 associated with the specified event.

Another factor that server 84 and/or controller 140 may consider when deciding which badges 142 to send messages to is the patient assignments of the caregivers. That is, each caregiver is typically assigned to care for a specific subset of patients within the healthcare facility. Controller 140 and/or server 84 may therefore choose, in some situations, to send messages to only badges 142 that are associated with caregivers who are assigned to care for the patient in the particular patient support apparatus 20 that has the undesired state, or that is associated with the event. In other situations, patient support apparatus server 84 and/or controller 140 may be configured to override, or ignore, the specific patient assignments of healthcare workers, and to send messages to badges 142 of healthcare workers who may, or may not, be assigned to the patient associated with the patient support apparatus 20 that has an undesired state or is associated with the event.

For example, in some situations, patient support apparatus server 84 and/or controller 140 may send a message to a badge 142 of caregiver X because caregiver X is assigned to care for the patient assigned to the particular patient support apparatus 20 that has an undesired state or is associated with an event. In other situations, patient support apparatus server 84 and/or controller may send a message to a badge 142 of caregiver Y because caregiver Y is positioned closer to the particular patient support apparatus 20 than caregiver X, despite the fact that caregiver Y is not assigned to care for that particular patient.

Patient support apparatus server 84 and/or controller 140 may be configured to factor in the patient assignments of caregivers in different manners, depending upon priority level of an event or undesired condition. In some embodiments, server 84 and/or controller 140 are configured to send messages to the nearest badge 142 (of the appropriate class of healthcare worker), regardless of patient assignments, when there is an undesired state of patient support apparatus 20 or an associated event that is considered a high priority. In some embodiments, if the undesired state or event is an event of medium priority, controller 140 and/or patient support apparatus server 84 may be configured to send messages to badges 142 of only those caregivers who are assigned to care for the patient in that particular patient support apparatus 20. In some embodiments, if the undesired state or event is of low priority, controller 140 and/or patient support apparatus server 84 may be configured to send messages to any badges 142 (of an appropriate worker class), regardless of their patient assignments. Thus, controller 140 and/or patient support apparatus server 84 may be configured to utilize different thresholds that define different priority levels when choosing which subset of healthcare workers to send a message to.

In some embodiments, if the undesired state or event is of an even lower priority, patient support apparatus server 84 and/or controller 140 may be configured to not send any message to any badges 142 at all unless the badge 142 is positioned within a set threshold of the patient support apparatus 20. In other words, in some embodiments, patient support apparatus server 84 is configured to only notify healthcare workers who happen to walk by, or near, the patient support apparatus 20 that has the low priority state (or that is associated with a low priority event). Thus, patient support apparatus server 84 may be configured to continuously monitor the location of badges 142 and, if none are currently positioned with the threshold distance, wait until one such badge 142 is positioned therein, and then send a message to the badge 142 informing the associated healthcare worker of the undesired state of the patient support apparatus 20.

The messages sent to badges 142 by controller 140 and/or patient support apparatus server 84 may be sent in different manners. Controller 140 may send messages to badge 142 directly using one or more of the UWB transceivers 132; it may send messages directly to badge 142 using a Bluetooth transceiver 128; and/or it may send messages indirectly by first sending a message to patient support apparatus server 84 or badge server 94 (using network transceiver 96), and then having that server forward the message to the badge via the badge's network transceiver 164

It will be appreciated that, although the foregoing discussion of sending messages to healthcare workers regarding the undesired states of patient support apparatus 20 and/or regarding events has focused on sending messages to badges 142, patient support apparatus server 84 and/or controller 140 may be configured to alternatively, or additionally, send messages to an electronic device 98 of a caregiver. In other words, instead of notifying a caregiver of an undesired state of, or event regarding, patient support apparatus 20 via badge 142, patient support apparatus 20 and server 84 may notify the caregiver via his or her electronic device 98. In order to send a message to the caregiver's electronic device 98, server 84 consults another server on network 80, or a database accessible thereto, that associates healthcare workers with specific electronic devices 98. Server 84 uses that information to determine which specific electronic device 98 the messages should be sent to, and then forwards the message using access points 82 (or, instructs patient support apparatus 20 which specific electronic device 98 to forward the message to, and controller 140 then sends the message to that specific electronic device 98 using one of its onboard transceivers).

Controller 140 of patient support apparatus 20 may also be configured to automatically associate and disassociate patient support apparatus 20 from other devices 100 besides badges 142. Once such device 100 is depicted generically in FIGS. 4 and 5. Device 100 includes, at a minimum, a UWB transceiver 178 and a unique ID 180 (FIG. 5). Device 100 may also, depending upon the particular device 100, include a device controller 182 and/or one or more sensors 184. Device 100 may also include additional structures beyond those shown in FIG. 5. In some embodiments, UWB transceiver 178 and ID 180 are incorporated into a UWB tag that is separable from, but attachable to, the device 100. The tags may be of the same type discussed above that are worn by a patient, and such tags may take on any of the forms of the tags described in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which is incorporated herein by reference. In other embodiments, UWB transceiver 178 and unique ID 180 may be built-into the device 100 and not incorporated into a separable tag.

Controller 140 of patient support apparatus 20 automatically associates patient support apparatus 20 with, and disassociates patient support apparatus 20 from, one or more devices 98 or 100 by repetitively determining the relative location of UWB transceiver 178 of each of the various devices 98, 100 with respect to patient support apparatus 20. As will be discussed in greater detail below, controller 140 may associate the device 98, 100 to patient support apparatus 20 if it moves inside of one of the space volumes 152 (also discussed in more detail below), and disassociate the device 98, 100 if it moves outside of one of these space volumes 152. The types of devices 98, 100 that may be incorporated into the communication system described herein include, but are not limited to, any one or more of the following: another patient support apparatus 20, a UWB-equipped smart phone, a UWB-equipped mobile computer, an infusion pump, a vital sign sensor, an exercise device, a heel care boot, an IV stand and/or pole, a ventilator, a DVT pumps, a patient monitor (e.g. a saturated oxygen (SpO₂) monitor, an EKG monitor, a vital sign monitor, etc.), a patient positioning devices (e.g. a wedge, turning device, pump), an ambient sensor (e.g. air temperature, air flow, light, humidity, pressure, altitude, sound/noise), a mattress 42, a portable exit detection sensor, an attachable nurse call device, an incontinence pad or one or more sensors adapted to detect patient incontinence, a Holter device adapted to monitor and record a patient's heart signals, a patient ID tag or bracelet worn by the patient that identifies the patient, a caregiver tag or ID bracelet worn by a caregiver that identifies the caregiver, a patient temperature management device (or associated device, such as a one or more hoses, thermal wraps, etc.), one or more mobility assistance devices that a patient may be expected to use, and/or still other types of devices 100.

In those embodiments where devices 100 include an infusion pump, patient support apparatus 20 and patient support apparatus server 84 may be configured to carry out any of the functions associated with the infusion pump that are described in commonly assigned U.S. patent application Ser. No. 63/349,369 filed Jun. 6, 2022, by inventors Krishna Bhimavarapu et al. and entitled COMMUNICATION SYSTEM FOR PATIENT SUPPORT APPARATUSES, the complete disclosure of which is incorporated herein by reference. In those embodiments where devices 100 include a portable exit detection sensor, another patient support apparatus, and/or an attachable nurse call device, patient support apparatus 20 and patient support apparatus server 84 may be configured to carry out any of the functions associated with the portable exit detection sensors, nurse call devices, and secondary patient support apparatuses disclosed in commonly assigned U.S. patent application Ser. No. 63/352,061 filed Jun. 14, 2022, by inventors Jerald Trepanier et al. and entitled COMMUNICATION SYSTEM FOR PATIENT SUPPORT APPARATUSES, the complete disclosure of which is incorporated herein by reference.

Either in addition to, or in lieu of, of the automatic association of some devices 98 and/or 100 with patient support apparatus 20, controller 140 may be configured to allow one or more specific devices 98, 100 to be associated with patient support apparatus 20 through a manual process. That is, some devices 98, 100 may be associated with patient support apparatus 20 in a manual process, some devices may be associated in an automatic process, and some devices 98, 100 may be associated in both a manual and an automatic process. For the manual process, the caregiver manually informs patient support apparatus 20 that a particular device 98, 100 should now be associated with that patient support apparatus 20. This manual process may be accomplished in different manners. For example, in some embodiments, the device 98 and/or 100 and patient support apparatus 20 may include near field transceivers that, when positioned within close proximity (e.g. several inches) of each other, exchange information that establishes that that particular device 98, 100 should be associated with that particular patient support apparatus. Further details regarding the use of near field transceivers for associating devices 100 to a patient support apparatus 20 are disclosed in commonly assigned U.S. patent application Ser. No. 63/352,061 filed Jun. 14, 2022, by inventors Jerald Trepanier et al. and entitled COMMUNICATION SYSTEM FOR PATIENT SUPPORT APPARATUSES, the complete disclosure of which has already been incorporated herein by reference. Other types of manual associations processes may also, or alternatively be used, some of which are also disclosed in the aforementioned '061 application.

For the automatic association process, as noted previously, controller 140 is configured to automatically associate at least some devices 98 and/or 100 with patient support apparatus 20 based upon the proximity of those devices 100 to patient support apparatus 20. This automatic association may be accomplished by the device 98, 100 moving inside of a predetermined volume of space 152 defined with respect to patient support apparatus 20 (e.g. one of space volumes 152a-c, or another space volume; FIG. 6). Once inside the space, controller 140 automatically associates the device 98 and/or 100 with patient support apparatus 20.

Controller 140 is also configured to automatically disassociate one or more devices 98 and/or 100 from patient support apparatus 20. The automatic disassociation may be accomplished by the device 98, 100 moving outside of the same predetermined volume of space 152. Alternatively, controller 140 may use modified volumes of space-such as, but not limited to, larger space volumes-when automatically determining whether to disassociate one of these devices 98, 100 from patient support apparatus 20. In other words, once a device 98, 100 has been determined to be positioned inside of a particular volume of space, such as space volumes 152a-c (and any additional association conditions are met, if there are any), and controller 140 has associated the device 98, 100 with patient support apparatus 20, controller 140 may thereafter increase the size of—and/or otherwise change one or more dimensions of—the volume of space when determining whether to disassociate the device 98, 100. In this manner, the volumes of space 152 may have a sort of hysteresis aspect wherein a device 98, 100 has to be positioned inside of a smaller space volume in order to be associated with patient support apparatus 20, but thereafter can only be disassociated if it moves outside of a larger sized volume of space. In still other embodiments, the dimensions of one or more of the volumes of space are the same for both association and disassociation purposes.

Once a device 98, 100 is associated with patient support apparatus 20, it thereafter remains associated with patient support apparatus 20 until it moves outside of a volume of space, such as space 152b of FIGS. 4 & 6, at which point controller 140 disassociates the device 98, 100 from patient support apparatus 20. Controller 140 repetitively determines and monitors the position of device 98, 100 while it is within communication range of controller 140. Specifically, controller 140 uses UWB transceivers 132, which communicate with UWB transceiver 178 onboard device 100 (or UWB transceiver 218 onboard an electronic device 98), to repetitively determine the relative position of device 100 (or 98) with respect to patient support apparatus 20. Controller 140 compares this relative position to a particular space volume 152 and, if it is outside of the space volume 152, it automatically disassociates device 100 (or 98) from patient support apparatus 20. On the other hand, as long as the position of device 100 (or 98) remains inside of the space volume 152, controller 140 maintains the association of device 100 (or 98) with patient support apparatus 20. As noted previously, the dimensions of the space volume 152 that are used for the association/dissociation process of a device 98 or 100 may be the same as, or different from, the dimensions of space volume 152 that are used for the association/dissociation process of locator units 60 and/or badges 142. The dimensions of space volume 152 that are used for the association/disassociation process of a particular device 98 and/or 100 may also be the same as, or different from, the dimensions of space volume 152 that are used for the association/disassociation process of other particular devices 98 and/or 100.

Electronic device 98 (FIG. 5) also includes a controller 220 that oversees the operation of the electronic device 98. In some embodiments of patient support apparatus 20, it is not necessary for electronic device 98 to execute any specialized software application for it to be able to use UWB transceiver 218 to communicate with UWB transceivers 132 onboard patient support apparatus 20. Indeed, is it not necessary in some embodiments for the electronic device 98 to even pair with patient support apparatus 20. Thus, in some embodiments, patient support apparatus 20 is able to detect the location of a UWB-equipped smart phone 98, UWB-equipped tablet computer 98, or other UWB-equipped electronic device 98 without the device having to execute any software application that is tailored for communication with patient support apparatus 20, or that requires pairing with patient support apparatus 20. Instead, the smart phone, table, or other electronic device 98 merely has to have their UWB transceiver 218 turned on such that it periodically advertises its presence to any UWB receivers (e.g. UWB transceivers 132 of patient support apparatus 20) that are within communication range. From this information, controller 140 is able to receive a unique ID 222 from the device 98 and to determine its location relative to patient support apparatus 20, and this can be done without requiring the user of the device 98 to take any pairing action, or other action specific to patient support apparatus 20.

In some embodiments, controller 140 forwards the ID 222 from a detected electronic device 98 to server 84. Server 84 includes, or has access to, a database that correlates the IDs 222 from to users or owners of the corresponding electronic devices 98. From this database, server 84 is able to determine whose smart phone 98, tablet computer 98, or other UWB-equipped computer 98 it has detected. This information may then be shared with patient support apparatus 20 and/or with one or more other devices (e.g. computer 168, display device 98, etc.).

Display device 56 (FIG. 5) includes a display controller 192 and a display 194. Display device 56 differs from the other devices 100 and/or badges 142 discussed herein in that it does not need to include a UWB transceiver, such as is found in these other devices. This is because patient support apparatus 20 is not configured to determine the location of display device 56. Instead, controller 140 of patient support apparatus 20 is configured to use UWB transceivers 132 to determine the location of the locator unit 60 to which the display device 56 is coupled. If the patient support apparatus 20 (or a reference point on it) and locator unit 60 are not both positioned within the space volume 152a, the controller 140 will not send data to be displayed on display device 56 to the locator unit 60. If the locator unit 60 and patient support apparatus 20 (or a reference point on it) are both positioned within the space volume 152a, controller 140 is configured to be able to send data to be displayed on the coupled display device 56 by forwarding the data to be displayed to the associated locator unit 60, which then forwards it to display device 56 via video port 120 and video cable 110. In some embodiments, controller 140 forwards the data to be displayed on display device 56 to the associated locator unit 60 by using Bluetooth transceiver 128, while in other embodiments it may use a UWB transceiver 132, or another type of transceiver. The forwarding of data to be displayed on display device 56 is a direct forwarding of data from patient support apparatus 20 to locator unit 60, and from there to display device 56, with no other intermediaries, in at least some embodiments.

Because display device 56 need not include a UWB transceiver, display device 56 may be a conventional television, computer monitor, or other conventional device, that is capable of displaying the video signals that are transmitted over video cable 110. In addition, it is not necessary for display device 56 to execute any specialized software app that authenticates communication with the coupled locator unit 60 and/or with patient support apparatus 20. Instead, once controller 140 determines that locator unit 60 is within space volume 152a (or vice versa), controller 140 need not perform any additional security protocols regarding display device 56. Indeed, controller 140 does not need to be able to perform any communications with display device 56 other that forwarding data to be displayed to the associated locator unit 60. In this manner, display device 56 need not know anything about locator unit 60 and/or patient support apparatus 20. It merely displays the video signal coming from video cable 110, and therefore does not need a software app that is specialized to patient support apparatus 20 and/or to locator unit 60 and/or to UWB communications.

It should also be noted that the display of data on display 52 and/or display device 56 from an associated device 100 is carried out by patient support apparatus 20 automatically. That is, for example, when a user has configured patient support apparatus 20 to display data from a device 100 on a display device 56, controller 140 automatically forwards this data to the associated display device 56. For example, if device 100 is a UWB-tagged heart rate monitor and a patient coupled to device 100 is wheeled into a bay area of a patient room that includes a display device 56 coupled to a locator unit 60, controller 140 is configured to automatically start displaying the patient's heart rate information on that display device 56 as soon as patient support apparatus 20 completes the following two association processes: (1) the association between patient support apparatus 20 and the locator unit 60 to which display device 56 is coupled, and (2) the association between patient support apparatus 20 and the heart rate monitor. In this manner, the caregiver sees the patient's heart rate data displayed on the display device 56 within seconds after moving the patient support apparatus 20 into the bay area, and the caregiver doesn't need to connect any cables, press any buttons, or take any other actions, in order for the heart rate data to be displayed on display device 56. Similarly, when the patient support apparatus 20 moves out of the bay area, the display of the patient's data on the display device 56 automatically terminates. This automatic termination is carried out after patient support apparatus 20 moves outside of the space volume 152a associated with the locator unit 60 to which the display device 56 is connected. Once outside of this space volume 152a, controller 140 disassociates patient support apparatus 20 from that locator unit 60 and its associated display device 56, and once this disassociation occurs, it stops sending data to be displayed on display device 56.

As another example of the automatic display of device data on a display, if a caregiver approaches a patient support apparatus 20 while carrying a portable electronic device 98—such as a smart phone or tablet computer that includes a display, a UWB transceiver, and the appropriate software app—controller 140 is adapted to automatically start forwarding the data to the electronic device 98 for display on its screen. In this manner, a caregiver that brings his or her portable electronic device 98 within space volume 152b (or another space volume defined for such devices 98) can have that patient's heart rate data (and/or other data, such as from another device 100) automatically displayed on his/her device 98. When the caregiver moves his or her portable electronic device 98 outside of the space volume 152b, the display of that patient's data automatically ceases because controller 140 automatically disassociates patient support apparatus 20 from the portable electronic device 98 and stops sending it data to be displayed. If the caregiver then moves to another patient's patient support apparatus 20 and steps inside the space volume 152b of that patient support apparatus, he or she will automatically see the data from the devices 100 associated with that patient displayed on his or her portable electronic device 98.

In some embodiments, controller 140 may be configured to display data from a device associated with patient support apparatus 20, and/or process the data from the associated device, in any of the manners disclosed in commonly assigned U.S. patent application Ser. No. 63/306,279 filed Feb. 3, 2022, by inventors Madhu Sandeep Thota et al. and entitled COMMUNICATION SYSTEM FOR PATIENT SUPPORT APPARATUSES, the complete disclosure of which is incorporated herein by reference.

In some embodiments, patient support apparatus 20 is configurable by a user (e.g. via control panel 54a) to control what information, if any, patient support apparatus 20 will send to an associated display device 56 (FIG. 5), to patient support apparatus server 84, and/or to EMR server 92. Thus, a user can instruct patient support apparatus 20, for example, to send blood pressure readings, breathing rate readings, and pulse rate readings from devices 100 that are vital sign sensors (and that are associated with patient support apparatus 20) to EMR server 92, but not status data from a device 100 that doesn't generate data for recordation in EMR server 92, such as a badge 142, a recliner, a portable nurse call device, or still other devices. Patient support apparatus 20 may also be configured by the user to route data for displays 52 and/or 56 and/or one or more servers on network 80 in still other manners.

Locator units 60 are configured to send a message to patient support apparatuses 20 indicating whether or not a display device 56 (FIGS. 4-5) is coupled thereto via video port 120. In such embodiments, when controller 140 of patient support apparatus 20 receives the signal indicating that a display device 56 is coupled to an associated locator unit 60, it is configured to consult the customized user settings to determine what data, if any, to send to that locator unit 60 for forwarding to the display device 56 coupled to video port 120. It bears noting that, when patient support apparatus 20 associates itself with a particular locator unit 60 that has a display device 56 coupled to its video port 120, it is not necessary for the display device 56 to be positioned within space volume 152a in order for controller 140 to send data to be displayed on the display device 56. Instead, controller 140 is configured to only check whether the locator unit 60 is associated with patient support apparatus 20 or not. If it is, controller 140 may send (depending upon the user settings) data to the locator unit 60 for forwarding to the display device 56. If locator unit 60 is not associated with patient support apparatus 20, then controller 140 does not send any data to the locator unit 60 for displaying on display device 56.

In addition to allowing a user to customize what data, if any, that is sent to a display device 56, controller 140 is further configured to allow a user to customize what data is sent to different types of display devices 56. Thus, controller 140 may be programmed to allow a user to send a first set of data to a display device 56 that is coupled to an associated locator unit 60 and a second set of data, different from the first set of data, to a different display, such as, for example, display 52 of patient support apparatus 20 and/or another display, such as, but not limited to, ones that are incorporated into electronic device 98 (e.g. conventional smart phones, laptop computers, tablet computers, smart TVs and/or smart monitors) that are either in communication with patient support apparatus server 84 and/or that are positioned within space volume 152, as determined by one or more UWB transceivers coupled to them that are adapted to communicate with UWB transceiver 132 of patient support apparatus 20. It will be appreciated, that in such cases, the conventional smart phone, laptop computer, tablet computer, or other type of display device will include a software app that oversees the UWB communications with patient support apparatus 20 and that controls the display of the data on the display (i.e. screen) of the display device. The software app includes one or more security features built into it that only allow authorized users to have this data displayed on the display device, thereby preventing unauthorized users from viewing this data on their own smart phone, laptop computer, tablet computer, etc. In some embodiments, controller 140 is configured to execute a security check protocol with the display device to authenticate the display device prior to sending it any data for displaying thereon.

Patient support apparatus 20 may include one or more screens that are displayable on display 52 that allow a user to customize the data from associated devices 100 that is shown on display 52 and/or display devices 56. In addition, patient support apparatus 20 is configured to allow the user to change the format and/or layout in which the selected data is to be displayed.

FIGS. 4 and 6 illustrate several of the different volumes of space 152 that controller 140 may use when carrying out the functions described herein. As was noted, in some embodiments, space volumes 152a-c are defined with respect to patient support apparatus 20 and therefore move as patient support apparatus 20 moves. Space volume 152a, which may be the smallest of the space volumes 152, is generally used for the automatic association and disassociation between patient support apparatus 20 and a locator unit 60. Space volume 152b is generally used for the automatic association and disassociation between patient support apparatus 20 and a variety of different devices 100 that are typically used in close proximity to the patient. Space volume 152c is generally used by controller 140 for the automatic association and disassociation between patient support apparatus 20 and badges 142. It will be understood that controller 140 may utilize other space volumes 152 than the three shown in FIGS. 4 and 6.

Space volume 152c is generally sized such that it encompasses substantially all of the room 58 in which patient support apparatus 20 is positioned, or at least that portion of the room in which a healthcare worker with a badge 142 is expected to be present, particularly while the worker attends to the patient assigned to patient support apparatus 20 and/or to patient support apparatus 20 itself. In some situations, space volume 152c may be large enough to extend into adjacent rooms 58, but this will not affect the proper association of a badge 142 with patient support apparatus 20 so long as access to those adjacent rooms cannot be obtained without the healthcare worker first exiting from space volume 152b before traveling to those adjacent rooms. In other words, space volume 152c may be advantageously defined such that, when the person's badge 142 is moved out of room 58 through a doorway, the badge 142 will necessarily move outside of space volume 152c, thereby causing it to become disassociated from the patient support apparatus 20 before the badge 142 enters the space volume 152c associated with another patient support apparatus 20.

In some embodiments, any of space volumes 152a-c may be defined with one or more static dimensions. In other embodiments, patient support apparatus 20 and/or patient support apparatus server 84 may be configured to allow authorized individuals to change one or more dimensions of space volumes 152a-c. Still further, in some embodiments, space volumes 152a-c may have variable dimensions based upon the specific room, bay, or other location, in which patient support apparatus is currently positioned. In these embodiments, controller 140 may utilize a table stored in memory 134 that defines the dimensions of any of space volumes 152a-c based on the current location of patient support apparatus 20. The location of patient support apparatus 20 may be determined by controller 140 from the locator unit ID 122 that it receives from an associated locator unit 60, and/or it may be derived from information received from patient support apparatus server 84.

FIG. 6 illustrates an example of an arbitrary healthcare facility room 58 in which is positioned a patient support apparatus 20, a device 100, and a badge 142. FIG. 6 also illustrates first volume of space 152a, second volume of space 152b, and third volume of space 152c. First volume of space 152a, as has been discussed, is used to determine whether patient support apparatus 20 should associate or disassociate itself from the locator unit 60 that is positioned adjacent to, or inside of, first volume of space 152a. Second volume of space 152b is used by controller 140, as has been noted, to determine whether to associate or disassociate a device 100 with patient support apparatus 20. Third volume of space 152c may be used by controller 140 to determine whether to associate or disassociate one or more badges 142 with patient support apparatus 20. Second volume of space 152b is generally smaller than third volume of space 152c because it is used by controller 140 to associate devices 100 that are typically placed in close proximity to patient support apparatus 20 when used in the care of the patient (as opposed to a badge 142, which may be positioned further away from patient support apparatus 20, such as when a healthcare walks about the interior or a room). It can therefore be seen that controller 140 of patient support apparatus 20 may be configured to use multiple different volumes of space 152a-c for determining whether to associate a device 100 with patient support apparatus 20—one for locator units 60, one for some devices 100, one for badges 142, and, in some embodiments, still another one (or ones) for other types of devices 100 and/or 98.

As shown in FIG. 6, first volume of space 152a is dimensioned so that it will encompass fixed locator unit 60 when patient support apparatus 20 is positioned within about 5 to 10 feet of a locator unit 60. Second volume of space 152b encompasses patient support apparatus 20 and a relatively small amount of space surrounding patient support apparatus 20. This small amount of space around the perimeter of patient support apparatus 20 may be defined with the goal of encompassing IV poles, infusion pumps, or other devices that may be used in the care of the patient, but that may not necessarily be directly supported on patient support apparatus 20. Second volume of space 152b may also be defined such that it is unlikely to encompass devices 100 that are being used on a patient assigned to another patient support apparatus 20 that may be positioned nearby. Third volume of space 152c encompasses a substantial portion of room 58. It will, of course, be understood that the sizes and dimensions of volumes of space 152a-c may vary from what is shown in the attached drawings.

As shown in FIG. 6, a fourth volume of space 152d may also be defined for locator units, such as unlinked locator units 60a, that are positioned in hallways, and/or in other areas outside of patient rooms 58. Space volumes 152d may be sized to provide more generalized locations of patient support apparatuses 20 (and the devices associated therewith), such as being located in a particular hallway, or portion of a hallway, a particular storage area, etc. In some embodiments, space volume 152d is used by controller 140 when determining whether to associated with an unlinked locator unit 60a. In some embodiments, the dimensions of space volumes 152d for each unlinked locator unit 60a are stored within a memory of each unlinked locator unit 60a and transmitted to patient support apparatus 20 when patient support apparatus 20 is within communication range. Alternatively, the dimensions of space volume 152d may be stored onboard patient support apparatus 20 and consulted by controller 140 based upon the ID 122 received from an unlinked locator unit 60a.

It will be understood that the dimensions of space volume 152d, like those of space volumes 152a-c, may be variable, customizable, location-dependent, device-dependent, and/or different from what is shown in FIG. 6. It will also be understood that, although the accompanying drawings all depict all of the space volumes 152a-d as having rectangular shapes, these shapes may be varied, including shapes that are all curved and/or shapes that have a combination of curved and straight boundaries. It will also be understood that the volumes of space 152a-c may be alternatively referred to as threshold distances from patient support apparatus 20. If the threshold distance has a constant value in all directions from a measurement point of the patient support apparatus 20, the volume of space 152 will define a sphere. If the threshold distance has a non-constant value in one or more different directions from the measurement point of the patient support apparatus 20, the volume of space 152 will define a non-spherical shape.

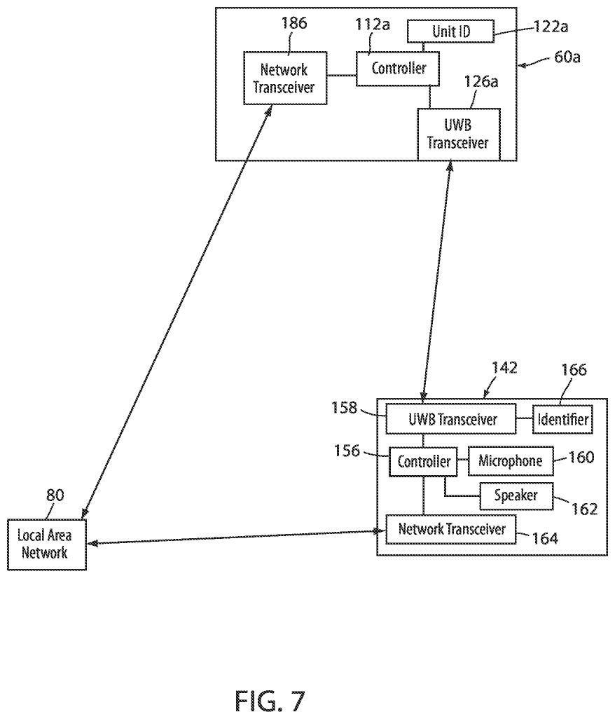

FIG. 7 illustrates an example of the internal components of an unlinked locator unit 60a (as well as a badge 142 and local area network 80). Unlinked locator unit 60a includes a number of components that are the same as those found in linked locator units 60. Those common components are labeled with the same reference number followed by the letter “a.” Components of unlinked locator units 60a that are not found in linked locator units 60 are provided with a new reference number.

Unlinked locator unit 60a includes a controller 112a, a UWB transceiver 126a, a unit ID 122a, and, in some embodiments, a network transceiver 186. Network transceiver 186 is, in at least one embodiment, a WiFi transceiver adapted to be able to communicate with network 80 via wireless access points 82. Controller 112a is adapted to determine the location of badges 142 that are positioned within range of UWB transceiver 126a. In some embodiments, unlinked locator unit 60a includes multiple UWB transceivers 126a, while in other embodiments, unlinked locator unit 60a may include only a single UWB transceiver 126a. Regardless of how many UWB transceivers 126a unlinked locator unit 60a includes, controller 112a is configured to use UWB transceiver(s) 126a to measure the distance between itself and any badges 142 that are positioned within range of unlinked locator unit 60a. In some situations, such as where unlinked locator unit 60a includes only a single UWB transceiver 126a, controller 112a may only be able to determine a distance between itself and the badge 142, rather than a three-dimensional position. In other situations, such as where unlinked locator unit 60a includes multiple UWB transceivers 126a, controller 112a may be able to determine a three-dimensional position of a badge 142 relative to unlinked locator unit 60a.

Regardless of the 1-dimensional, 2-dimensional, or 3-dimensional position determination of a badge 142, controller 112a is configured to send a message to patient support apparatus server 84 via network transceiver 186 that identifies the detected location of any badge 142 with respect to unlinked locator unit 60a. This message also identifies the unique identifier 122a of the unlinked locator unit 60a. Patient support apparatus server 84 is therefore able to determine the general location of the badge 142 within the healthcare facility using the stored positions of each locator unit 60 within the healthcare facility.

In addition to tracking the position of badges 142 as they move through the healthcare facility, unlinked locator units 60a may also be provided within a healthcare facility in order to provide location information to patient support apparatuses 20 as they are moved outside of the rooms within the healthcare facility. Patient support apparatus 20, in some embodiments, is adapted to communicate with UWB transceiver 126a of unlinked locator units 60a in the same manner that it communicates with UWB transceiver 126 of linked locator units 60. This enables patient support apparatus 20 to determine its relative position with respect to the unlinked locator unit 60a (which, in some embodiments, is a three-dimensional position). Controller 140 can therefore determine its position within the healthcare facility, and/or forward the locator ID 122a from the unlinked locator unit 60a to patient support apparatus server 84, and patient support apparatus server 84 can utilize this information to determine the current location of patient support apparatus 20 within the healthcare facility.

In some embodiments, controller 140 of patient support apparatus 20 is configured to communicate with unlinked locator units 60a in a manner different from the manner that it communicates with linked locator units 60. In some such embodiments, controller 140 may determine its direction of movement relative to an unlinked locator unit 60a as the patient support apparatus 20 moves by the unlinked locator unit 60a, while controller 140 may only determine its position relative to a linked locator unit 60. In other words, in some embodiments, controller 140 determines the direction of movement of patient support apparatus 20 relative to an unlinked locator unit 60a, but does not determine the direction of movement of patient support apparatus 20 relative to a linked locator unit 60.

Patient support apparatus server 84 repetitively receives position information about all of the badges 142 that are currently positioned in communication range of the UWB transceivers 132 onboard the multiple patient support apparatuses 20 within the healthcare facility. Patient support apparatus server 84 may also receive position information from unlinked locator units 60a whose UWB transceivers 126a (FIG. 7) are positioned within communication range of one or more badges 142. Patient support apparatus server 84 is therefore repetitively provided with updates about the current location of all badges 142 within the healthcare facility that are within UWB communication range of a patient support apparatus 20 and/or an unlinked locator unit 60a. From this information, patient support apparatus server 84 is able to determine which badge(s) 142 are positioned closest to any patient support apparatus 20 at any given time.

It will also be understood that patient support apparatus server 84, in some embodiments, may also receive an utilize additional position information about the current locations of badges 142. That is, in addition to the position information of badges 142 as determined by their UWB communications with locator units 60 and/or with patient support apparatuses 20, patient support apparatus server 84 may also receive and utilize position information of badges 142 that is determined by badge server 94 using conventional WiFi location methods (e.g. triangulation, signal strength measurements, etc.). In such embodiments, patient support apparatus server 84 is configured to determine the location of badges 142 using two independent location-determination systems: a UWB-based location system and a WiFi-based location determination system. The locations determined from both of these systems may be combined by patient support apparatus server 84 to determine the location of badges 142 with better accuracy and/or with a broader range than when using only a single one of these location systems.

FIG. 8 illustrates an overview of the communication system of the present disclosure, including various types of data that may be stored within different servers on a healthcare facility's computer network 80. As was noted previously, EMR server 92 stores the electronic medical records of individual patients, and this data may include individual care plans for each patient, such as shown in table 322. ADT server 102 stores data indicating the current location within the healthcare facility for each patient, such as is indicated in table 320. Badge server 94 stores badge IDs (e.g. identifiers 166), the healthcare workers associated with those badge IDs, and whether or not the healthcare worker is currently logged into the system (e.g. whether the worker is currently using the system or not), as indicated by table 326. Caregiver assignment server 104 stores the rooms—and/or bays within rooms—of the patients that each caregiver is assigned to care for, as shown in table 324. Patient support apparatus server 84 stores a variety of data, some of which is illustrated in patient support table 330 of FIG. 8.

Patient support table 330 includes a column of locator IDs 122, a column of rooms, a column of bed status information, a column indicating the presence or absence of caregivers, a column indicating what devices 100 are associated with each patient support apparatus 20, a column of patient support apparatus IDs 130, and a column of function identifiers that indicate what functions have been performed using each patient support apparatus 20. The locator IDs 122 are forwarded by each patient support apparatus 20 to patient support apparatus server 84 after patient support apparatus 20 associates itself with a particular locator unit 60. The rooms corresponding to each locator ID are determined automatically by patient support apparatus server 84 using its onboard memory, which includes the surveying information from the installation of the locator units 60 (which, in turn, identifies the rooms and/or bays of each locator unit 60).

Patient support apparatus server 84 determines whether a caregiver is currently positioned nearby to each patient support apparatus 20 based upon whether the caregivers' badge 142 is currently associated with that patient support apparatus 20 or not. In other words, when controller 140 associates patient support apparatus 20 with a badge 142, it forwards this association determination (and the badge ID 166) to patient support apparatus server 84, which uses the ID 166 to determine the caregiver's identity. As long as the badge 142 remains associated with patient support apparatus 20, patient support apparatus server 84 considers the caregiver to be present within the vicinity of the patient support apparatus 20. Once controller 140 disassociates a badge 142 from patient support apparatus 20, it also sends a message to patient support apparatus server 84 and patient support apparatus server 84 then concludes that the caregiver is no longer present within the vicinity of the patient support apparatus 20.

As shown in table 330 of FIG. 8, patient support apparatus server 84 also receives data from the device(s) 100 that are currently associated with each patient support apparatus 20, as shown in the column labeled “Assoc. Device Status.” As has been described herein, patient support apparatus server 84 may forward some or all of this data to EMR server 92, one or more electronic devices 98, one or more other devices, and/or store this data. Table 330 also includes the patient support apparatus IDs 130 of each patient support apparatus 20. Still further, table 330 includes a column containing data identifying what functions have been performed with each patient support apparatus 20. This functional data is sent by controller 140 to patient support apparatus server 84 in response to a user carrying out one or more functions of patient support apparatus 20, as was descried previously.

It will be understood that EMR server 92, badge server 94, caregiver assignment server 104, and ADT server 102 (FIGS. 4 and 8) may all be conventional and/or commercially available servers.

In some embodiments, when patient support apparatus 20 and/or server 84 associate a device 100 and/or badge 142 with a particular patient support apparatus 20, controller 140 and/or server 84 are configured to inform medical personnel (via electronic devices 98) that the device is associated with a particular patient support apparatus 20 and/or with a particular patient assigned to that patient support apparatus 20. In this manner, data from the device can be correlated with a particular patient, room location, and/or patient support apparatus 20. Patient support apparatus 20 may therefore be configured to automatically forward data from the associated medical devices to server 84 after the devices become associated with patient support apparatus 20. Such data may, in turn, be automatically forwarded by server 84 to EMR server 92 for entry into the corresponding patient's electronic medical record. In this manner, once controller 140 associates a device with the patient support apparatus 20, data from the device can be automatically recorded in that patient's particular electronic medical record without requiring the caregiver to either manually associate the device with the patient and/or with the patient support apparatus 20 assigned to that patient. In other words, because controller 140 automatically determines that the device is positioned within a corresponding space volume 152, it is not necessary for a caregiver to take any manual steps to ensure that data from the device is forwarded to the proper corresponding patient's electronic medical record because patient support apparatus 20, along with server 84, automatically determine the correct patient associated with that medical device.

In those situations where controller 140 and/or server 84 determine the identity of a specific patient assigned to a particular patient support apparatus, controller 140 and/or server 84 communicates with ADT server 102 (FIG. 8). ADT server keeps track of which patients are assigned to which rooms and/or which bays of rooms with multiple patients. In order for controller 140 or server 84 to determine which patient is assigned to a particular patient support apparatus 20, they need only to determine which room and/or bay that particular patient support apparatus 20 is currently located in. This is accomplished through communications with an associated locator unit 60, as discussed above. In brief overview, controller 140 either sends the locator unit ID 122 of an associated locator unit 60 to patient support apparatus server 84, which then looks up the room and/or bay location of that locator unit ID 122, or controller 140 consults an onboard listing of what rooms and/or bays correspond to each particular locator unit 60. In either case, the room or bay number of a particular patient support apparatus 20 is determined. After determining which room or bay a particular patient support apparatus is located in, ADT server 102 is consulted to determine the particular patient assigned to that particular room or bay (and thus that particular patient support apparatus 20).

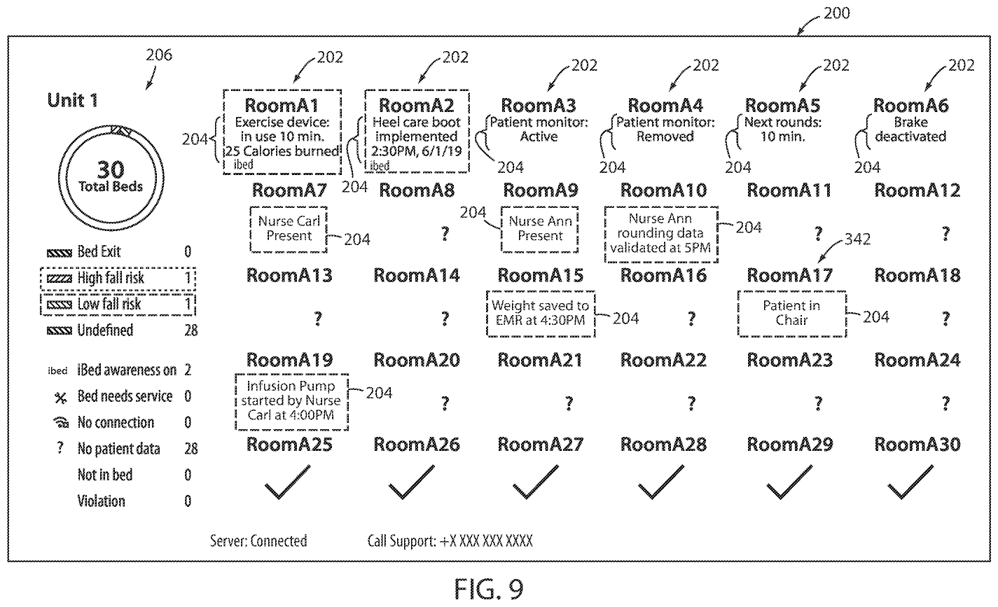

FIG. 9 illustrates one example of the type of data that patient support apparatus 20 may be configured to send to one or more electronic devices 98. More specifically, FIG. 9 illustrates one example of a dashboard screen 200 that patient support apparatus server 84 may be configured to display on one or more electronic devices 98. Dashboard screen 200 is a multi-room dashboard display that indicates the status of patient support apparatuses in a plurality of rooms of a healthcare facility, as well as information regarding the location and activities of healthcare workers with badges 142. The electronic device 98 on which dashboard screen 200 may be displayed includes a WiFi connection for communicating with an access point 82 of network 80, and thus is able to receive status data from patient support apparatus server 84.

Dashboard screen 200 includes a plurality of individual room numbers 202 and corresponding status messages 204 indicating one or more parameters about the status of the patient assigned to that room, the patient support apparatus 20 in that room, the caregiver assigned to the patient in that room, and/or any other workers with badges 142 who may be present in the room and/or who may have performed one or more functions on that patient support apparatus 20. The information that is displayable on screen 200 includes any information regarding the patient support apparatus 20 positioned in that room, as well as any information about badges 142, devices 100, and/or locator units 60 that are associated with that patient support apparatus. Thus, for example, as shown in FIG. 9, status messages 204 may indicate that an exercise device 100 has been in use for ten minutes and the patient has burned 25 calories (room A1), that a heel care boot 100 has been applied to a patient as of 2:30 PM on Jun. 1, 2019 (room A2); that a patient monitor 100 is active (room A3); that a patient monitor has been moved out of proximity of a patient support apparatus 20 (e.g. out of space volume 152b) (room A4); that a caregiver is expected to perform a rounding duty for a patient within a specific time period (room A5); that a brake on patient support apparatus 20 is deactivated (room A6); that nurse Carl is currently present in room A7; that Nurse Ann is currently present in room A9; that Nurse Ann's rounding data for room A10 was validated at 5 PM; that the patient's weight in room A15 was sent to EMR server 92 at 4:30 PM; that the patient is currently sitting in a recliner in room A17 (where the recliner includes a patient presence sensor and is a UWB-equipped device 100); and that an infusion pump was started by Nurse Carl at 4 PM in room A19. Still other types of information may be displayed on dashboard screen 200, including additional details regarding the status messages 204. Such additional details may be displayed in response to, for example, a user tapping on, or otherwise selecting a particular one of the rooms 202 on screen 200.

Screen 200 may also include a summary status area 206 that lists certain collective data statistics about patient support apparatuses 20, devices 100, badges 142, and/or patients. For example, summary area 206 may indicate how many patient support apparatuses 20 are currently issuing an exit alert (detected by exit detection system 136); how many patients are classified as high or low fall risks (or whose fall risk categorization has not been completed); how many patient support apparatuses 20 have their onboard monitoring system 138 activated; how many patient support apparatuses 20 are in need of service; how many are not connected to network 80, and/or other data. As noted, tapping on, or otherwise selecting, any individual room 202 of screen 200 may cause the electronic device 98 to display additional information about the status of the patient, patient support apparatus 20, and/or any device(s) 100 and/or badges 142 that are positioned in that room.

In some embodiments, controller 140 is configured to send messages to patient support apparatus server 84 indicating one or more functions that have been performed on patient support apparatus 20, and/or indicating that one more functions that have not been performed on patient support apparatus 20. The content of these messages may be displayed on dashboard screen 200 and/or it may be saved in a memory accessible to server 84. Alternatively, or additionally, such messages may be forwarded to one or more caregiver badges 142 and/or other electronic devices 98 informing them of the completion, or lack of completion, of one or more of these functions. Examples of the type of functions that controller 140 is configured to notify patient support apparatus server 84 of include the following: changing a state of a brake on patient support apparatus 20; changing a height of litter frame 28; changing an angle of head section 44; making other changes to the angle, position, and/or configuration of litter frame 28 and/or support deck 30; raising or lowering siderails 36; arming or disarming exit detection system 136; arming or disarming monitoring system 138; weighing a patient using scale system 144; zeroing, or making other changes to, scale system 144; adding or removing items from an equipment weight log maintained by patient support apparatus 20; deleting patient data stored in memory 134; entering new patient information into memory 134; locking or unlocking any one or more controls of patient support apparatus 20; power cycling patient support apparatus 20; plugging in or unplugging AC cord 150 into or out of an electrical outlet 108; plugging in or unplugging nurse call cable 66 into or out of cable port 148 and/or communication outlet 64; moving patient support apparatus 20 to a different location (as detected through the connection/disconnection of patient support apparatus 20 from multiple locator units 60); adjusting the inflation state of mattress 42; implementing a therapy and/or a patient turn using inflatable mattress 42; changing a setting of patient support apparatus 20; making other changes to the state of one or more components of patient support apparatus 20 and/or other UWB-equipped devices that are in UWB communication with patient support apparatus 20; and/or making other changes.

Each of these functions is carried out by manipulating one or more controls onboard patient support apparatus 20, and controller 140 is configured to assign a function identifier to each of these functions. Further, controller 140 is configured to forward these function identifiers to patient support apparatus server 84 after these functions are carried out. Patient support apparatus server 84 has a directory of function identifiers and the functions corresponding to these identifiers, which it uses to determine from the function identifier what functions have been performed on patient support apparatus 20. Patient support apparatus server 84 may then forward information about the functions that have been performed to one or more recipients, such as EMR server 92, one or more electronic devices 98, one or more computers on network 80 (e.g. computer 168; FIG. 8), and/or to other recipients. It may also, as noted, save data regarding these functions and/or display it on dashboard screen 200.

In some embodiments, controller 140 is configured to allow a user to customize what functions are transmitted to patient support apparatus server 84 for recordation, display, and/or forwarding. This customization may be carried out by an authorized user utilizing control panel 54c and/or by accessing patient support apparatus server 84. For example, controller 140 may be customized by an authorized user to not transmit any functions that are implemented on patient support apparatus 20 via the patient control panels 54c, but instead only transmit functions performed using caregiver controls panels 54a and/or 54c (and/or functions that don't require utilizing a control panel). As another example, controller 140 may be configured by an authorized user to not transmit changes to the positions of siderails 36, but to transmit changes to the brake, exit detection system 136, monitoring system 138, etc. Still other variations, of course, may be customized by authorized personnel.

Some functions that are completed by a user wearing a badge 142 may be desirably recorded in EMR server 92. For example, patient weight readings taken using scale system 144 may be desirably recorded in EMR server 92. Controller 140 is further customizable by the user as to which functions are to be recorded and automatically forwarded to EMR server 92. When controller 140 forwards these completed functions to EMR server 92, controller 140 also forwards caregiver identity information to EMR server 92 regarding the identity of the caregiver who implemented these functions. In some instances, the identity information includes the name and/or a unique identifier of the caregiver. Patient support apparatus server 84 determines the caregiver's name or unique identifier using the current location of patient support apparatus 20 and by receiving data from caregiver assignment server 104, which, as noted previously, may store the names of caregivers (or their unique IDs) and their corresponding assignments to specific rooms and/or bays.

Function data that is recorded on patient support apparatus 20 (e.g. in memory 134) and/or in patient support apparatus server 84 is subsequently retrievable by authorized personnel. Such data may be retrieved using a conventional computer, such as computer 168 (FIG. 8) that has access to network 80 and patient support apparatus server 84, by using an electronic device 98 that has access to network 80 and patient support apparatus server 84, and/or by one or more of the control panels 54 of patient support apparatus 20. By retrieving this information, authorized users can see not only the work performed by individual caregivers, but can also determine whether procedures, therapies, and/or treatments were completed in a proper and/or timely manner.

In some embodiments, after determining that the relative location of one or more of devices 100 and/or badges 142 are currently positioned within a corresponding space volume 152, controller 140 forwards a message to patient support apparatus server 84 indicating that these devices (whose IDs are included in the message) are currently located in the same room as patient support apparatus 20. Patient support apparatus server 84 may display this information on dashboard screen 200 (FIG. 8) and/or forward this information to one or more of electronic devices 98, thereby enabling authorized individuals to track the location of pieces of equipment (including badges 142) within the healthcare facility. Controller 140 may therefore, in addition to determining the location of UWB-equipped devices 100 (and badges 142) for purposes of communicating data to/from those devices, also determine the location of those devices 100 (or badges 142) for tracking purposes. Patient support apparatus 20 therefore can facilitate the tracking of a variety of equipment used within the healthcare facility for inventory management purposes and/or for performing timely service on the equipment.

In some embodiments, UWB transceivers 126, 126a, 132, 158, and 178 (FIGS. 5 & 7) may operate in the same manner as, and include any of the same functions as, the anchors and pseudo-anchors disclosed in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which has already been incorporated herein by reference. In some embodiments, locator units 60 may also be configured to determine the location of a device (e.g. device 100, badge 142) in any of the manners disclosed in commonly assigned U.S. patent application Ser. No. 63/132,514 filed Dec. 31, 2020, by inventors Alexander Bodurka et al. and entitled PATIENT SUPPORT APPARATUS AND MEDICAL DEVICE NETWORKS, and in commonly assigned U.S. patent application Ser. No. 63/154,677 filed Feb. 27, 2021, by inventors Celso Pereira et al. and entitled SYSTEM FOR DETERMINING PATIENT SUPPORT APPARATUS AND MEDICAL DEVICE LOCATION, the complete disclosures of both of which are incorporated herein by reference.

It will also be understood that, although FIG. 5 illustrates badge 142 as including a UWB transceiver 158, patient support apparatus 20 and patient support apparatus server 84 may be configured to implement any of the functions described herein using badges 142 that do not include a UWB transceiver 158. In such situations, patient support apparatus server 84 determines whether to associate a badge 142 with a particular patient support apparatus 20 based upon the location information that may be independently gathered by badge server 94 using conventional WiFi triangulation techniques (or other conventional location determination techniques). Although the location technology utilized by badge server 94 may not be as precise as the UWB location technology described herein, patient support apparatus server 84 may be configured to use this location information to determine whether to associate and disassociate non-UWB equipped badges 142 with patient support apparatuses 20.

Although the foregoing description has primarily indicated that controller 140 determines whether to associate or disassociate patient support apparatus 20 with one or more devices 100, badges 142, and/or locator units 60, it will be understood that the association and/or disassociation process may alternatively, partially, and/or additionally, be carried out by patient support apparatus server 84. In such embodiments, controller 140 forwards relative position information regarding these devices to patient support apparatus server 84 so that server 84 can determine whether to associate a particular device with a particular patient support apparatus 20. In such embodiments, server 84 sends a message back to patient support apparatus 20 informing it of what devices it should be associated with, and what devices it should not be associated with.

In some embodiments, server 84 is configured to associate or disassociate devices 100 with specific patients. Server 84 may implement this patient association and disassociation process by storing in its memory data correlating specific device identifiers 166 or 180—which are transmitted to patient support apparatus 20 by their corresponding devices, and then forwarded by patient support apparatus 20 to server 84 via network transceiver 96—with specific patient support apparatus identifiers 130. In order to associate specific devices with a specific patient, patient support apparatus server 84 uses the location identifier 122 that it receives from a specific patient support apparatus 20 to determine the room location of the specific patient support apparatus 20. From this room location, server 84 receives data from ADT server 102 that identifies a specific patient for a specific room (or bay within a room). Server 84 is then able to correlate a specific device 100 or badge 142 (associated with the specific patient support apparatus 20 in that room) with a specific patient identifier because it knows the room (or bay) of the patient support apparatus 20, the specific IDs of the devices associated with that particular patient support apparatus 20, and the patient identifier associated with that particular room (or bay). After associating a device with a particular patient, server 84 is able to automatically retrieve data from a specific patient's medical records stored within EMR server 92 and/or to automatically send data from a particular device to the specific patient's medical records stored in EMR server 92.

Although FIG. 5 illustrates a single device 100 and a single badge 142, it will be understood that this is merely for illustration purposes. Multiple devices 100 and/or badges 142 may be within communication range of UWB transceivers 132 of patient support apparatus 20 and controller 140 of patient support apparatus 20 is adapted to determine the locations of those devices relative to a corresponding space volume 152b, c, d, etc. and, if they are positioned inside of the corresponding space volume 152, to automatically associate them with patient support apparatus 20 and make their data available for display, forward their data to patient support apparatus server 84, and/or take any of the other actions described herein.

In some embodiments, controller 140 is adapted to automatically associate itself with any one or more of the devices disclosed in commonly assigned U.S. patent application 63/154,677 filed Feb. 27, 2021, by inventors Celso Pereira et al. and entitled SYSTEM FOR DETERMINING PATIENT SUPPORT APPARATUS AND MEDICAL DEVICE LOCATION, the complete disclosure of which is incorporated herein by reference. Patient support apparatus 20 is configurable by a user to also, or alternatively, automatically display any of the data from these other types of devices on any of the displays 52 and/or associated display devices 56 when these devices are associated with patient support apparatus 20.

It will be understood that the frequency at which controller 140 repetitively determines the relative location of devices 100, locator unit 60, and badges 142 may vary in different embodiments. In some embodiments, this occurs multiple times a second. In other embodiments, this occurs less frequently than once a second. In still other embodiments, the frequency at which controller 140 determines the relative position of a device to patient support apparatus 20 may vary according to the device, the current location of patient support apparatus 20, the movement state of patient support apparatus 20 (i.e. whether it is currently moving or stationary), the brake state of patient support apparatus 20 (i.e. whether the brake is on or off), and/or according to one or more other factors.

Controller 140 is configured to use the association status of a linked locator unit 60 (FIG. 4) to determine where to send data to for forwarding to the adjacent communication outlet 64, as well as to determine which linked locator unit 60 it will accept data from that originated from communication outlet 64. The associated linked locator unit 60 is the linked locator unit 60 that patient support apparatus 20 will send the patient's voice signals to for forwarding to nurse call system 70. It is also the linked locator unit 60 that controller 140 will send television commands to when a patient onboard patient support apparatus 20 activates one or more of the television controls 50l-50r. Similarly, it is the linked locator unit 60 that controller 140 will send light commands to when a patient onboard patient support apparatus 20 activates one or more or the reading or room light controls 50s or 50t. The linked locator unit 60 that patient support apparatus 20 associates itself with is also the linked locator unit 60 that patient support apparatus 20 will receive audio signals from and direct to its onboard speaker(s). Such audio signals may correspond to voice signals from a remotely positioned nurse that are forwarded to the corresponding communication outlet 64 by way of nurse call system 70, or such audio signals may correspond to television audio signals that are routed from television 72 to communication outlet 64 by way of the one or more conductors 68.

In any of the embodiments disclosed herein, server 84 may be configured to additionally execute a caregiver assistance software application of the type described in the following commonly assigned patent applications: U.S. patent application Ser. No. 62/826,97, filed Mar. 29, 2019 by inventors Thomas Durlach et al. and entitled PATIENT CARE SYSTEM; U.S. patent application Ser. No. 16/832,760 filed Mar. 27, 2020, by inventors Thomas Durlach et al. and entitled PATIENT CARE SYSTEM; and/or PCT patent application serial number PCT/US2020/039587 filed Jun. 25, 2020, by inventors Thomas Durlach et al. and entitled CAREGIVER ASSISTANCE SYSTEM, the complete disclosures of which are all incorporated herein by reference. That is, server 84 may be configured to share with one or more electronic devices 98 any of the information shared with the electronic devices disclosed in these aforementioned patent applications. Thus, for example, server 84 may be configured to not only share the location of patient support apparatuses 20 (and any devices that may be associated with them) with electronic devices 98, but it may also forward patient data to electronic devices 98 from one or more associated devices 100 (e.g. vital sign data, infusion pump data, etc.), patient support apparatus status data (e.g. current siderail position, bed exit status, brake status, motion lockout status, height status, scale data, etc.) badge 142 data, and/or caregiver rounding data (e.g. when the last rounding was performed for a particular patient, when the next rounds are due, etc.).

In some embodiments, locator units 60 may include additional information stored therein that is shared with patient support apparatus 20 when patient support apparatus 20 becomes associated with the locator unit 60. Such additional information may include location information identifying the relative position of the locator unit 60 with respect to one or more other locator units 60 that are positioned nearby. Additionally or alternatively, the locator units 60 may include information regarding the thickness and/or materials of the wall 62 to which it is attached, wherein such information provides an indication to the patient support apparatus 20 of the amount of attenuation that UWB signals will likely experience when traveling through that wall. Additionally or alternatively, the locator units 60 may include information identifying their general location within the healthcare facility (e.g. room 400, bay A of room 302, hallway X, maintenance area Y, radiology department, emergency department, etc.) and/or information identifying a more specific location of the locator units 60 within the healthcare facility (e.g. a set of X,Y,Z coordinates in a frame of reference that includes all, or a portion of, the healthcare facility; a height on the wall 62, a distance from one or more landmarks and/or architectural features within the healthcare facility, and/or other more specific information). In some embodiments, patient support apparatus 20 is adapted to utilize this information to determine its location within the healthcare facility and/or to determine whether it is positioned on the same side of the wall 62 as a particular locator unit 60. In some embodiments, patient support apparatus 20 and/or locator units 60 include any of the same structures, functions, and/or features of any of the patient support apparatuses and/or wall units disclosed in commonly assigned U.S. patent application Ser. No. 63/245,245 filed Sep. 17, 2021, by inventors Kirby Neihouser et al. and entitled SYSTEM FOR LOCATING PATIENT SUPPORT APPARATUSES, the complete disclosure of which has already been incorporated herein by reference.

In some embodiments, controller 140 is also configured to automatically record information that goes into a log of events. The log of events lists the time, the people involved, any equipment that may be involved, and information about the events recorded therein. For example, in some embodiments, controller 140 is adapted to automatically record any time a component of the patient support apparatus 20 has a state of one or more of its components changed, as well as the time of the change, any people that were within a space volume 152 of patient support apparatus 20 when the change occurred, and/or any equipment (e.g. devices 98 and/or 100) that was within a space volume 152 of patient support apparatus 20 when the change occurred. By recording all of this information, controller 140 and/or server 84 is able to generate a log that associates events with time, people, equipment, and the actual event. Controller 140 and/or server 84 may create and/or update this log.

As noted, the list of events that are included in the log may include any change in the state of one or more components of patient support apparatus 20 and/or any changes in the state of any components of any UWB-equipped equipment (e.g. devices 100) that are positioned within range patient support apparatus 20. Such changes in the state of a patient support apparatus 20 may include any of the functions previously mentioned above that controller 140 is configured to notify patient support apparatus server 84 of, such as, but not limited to, changing a state of a brake on patient support apparatus 20; changing a height of litter frame 28; changing an angle of head section 44; making other changes to the angle, position, and/or configuration of litter frame 28 and/or support deck 30; raising or lowering siderails 36; arming or disarming exit detection system 136; arming or disarming monitoring system 138; weighing a patient using scale system 144; zeroing, or making other changes to, scale system 144; adding or removing items from an equipment weight log maintained by patient support apparatus 20; deleting patient data stored in memory 134; entering new patient information into memory 134; locking or unlocking any one or more controls of patient support apparatus 20; power cycling patient support apparatus 20; plugging in or unplugging AC cord 150 into or out of an electrical outlet 108; plugging in or unplugging nurse call cable 66 into or out of cable port 148 and/or communication outlet 64; moving patient support apparatus 20 to a different location (as detected through the connection/disconnection of patient support apparatus 20 from multiple locator units 60); adjusting the inflation state of mattress 42; implementing a therapy and/or a patient turn using inflatable mattress 42; changing a setting of patient support apparatus 20; activating, using, or deactivating propulsion system 212; activating or deactivating any of the actuators 216 aboard patient support apparatus 20; or making other changes.

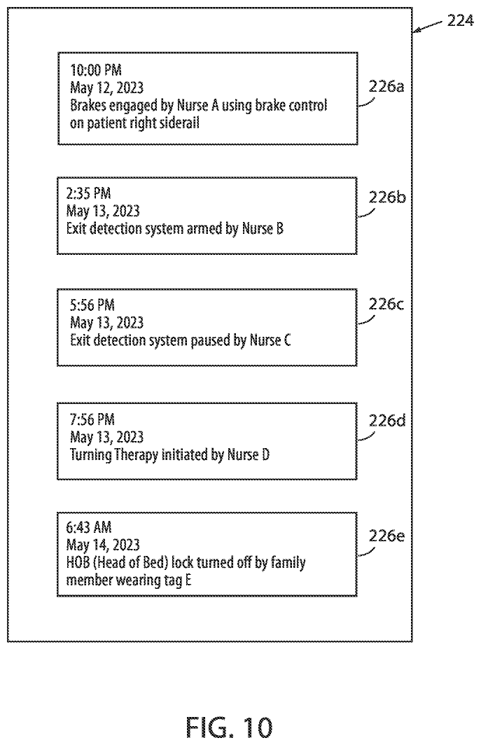

FIG. 10 illustrates one example of a log 224 that may be maintained by patient support apparatus server 84 and/or one or more individual patient support apparatuses 20. Log 224 includes one or more events 226a-e that are automatically detected by controller 140 of patient support apparatus 20. As these events are detected, controller 140 uses the UWB transceivers 132 of patient support apparatus 20 to determine who is present when the events occur and what equipment is present when these events occur. In other words, controller 140 is configured to take a UWB snapshot of all of the UWB devices that are currently within communication range of patient support apparatus 20 at the time the event is detected. The UWB devices that may be part of this snapshot include badges 142, locators 60, equipment tags attached to equipment (such as devices 100 that have separate UWB tags), devices 100 with built-in UWB communication circuitry (as opposed to devices 100 with a separate UWB tag, electronic devices 98, and/or other devices. In some embodiments, controller 140 only takes a snapshot of UWB devices that patient support apparatus 20 is currently associated with at the moment the event occurs, while in other embodiments, controller 140 takes a snapshot of all UWB devices that transceiver 132 are in communication range with at the moment the event occurs.

In the example shown in FIG. 10, log 224 includes a first event 226a that corresponds to the brake 214 of patient support apparatus 20 being activated. As can be seen in FIG. 10, controller 140 is configured to time stamp (and date) the moment that it detects that its brake 214 has been activated. As can also be seen in FIG. 10, controller 140 has detected that the badge 142 of Nurse A was either within communication range of transceivers 132 at the moment the brake 214 was activated, or that the badge 142 of Nurse A was within a volume of space 152 defined in the vicinity of patient support apparatus 20 at the moment brake 214 was activated. In some embodiments, controller 140 adds this information to log 224 and stores the log 224 within memory 134 onboard patient support apparatus 20. In other embodiments, controller 140 merely transmits this information to server 84 and server 84 stores this information in a log 224 maintained by server 84. In still other embodiments, both patient support apparatus 20 and server 84 may maintain a log of events detected by controller 140.

Controller 140 concludes that Nurse A activated the brake 214 on patient support apparatus 20 as part of event 226a (FIG. 10) because controller 140 detects the badge 142 of Nurse A is within a threshold distance (i.e. a particular volume of space 152) of the brake control at the moment the brake control is activated. In this particular example, the brake was activated using a control on the caregiver control panel 54b located on the right siderail 36 (from the patient's perspective, when lying on their back). Accordingly, when the brake control on the right siderail 36 is activated, controller 140 looks for any badges 142 that are positioned within a threshold distance (e.g. within human reach) of the right siderail 36 of patient support apparatus 20. If any such badges are detected (using UWB transceivers 132), controller 140 records the location of the badge(s) 142, consults server 84 to determine the identity of the person(s) associated with those badge(s) 142, and then stores that information in log 224 (and/or server 84 may store that information in log 224).

The other events illustrated in FIG. 10 include the detection of the arming of exit detection system 136 by Nurse B (event 226b); the pausing of exit detection system 136 by Nurse C (event 226c); the initiating of a turning therapy using an inflatable mattress 42 by Nurse D (event 226d); and the Head of Bed (HOB) lock being turned off by a family member wearing tag E (event 226e). This last event 226e refers to the unlocking of a limit on the pivoting range of head section 44, thus enabling the head section 44 to pivot throughout its entire range of motion. For event 226e, at least one of the family members has been given a UWB tag during their visit to the hospital, and controller 140 uses transceivers 132 to detect the presence of the family member's tag when it is within communication range.

In some instances, if controller 140 is not able to determine which individual carried out a particular action of an event 226, controller 140 may be configured to record the event 226 in the log with a notation that the person responsible for the event could not be determined, or that no badges 142 or visitor tags were detected within a threshold distance of patient support apparatus 20 at the time of the event 226.

In some embodiments, controller 140 is configured to take a snapshot of some or all of the UWB devices (locators 60, devices 98, 100, badges 142, UWB tags, and/or other UWB devices) whenever a state of a component of patient support apparatus 20 changes. In other embodiments, controller 140 may be configured to take the same or a similar type of snapshot at periodic time intervals. In still other embodiments, controller 140 may be configured to take both time-based snapshots and event-based snapshots and add them to log 224 (or forward them to server 84 for adding to the log 224). When controller 140 is configured to take time-based snapshots, controller 140 may vary the intervals between the snapshots based on one or more factors, including, but not limited to, the absence/presence of any badges 142, the absence/presence of any particular equipment (e.g. devices 98 and/or 100), the location of patient support apparatus 20 within the healthcare facility, the time of day, the date, and/or other factors.

Although not shown in FIG. 10, controller 140 may be further configured to add to log 224 any UWB equipment, such as any devices 100, that are detected at the moment an event is detected (or at the moment a time-based snapshot is taken). Thus, for example, event 226a in FIG. 10 might also include information about a DVT pump that is detected on patient support apparatus 20 at that moment, or an infusion pump that is positioned on or near patient support apparatus 20 at that moment, or any other equipment (e.g. devices 98, 100) that are within a threshold distance (or communication range) of patient support apparatus 20. Controller 140 and/or server 84 determine the identity of any equipment that is present at the moment the snapshot is taken by using the unique identifier (1980, 222, and/or 166) received from the equipment to consult a table (stored in, or accessible to, server 84) that correlates the unique identifiers to particular pieces, or types of pieces, or equipment.

In some embodiments, controller 140 is configured to not only record the presence of badges, equipment, tags, and/or other UWB-equipped devices, but also to record the three-dimensional positions of such badges, equipment, tags, and/or other UWB-equipped devices when making an entry into log 224. In such embodiments, log 224 will contain the three-dimensional position of any badges 142, devices 98, 100, and/or locator units 60 at the moment a snapshot is taken.

In some embodiments, in addition to, or in lieu of, adding events 226 to log 224, controller 140 may be configured to issue one or more notifications when no caregiver is detected within the room of patient support apparatus 20 (e.g. no badge 142 is present), or otherwise within the vicinity of patient support apparatus 20, if one or more components of patient support apparatus 20 are potentially in undesired states. For example, if brake 214 is not active, controller 140 may send a notification to server 84, which in turn may forward the notification to one or more badges 142 and/or electronic devices 98. As another example, if exit detection system 136 has not been armed, controller 140 may send another notification to server 84. Still other notifications may be issued for still other potentially undesirable conditions of patient support apparatus 20.

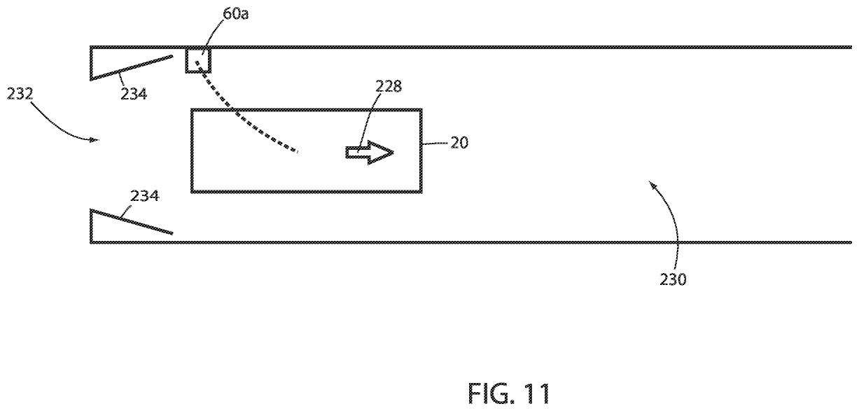

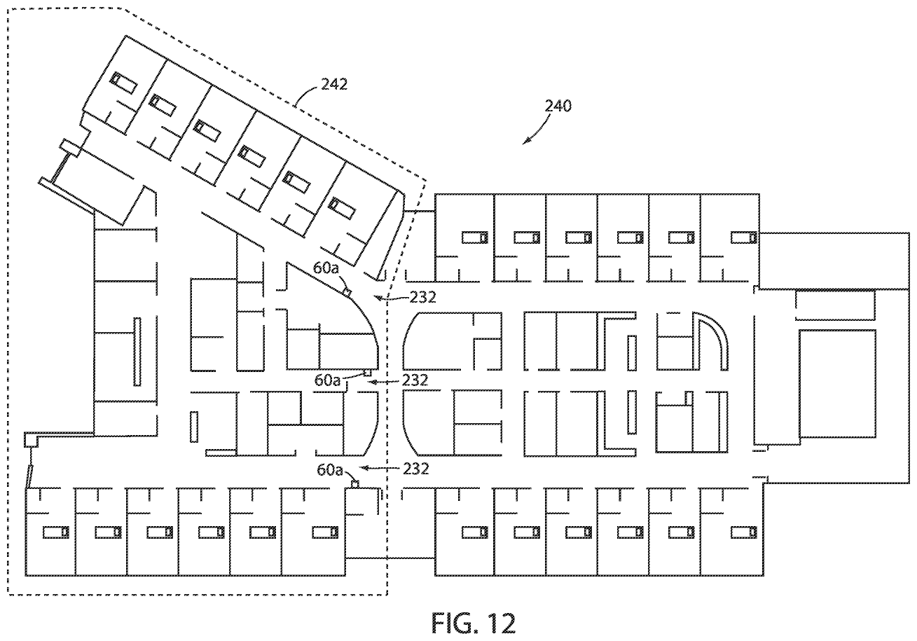

In some embodiments, controller 140 and/or certain unlinked locator units 60a are configured to allow the direction of movement of patient support apparatus 20 to be determined as patient support apparatus 20 moves past such an unlinked locator unit 60a. In such embodiments, controller 140 and/or an adjacent unlinked locator unit 60a determines the direction of movement of patient support apparatus 20 and forwards this information to patient support apparatus server 84. Server 84 keeps track of this information and shares it with other computers (e.g. computer 168, electronic devices 98, etc.) so that personnel of the healthcare facility can determine generalized areas into which the patient support apparatus 20 has entered or exited. Examples of such embodiments are described in greater detail below with respect to FIGS. 11 and 12.

In some instances, personnel at a healthcare facility may be interested in knowing a generalized location (e.g. non-room specific location) of patient support apparatus 20, or information about what generalized area of the healthcare facility the patient support apparatus 20 had last entered or exited. Rather than placing locator units 60 (linked and/or unlinked) at all locations within the healthcare facility so that the patient support apparatus 20 is never outside of communication range with at least one locator unit 60, the personnel of the healthcare facility may be interested in a less costly method of tracking the whereabouts of the patient support apparatuses 20 within the healthcare facility. In such situations, controller 140 and/or server 84 may be configured to determine the direction of movement of patient support apparatus 20 as it passes by one or more unlinked locator units 60a that are strategically placed at entrances to areas of interest in the healthcare facility, thereby enabling patient support apparatus 20 and/or server 84 to determine if the patient support apparatus 20 has entered, or exited, these areas of interest.

An example of this direction determination is shown in FIG. 11. In FIG. 11, a patient support apparatus 20 is shown moving in a direction 228 down a hallway 230. The hallway has an entrance (and exit) 232 with two doors 234 positioned thereat. Adjacent one of the doors 234 is an unlinked locator unit 60a mounted to one of the walls of the hallway 230. As patient support apparatus 20 moves past unlinked locator unit 60a, transceivers 132 of patient support apparatus 20 communicate with the UWB transceiver 126a positioned onboard locator unit 60a and determine their relative positions. Utilizing additional information that is stored onboard patient support apparatus 20, unlinked locator unit 60a, and/or server 84, controller 140 (or unlinked locator unit 60a or server 84) is able to determine in which direction patient support apparatus 20 is moving. That is, controller 140 (or unlinked locator unit 60a or server 84) determines whether patient support apparatus 20 is moving further down hallway 230 in the direction 228, or whether patient support apparatus 20 is moving in the opposite direction and exiting hallway 230 (by exiting through doors 234).

Another unlinked locator unit 60a (not shown) may be positioned at the opposite end of hallway 230 so that when patient support apparatus 20 passes by that other unlinked locator unit 60a, controller 140 will know that patient support apparatus 20 has exited out the opposite end of hallway 230. Alternatively, or additionally, additional unlinked locator units 60a may be placed in intermediate positions along hallway 230 if greater location granularity is desired as to the specific location of patient support apparatus 20 within hallway 230.

In some instances, a healthcare facility may position one or more unlinked locator units 60a at each of the entrances (which may also be exits) into one or more areas of the healthcare facility. One example of this is shown in FIG. 12. FIG. 12 illustrates an illustrative healthcare facility floorplan 240. An area 242 is shown in dashed lines overlaid on top of floorplan 240. If the personnel at the healthcare facility are interested in knowing whether or not a patient support apparatus 20 is currently within area 242, the healthcare facility has unlinked locator units 60a installed at each of the entrances 232. As a patient support apparatus 20 moves by one of these locator unit 60a, its direction of movement is determined so that controller 140 and/or server 84 are able to determine whether the patient support apparatus 20 is moving into area 242, or exiting from area 242. Regardless of which direction patient support apparatus 20 is moving in, server 84 (or controller 140) record the time of the movement past the locator unit 60, as well as the direction of movement (into, or out of, area 242). This information can then be shared with other computer devices (e.g. computer 168, electronic devices 98, badges 142, etc.) when a user inquires as to the last known location of patient support apparatus 20. Because there are locator units 60 positioned at each of the entrances 232 of area 242, it is not possible for patient support apparatus 20 to subsequently enter or exit area 242 without it being detected. When such a subsequent entry or exit is detected, the time is recorded, as well as whether the subsequent detection is an entry or an exit. In this manner, a log may be maintained of the movement of patient support apparatus 20 throughout the facility, as well as its last known location with respect to area 242 (and/or with respect to other areas).

In some instances, an area 242 may include one or more sub-areas that are defined by entrances 232 that each include one or more unlinked locator units 60a positioned nearby. In some instances, a particular area 242 or sub-area may also include one or more linked locator units 60 that are positioned within one or more rooms of the area 242 or sub-area. When patient support apparatus 20 associates itself with one of these linked locator units 60, the room location of patient support apparatus 20 is able to be determined, thereby allowing personnel to not only know what area 242 the patient support apparatus 20 is positioned in, but also the specific room within area 242. In other instances, one or more areas 242 or sub-areas may not include any linked locator units 60 within that area or sub-area, in which case only the generalized (i.e. non-room specific) location of patient support apparatus 20 within that area or subarea can be determined. Area 242 and/or their sub-areas may refer to any area of the healthcare facility that is of interest to the personnel of that healthcare facility, such as, but not limited to, hallways, departments, wings, buildings, elevators, floors, units, etc.

Although ultra-wideband signals generally do not pass through ceilings without substantial attenuation, it may be desirable to place an unlinked locator unit 60a adjacent to each elevator on each floor of a healthcare facility. In this manner, the movement of a patient support apparatus 20 into and out of the elevator can be determined. Further, because server 84 (and/or patient support apparatus 20) maintains a list of which floor each locator unit 60a (and 60) is positioned on, server 84 (and/or patent support apparatus 20) is able to keep track of which floor patient support apparatus 20 is currently located on. In some instances, where ultra-wideband signals may pass through floors, the height of unlinked locator units 60a on the wall (or other architectural structure) to which they are mounted may be recorded, as well the dimensions of the entrance 232 adjacent to the unlinked locator unit 60a. Knowing this height and the entrance dimensions, controller 140 (and/or server 84) can rule out communications between an unlinked locator unit 60a and a patient support apparatus 20 that is positioned on a floor different from the one on which locator unit 60a is located. This can be particularly useful in situations where the distance between an unlinked locator unit 60a and a patient support apparatus 20 positioned directly underneath or, or above it, on a different floor, is larger than the dimensions of the entrance 232. Further, in some embodiments, in order to reduce the likelihood of locator units 60 communicating with patient support apparatuses 20 on different floors, shielding may be applied to the top and/or bottom of the locator unit 60 so as to reduce the communication range of the locator unit 60 in the vertical direction. One example of such shielding being applied to ultra-wideband locator units is disclosed in commonly assigned PCT patent application serial number PCT/US2022/043585 filed Sep. 15, 2022, by Stryker Corporation and entitled SYSTEM FOR LOCATING PATIENT SUPPORT APPARATUSES, the complete disclosure of which is incorporated herein by reference.

The manner in which controller 140, unlinked locator unit 60a, and/or server 84 determines the direction of movement of patient support apparatus 20 as it moves past locator unit 60a may vary. In some embodiments, patient support apparatus 20 includes a compass onboard that tells controller 140 in which geographical direction it is moving (e.g. north, east, south, etc.) and data is stored indicating which geographical direction of movement corresponds to an exit and an entry from a particular area 242. For example, in the situation illustrated in FIG. 11, data may be stored onboard patient support apparatus 20, somewhere accessible to server 84, and/or onboard locator unit 60a that indicates that when a patient support apparatus 20 moves past the locator unit 60a traveling east (e.g. direction 228), the patient support apparatus is proceeding down hallway 230, and data indicating that when patient support apparatus 20 moves past the locator unit 60a traveling west, it will be exiting hallway 230 (through doors 234). From this information, controller 140, server 84, and/or locator unit 60a can determine whether the patient support apparatus 20 is exiting or entering a particular area 242 as it moves past locator unit 60a.

In another embodiment, controller 140 may determine its direction of movement relative to an area 242 of a healthcare facility by placing two or more locator units 60a in sequential position along the entrance 232 to a particular area, and storing this sequential position in a memory accessible to controller 140. When placed sequentially, patient support apparatus 20 will be closer to one of the locator units 60a as it approaches the entrance 232. Based on which one of the two locator units 60a that it is closer to as it approaches the entrance (or as it moves away from the entrance), the direction of movement of patient support apparatus 20 relative to the entrance 232 (e.g. whether into the entrance or out of the entrance) can be determined. Still other manners of determining the direction of movement may be implemented, including manners disclosed in the aforementioned PCT application serial number PCT/US2022/043585 filed Sep. 14, 2022, by Stryker Corporation and entitled SYSTEM FOR LOCATING PATIENT SUPPORT APPARATUSES, the complete disclosure of which has been incorporated herein by reference.

In some embodiments, patient support apparatus 20 is configured to determine if any UWB-equipped devices are in a motion or movement path of a component of patient support apparatus 20 prior to activating the actuator 216 that is responsible for moving that component. This helps prevent components of patient support apparatus 20 from being moved into people, equipment, and/or other obstacles, thereby preventing injury to people and damage to patient support apparatus 20 itself and/or other objects. More specifically, in such embodiments, controller 140 is configured to use UWB transceivers 132 to determine the three-dimensional position of any badges 142, electronic devices 98, or other devices 100 prior to moving any components of patient support apparatus 20. If any of these devices (142, 98, 100) are detected within communication range of patient support apparatus 20, controller 140 determines whether their three-dimensional position lies within the path of movement of one or more components of patient support apparatus 20. If any of these devices lie within one or more motion paths of any moveable components of patient support apparatus 20, controller 140 prevents movement of that component, either completely, or in whichever direction will move the component closer to the device.

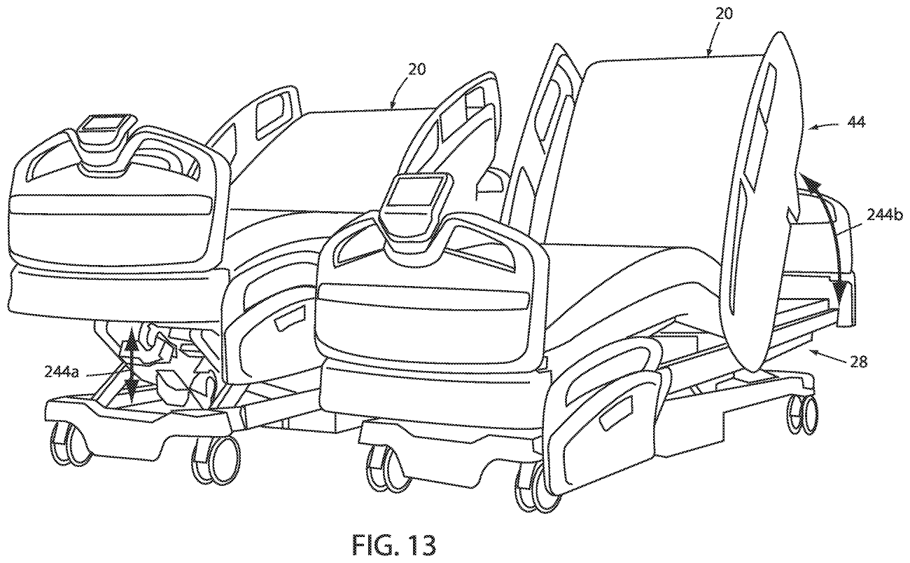

FIG. 13 illustrates two examples of the movement path detection. In FIG. 13, a first motion path 244a is shown that represents the movement path of litter frame 28 as its height is raised or lowered. FIG. 13 also illustrates a second movement path 244b of head section 44 as it pivots about a generally horizontal axis defined near the foot end of head section 44. Memory 134 of patient support apparatus 20 includes data indicating the three-dimensional volume of space that litter frame 28 occupies as it is raised or lowered along movement path 244a. Memory 134 also includes data indicating the three-dimensional volume of space that head section 44 occupies as it is pivoted upward or downward. Controller 140 compares these three dimensional volumes of space to the three-dimensional positions of any badges 142 or other UWB devices 98, 100 that may present an obstacle to the movement of these components and prevents movement of the component if a badge or other UWB device 98, 100 is within a threshold distance of these three-dimensional volumes of space.

The threshold distance utilized may vary depending upon the direction of movement, the particular movable component, the particular badge 142 and/or device 98, 100, or based on other factors. In general, the threshold distance should account for tolerances in the ability of controller 140 to accurately determine the three-dimensional position of a badge 142 or device 98, 100 using UWB transceivers 132. Additionally, the threshold distance may take into account the fact that a person wearing a badge 142 has limbs and body parts that extend outwardly from the UWB transceiver 158 within badge 142, and therefore, although the badge 142 may be outside of a movement path 244, the person's arms, legs, or other portion of their body might still be within a motion path. For example, if a UWB-equipped cell phone 100 is detected at a position lower than the current height of litter frame 28, but outside of its motion path 244, controller 140 may be configured to still prevent downward movement of litter frame 28 because the cell phone may be in the person's pocket and his/her arm, leg, or torso may be positioned within the movement path of litter frame 28.

In some embodiments, controller 140 is configured to not prevent movement of any component of patient support apparatus 20 when such component is moved in a direction that is intended to lift the patient, or a portion of the patient, regardless of the presence of a badge 142 or device 98, 100 within the component's movement path 244. For example, if the patient's UWB-equipped cell phone is detected to be lying on mattress 42, controller 140 is configured to allow lifts 26 to raise litter frame 28, despite the fact that the patient's UWB-equipped cell phone is positioned within the upward motion path of litter frame 28. This is because such lifting movement is part of the normal movement of patient support apparatus 20 that is intended to reposition the patient and/or objects positioned on the litter frame. In contrast, controller 140 may be configured to prevent movement of any component of patient support apparatus 20 in a downward direction if a UWB-equipped object is detected within the component's motion path 244. Thus, for example, if a caregiver badge 142 is detected underneath litter frame 28 (or within a threshold distance thereof), controller 140 may be configured to prevent lowering of litter frame 28 until the badge 142 is moved further away from the motion path 244 of litter frame 28.

In some embodiments of patient support apparatus 20, controller 140 may be configured to take other actions when a UWB-equipped device is detected within a threshold distance of a motion path of a moveable component, other than, or in addition to, preventing movement of the component further toward the UWB-equipped device. For example, in some embodiments, controller 140 may issue a warning prior to allowing such movement. The warning may require acknowledgement on one of control panels 54 that such motion is indeed desired, despite the risk of injury or damage. Alternatively, or additionally, controller 140 may allow such movement, but do so at a reduced speed. In those embodiments of patient support apparatus 20 that include an obstruction detection system, controller 140 may increase the sensitivity level of the obstruction detection system when a UWB-equipped device is detected within the threshold distance of the movement path of a component, thereby reducing the risk of serious injury or damage if a collision occurs. Still further, controller 140 may be configured in some embodiments to change its reaction to a UWB-equipped device being detected within a threshold distance of the motion path 244 based upon how close the device is to the motion path. Still other variations are possible.

Controller 140 determines whether a UWB-equipped device (e.g. badge 142 or device 98, 100) is within a component's motion path by using data stored in memory 134 that relates the three-dimensional position of each of the UWB transceivers 132 to the three-dimensional volume encompassed by each movable component of patient support apparatus 20 as the component moves through its respective motion path. In other words, memory 134 includes data relating the positions of transceivers 132 to the different positions of the movable components onboard patient support apparatus 20.

It will be understood by those skilled in the art that the use of the term “transceiver” throughout this specification is not intended to be limited to devices in which a transmitter and receiver are necessarily within the same housing, or share some circuitry. Instead, the term “transceiver” is used broadly herein to refer to both structures in which circuitry is shared between the transmitter and receiver, and transmitter-receivers in which the transmitter and receiver do not share circuitry and/or a common housing. Thus, the term “transceiver” refers to any device having a transmitter component and a receiver component, regardless of whether the two components are a common entity, separate entities, or have some overlap in their structures.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

1. A patient support apparatus comprising:
a support surface adapted to support a patient;
a plurality of ultra-wideband transceivers;
a sensor adapted to detect an undesired state of the patient support apparatus;
a network transceiver adapted to communicate with a healthcare facility computer network; and
a controller adapted to use radio frequency (RF) communication between the plurality of ultra-wideband transceivers and a badge to determine a position of the badge relative to the patient support apparatus; the controller further adapted to receive a badge identifier from the badge; to determine if the badge is positioned inside or outside of a volume of space; to issue an alert if the badge is positioned outside of the volume of space while the undesired state exists; and to stop the alert if the badge is positioned inside the volume of space while the undesired state exists.

2. The patient support apparatus of claim 1 wherein the controller is configured to stop the alert for as long as the badge remains inside the volume of space and the undesired state continues to exist.

3. The patient support apparatus of claim 1 wherein the sensor is adapted to detect an exit of the patient from the patient support apparatus, and the undesired state is the patient exiting from the patient support apparatus.

4. The patient support apparatus of claim 1 further comprising a plurality of siderails movable between raised and lowered positions, and wherein the sensor is adapted to detect a position of at least one of the siderails, and the undesired state is the at least one of the siderails being moved to its lowered position.

5. The patient support apparatus of claim 1 further comprising a sound emitting device, wherein the controller is further adapted to cause the sound emitting device to emit an alert sound when the controller issues the alert, and to cause the sound emitting device to not emit the alert sound when the controller stops the alert.

6. The patient support apparatus of claim 1 further wherein the controller is adapted to issue the alert by sending an alert message to a server hosted on the healthcare facility computer network, and to stop the alert by sending an alert cancellation message to the server hosted on the healthcare facility computer network.

7. The patient support apparatus of claim 1 further comprising a headwall interface adapted to communicate with a communication outlet integrated into a wall of the healthcare facility, wherein the communication outlet includes a plurality of pins and the controller is adapted to issue the alert by setting an electrical state of at least one pin to a first state, and to stop the alert by setting the electrical state of the at least one pin to a second state.

8-23. (canceled)

24. A patient support apparatus comprising:
a support surface adapted to support a patient;
a plurality of ultra-wideband transceivers;
a sensor adapted to detect an undesired state of the patient support apparatus;
a network transceiver adapted to communicate with a healthcare facility computer network; and
a controller adapted to use radio frequency (RF) communication between the plurality of ultra-wideband transceivers and a badge to determine a position of the badge relative to the patient support apparatus, to use radio frequency (RF) communication between the plurality of ultra-wideband transceivers and a tag coupled to the patient to determine a position of the tag relative to the patient support apparatus, and to automatically determine a distance between the tag and the badge.

25. The patient support apparatus of claim 24 wherein the controller is further adapted to send a message to the healthcare facility computer network if the distance exceeds a threshold.

26. The patient support apparatus of claim 25 wherein the controller is further adapted to vary the threshold based on a condition of the patient.

27. The patient support apparatus of claim 26 wherein the condition is a fall risk assessment that exceeds a predetermined value.

28. The patient support apparatus of claim 25 further comprising a sensor adapted to detect an undesired state of the patient support apparatus; wherein the controller is further adapted to determine if the badge is positioned inside or outside of a volume of space; to issue an alert if the badge is positioned outside of the volume of space while the undesired state exists and the distance is less than the threshold; and to stop the alert if the badge is positioned inside the volume of space while the undesired state exists and the distance is less than the threshold.

29-34. (canceled)

35. The patient support apparatus of claim 28 wherein the controller is further adapted to issue the alert by sending an alert message to the badge, and to stop the alert by sending a cancellation message to the badge.

36-134. (canceled)

135. A patient support apparatus comprising:
a support surface adapted to support a patient;
a plurality of ultra-wideband transceivers; and
a controller adapted to use radio frequency (RF) communication between the plurality of ultra-wideband transceivers and a badge to determine a position of the badge relative to the patient support apparatus, the controller further adapted to receive a badge identifier from the badge, to determine if a state of a component of the patient support apparatus changes, and to store in memory a log containing the badge identifier of any badge that was within a threshold distance of the patient support apparatus when the state of the component changed.

136. The patient support apparatus of claim 135 wherein the controller is further adapted to use RF communication between the plurality of ultra-wideband transceivers and an equipment tag to determine a position of the equipment tag relative to the patient support apparatus, to receive an equipment tag identifier from the equipment tag, and to store in the log the equipment tag identifier of any equipment tag that was within a second threshold distance of the patient support apparatus when the state of the component changed.

137. The patient support apparatus of claim 136 wherein the second threshold distance is different from the threshold distance.

138. The patient support apparatus of claim 136 wherein the second threshold distance is the same as the threshold distance.

139. The patient support apparatus of claim 135 wherein the controller is adapted to determine the position of the badge relative to the patient support apparatus in three dimensions, and to store in the log the three-dimensional position of the badge if the badge was within the threshold distance of the patient support apparatus when the state of the component changed.

140. (canceled)

141. The patient support apparatus of claim 135 wherein the controller is further adapted to use RF communication between the plurality of ultra-wideband transceivers and a smart phone equipped with a UWB transceiver to determine a position of the smart phone relative to the patient support apparatus, to receive a smart phone identifier from the smart phone, and to store in the log the smart phone identifier of any smart phone that was within a third threshold distance of the patient support apparatus when the state of the component changed.

142. The patient support apparatus of claim 141 wherein the controller is adapted to determine the position of the smart phone relative to the patient support apparatus in three dimensions, and to store in the log the three-dimensional position of the smart phone if the smart phone was within the third threshold distance of the patient support apparatus when the state of the component changed.

143-251. (canceled)
